(19) United States
(12) Patent Application Publication
(10) Pub. No.: US 2026/0335409 A1
(43) Pub. Date: Oct. 29, 2026

(54) COMPOSITIONS AND METHODS FOR THE TREATMENT OF CORNEAL DISEASES AND DISORDERS

(71) Applicant: INDIANA UNIVERSITY RESEARCH AND TECHNOLOGY CORPORATION, Bloomington, IN (US)

(72) Inventors: Konstantin FEINBERG, Indianapolis, IN (US); Gregory H. BORSCHEL, Indianapolis, IN (US)

(21) Appl. No.: 19/486,288

(22) PCT Filed: May 24, 2024
(86) PCT No.: PCT/US2024/031006

§ 371 Date: Nov. 20, 2025

Related U.S. Application Data
(60) Provisional application No. 63/504,388, filed on May 25, 2023.

(51) Int. Cl. A61K 38/18; A61K 9/00; A61P 27/02

(52) U.S. Cl. CPC A61K 38/185; A61K 9/0048; A61K 38/18; A61K 38/1841; A61K 38/1858; A61P 27/02

(57) ABSTRACT

Disclosed are pharmaceutical compositions comprising trophic factors and methods for treating corneal diseases or disorders, e.g., neurotrophic keratopathy, diabetic keratopathy, using the same. The pharmaceutical compositions may comprise, e.g., ciliary neurotrophic factor (CNTF) and at least one additional trophic factor selected from the group consisting of pleiotrophin (PTN), transforming growth factor beta 1 (TGFb1), and platelet-derived growth factor alpha (PDGFa) or comprise PTN or CNTF and PTN.

### CROSS-REFERENCE TO RELATED APPLICATIONS

The present application claims priority to U.S. Provisional Patent Application No. 63/504,388, that was filed May 25, 2023, the entire contents of which are incorporated by reference herein.

### SEQUENCE LISTING

A Sequence Listing accompanies this application and is submitted as an xml file of the sequence listing named “144578_00403.xml” which is 5,340 bytes in size and was created on Apr. 26, 2024. The sequence listing is electronically submitted via Patent Center and is incorporated herein by reference in its entirety.

### BACKGROUND

Corneal epithelial maintenance and wound healing depend on the activity of limbal stem cells (LSCs) residing in the basal epithelium of the limbus. Impaired corneal sensory innervation interferes with the activity of LSC-dependent corneal epithelial renewal, leading to corneal ulceration, opacification, and inevitable blindness. This condition, neurotrophic keratopathy (NK), affects 5/10,000 people worldwide. Topical recombinant human nerve growth factor (rhNGF) is the only FDA-approved drug for treating NK. However, NGF is not effective in 40% of patients. In addition, extremely high dosage of NGF (8 weeks of treatment of 20 μg/ml/6 times daily eyedrops) make this approach highly expensive ($100,000 per course) and cumbersome for the patient. Accordingly, there is a need in the art for novel treatments for corneal diseases and disorders, e.g., neurotrophic keratopathy.

### SUMMARY

In an aspect of the current disclosure, pharmaceutical compositions are provided. In some embodiments, the pharmaceutical compositions comprise ciliary neurotrophic factor (CNTF) and at least one additional trophic factor selected from the group consisting of pleiotrophin (PTN), transforming growth factor beta 1 (TGFb1), and platelet-derived growth factor alpha (PDGFa). In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound.

In an aspect of the current disclosure, methods of treating neurotrophic keratopathy in a subject in need thereof are provided. In some embodiments, the methods comprise administering a therapeutically effective amount of a pharmaceutical composition comprising ciliary neurotrophic factor (CNTF) and at least one additional trophic factor selected from the group consisting of: pleiotrophin (PTN), transforming growth factor beta 1 (TGFb1), and platelet-derived growth factor alpha (PDGFa) to the subject to treat the neurotrophic keratopathy. In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound. In some embodiments, administration comprises topical ophthalmic administration. In some embodiments, the therapeutically effective amount of the pharmaceutical composition comprises PTN at a concentration of about 12.5 ng/ml in the pharmaceutical composition. In some embodiments, the therapeutically effective amount of the pharmaceutical composition comprises CNTF at a concentration of about 25 ng/ml in the pharmaceutical composition. In some embodiments, the therapeutically effective amount of the pharmaceutical composition comprises TGFb1 at a concentration of about 2.5 ng/ml in the pharmaceutical composition. In some embodiments, the therapeutically effective amount of the pharmaceutical composition comprises PDGFa at a concentration of about 25 ng/ml in the pharmaceutical composition. In some embodiments, the method improves corneal transparency in the subject. In some embodiments, the subject has about 50% or less corneal innervation, about 40% or less corneal innervation, about 30% or less corneal innervation, about 20% or less corneal innervation, about 10% or less corneal innervation, or wherein the subject has no corneal innervation. In some embodiments, the subject suffers from corneal anesthesia. In some embodiments, the corneal anesthesia is congenital corneal anesthesia. In some embodiments, the subject is suffering from neurotrophic keratopathy. In some embodiments, the method results in complete recovery of the corneal epithelium. In some embodiments, the subject is suffering from diabetes.

In another aspect of the current disclosure, methods of improving corneal healing in a subject in need thereof are provided. In some embodiments, the methods comprise administering a therapeutically effective amount of a pharmaceutical composition comprising ciliary neurotrophic factor (CNTF) and at least one additional trophic factor selected from the group consisting of: pleiotrophin (PTN), transforming growth factor beta 1 (TGFb1), and platelet-derived growth factor alpha (PDGFa) to the subject to improve corneal healing in the subject. In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound. In some embodiments, administration comprises topical ophthalmic administration. In some embodiments, the therapeutically effective amount of the pharmaceutical composition comprises PTN at a concentration of about 12.5 ng/ml in the pharmaceutical composition. In some embodiments, the therapeutically effective amount of the pharmaceutical composition comprises CNTF at a concentration of about 25 ng/ml in the pharmaceutical composition. In some embodiments, the therapeutically effective amount of the pharmaceutical composition comprises TGFb1 at a concentration of about 2.5 ng/ml in the pharmaceutical composition. In some embodiments, the therapeutically effective amount of the pharmaceutical composition comprises PDGFa at a concentration of about 25 ng/ml in the pharmaceutical composition. In some embodiments, the method improves corneal transparency in the subject. In some embodiments, the subject has about 50% or less corneal innervation, about 40% or less corneal innervation, about 30% or less corneal innervation, about 20% or less corneal innervation, about 10% or less corneal innervation, or wherein the subject has no corneal innervation. In some embodiments, the subject suffers from corneal anesthesia. In some embodiments, the corneal anesthesia is congenital corneal anesthesia. In some embodiments, the subject is suffering from neurotrophic keratopathy. In some embodiments, the method results in complete recovery of the corneal epithelium. In some embodiments, the subject is suffering from diabetes.

In another aspect of the current disclosure, methods of improving corneal transparency after corneal injury in a subject in need thereof are provided. In some embodiments, the methods comprise administering a therapeutically effective amount of a pharmaceutical composition comprising ciliary neurotrophic factor (CNTF) and at least one additional trophic factor selected from the group consisting of pleiotrophin (PTN), transforming growth factor beta 1 (TGFb1), and platelet-derived growth factor alpha (PDGFa) to the subject to restore corneal transparency after corneal injury in the subject. In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound. In some embodiments, administration comprises topical ophthalmic administration. In some embodiments, the therapeutically effective amount of the pharmaceutical composition comprises PTN at a concentration of about 12.5 ng/ml in the pharmaceutical composition. In some embodiments, the therapeutically effective amount of the pharmaceutical composition comprises CNTF at a concentration of about 25 ng/ml in the pharmaceutical composition. In some embodiments, the therapeutically effective amount of the pharmaceutical composition comprises TGFb1 at a concentration of about 2.5 ng/ml in the pharmaceutical composition. In some embodiments, the therapeutically effective amount of the pharmaceutical composition comprises PDGFa at a concentration of about 25 ng/ml in the pharmaceutical composition. In some embodiments, the method improves corneal transparency in the subject. In some embodiments, the subject has about 50% or less corneal innervation, about 40% or less corneal innervation, about 30% or less corneal innervation, about 20% or less corneal innervation, about 10% or less corneal innervation, or wherein the subject has no corneal innervation. In some embodiments, the subject suffers from corneal anesthesia. In some embodiments, the corneal anesthesia is congenital corneal anesthesia. In some embodiments, the subject is suffering from neurotrophic keratopathy. In some embodiments, the method results in complete recovery of the corneal epithelium. In some embodiments, the subject is suffering from diabetes.

In an aspect of the current disclosure, methods of improving corneal epithelial renewal in a subject in need thereof are provided. In some embodiments, the methods comprise administering a therapeutically effective amount of a pharmaceutical composition comprising ciliary neurotrophic factor (CNTF) and at least one additional trophic factor selected from the group consisting of pleiotrophin (PTN), transforming growth factor beta 1 (TGFb1), and platelet-derived growth factor alpha (PDGFa) to the subject to improve corneal epithelial renewal in the subject. In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound. In some embodiments, administration comprises topical ophthalmic administration. In some embodiments, the therapeutically effective amount of the pharmaceutical composition comprises PTN at a concentration of about 12.5 ng/ml in the pharmaceutical composition. In some embodiments, the therapeutically effective amount of the pharmaceutical composition comprises CNTF at a concentration of about 25 ng/ml in the pharmaceutical composition. In some embodiments, the therapeutically effective amount of the pharmaceutical composition comprises TGFb1 at a concentration of about 2.5 ng/ml in the pharmaceutical composition. In some embodiments, the therapeutically effective amount of the pharmaceutical composition comprises PDGFa at a concentration of about 25 ng/ml in the pharmaceutical composition. In some embodiments, the method improves corneal transparency in the subject. In some embodiments, the subject has about 50% or less corneal innervation, about 40% or less corneal innervation, about 30% or less corneal innervation, about 20% or less corneal innervation, about 10% or less corneal innervation, or wherein the subject has no corneal innervation. In some embodiments, the subject suffers from corneal anesthesia. In some embodiments, the corneal anesthesia is congenital corneal anesthesia. In some embodiments, the subject is suffering from neurotrophic keratopathy. In some embodiments, the method results in complete recovery of the corneal epithelium. In some embodiments, the subject is suffering from diabetes.

In an aspect of the current disclosure, methods of treating diabetic keratopathy in a subject in need thereof are provided. In some embodiments, the methods comprise administering a therapeutically effective amount of a pharmaceutical composition comprising ciliary neurotrophic factor (CNTF) and at least one additional trophic factor selected from the group consisting of: pleiotrophin (PTN), transforming growth factor beta 1 (TGFb1), and platelet-derived growth factor alpha (PDGFa) to the subject to treat the diabetic keratopathy. In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound. In some embodiments, administration comprises topical ophthalmic administration. In some embodiments, the therapeutically effective amount of the pharmaceutical composition comprises PTN at a concentration of about 12.5 ng/ml in the pharmaceutical composition. In some embodiments, the therapeutically effective amount of the pharmaceutical composition comprises CNTF at a concentration of about 25 ng/ml in the pharmaceutical composition. In some embodiments, the therapeutically effective amount of the pharmaceutical composition comprises TGFb1 at a concentration of about 2.5 ng/ml in the pharmaceutical composition. In some embodiments, the therapeutically effective amount of the pharmaceutical composition comprises PDGFa at a concentration of about 25 ng/ml in the pharmaceutical composition. In some embodiments, the method improves corneal transparency in the subject. In some embodiments, the subject has about 50% or less corneal innervation, about 40% or less corneal innervation, about 30% or less corneal innervation, about 20% or less corneal innervation, about 10% or less corneal innervation, or wherein the subject has no corneal innervation. In some embodiments, the subject suffers from corneal anesthesia. In some embodiments, the corneal anesthesia is congenital corneal anesthesia. In some embodiments, the subject is suffering from neurotrophic keratopathy. In some embodiments, the method results in complete recovery of the corneal epithelium.

In an aspect of the current disclosure, methods of treating corneal complications of diabetes in a subject in need thereof are provided. In some embodiments, the methods comprise administering a therapeutically effective amount of a pharmaceutical composition comprising ciliary neurotrophic factor (CNTF) and at least one additional trophic factor selected from the group consisting of pleiotrophin (PTN), transforming growth factor beta 1 (TGFb1), and platelet-derived growth factor alpha (PDGFa) to the subject to treat corneal complications of diabetes in the subject. In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound. In some embodiments, administration comprises topical ophthalmic administration. In some embodiments, the therapeutically effective amount of the pharmaceutical composition comprises PTN at a concentration of about 12.5 ng/ml in the pharmaceutical composition. In some embodiments, the therapeutically effective amount of the pharmaceutical composition comprises CNTF at a concentration of about 25 ng/ml in the pharmaceutical composition. In some embodiments, the therapeutically effective amount of the pharmaceutical composition comprises TGFb1 at a concentration of about 2.5 ng/ml in the pharmaceutical composition. In some embodiments, the therapeutically effective amount of the pharmaceutical composition comprises PDGFa at a concentration of about 25 ng/ml in the pharmaceutical composition. In some embodiments, the method improves corneal transparency in the subject. In some embodiments, the subject has about 50% or less corneal innervation, about 40% or less corneal innervation, about 30% or less corneal innervation, about 20% or less corneal innervation, about 10% or less corneal innervation, or wherein the subject has no corneal innervation. In some embodiments, the subject suffers from corneal anesthesia. In some embodiments, the corneal anesthesia is congenital corneal anesthesia. In some embodiments, the subject is suffering from neurotrophic keratopathy. In some embodiments, the method results in complete recovery of the corneal epithelium.

In an aspect of the current disclosure, methods of delaying the onset of diabetic keratopathy in a subject in need thereof are provided. In some embodiments, the methods comprise administering a therapeutically effective amount of a pharmaceutical composition comprising ciliary neurotrophic factor (CNTF) and at least one additional trophic factor selected from the group consisting of pleiotrophin (PTN), transforming growth factor beta 1 (TGFb1), and platelet-derived growth factor alpha (PDGFa) to the subject to delay the onset of diabetic keratopathy in the subject. In some embodiments, the pharmaceutical composition comprises: pleiotrophin (PTN); ciliary neurotrophic factor (CNTF); transforming growth factor beta 1 (TGFb1); and platelet-derived growth factor alpha (PDGFa). In some embodiments, the pharmaceutical compositions further comprise at least one pharmaceutically acceptable carrier or excipient. In some embodiments, the composition is formulated for topical ophthalmic administration. In some embodiments, the pharmaceutical compositions further comprise one or more additional active compound. In some embodiments, administration comprises topical ophthalmic administration. In some embodiments, the therapeutically effective amount of the pharmaceutical composition comprises PTN at a concentration of about 12.5 ng/ml in the pharmaceutical composition. In some embodiments, the therapeutically effective amount of the pharmaceutical composition comprises CNTF at a concentration of about 25 ng/ml in the pharmaceutical composition. In some embodiments, the therapeutically effective amount of the pharmaceutical composition comprises TGFb1 at a concentration of about 2.5 ng/ml in the pharmaceutical composition. In some embodiments, the therapeutically effective amount of the pharmaceutical composition comprises PDGFa at a concentration of about 25 ng/ml in the pharmaceutical composition. In some embodiments, the method improves corneal transparency in the subject. In some embodiments, the subject has about 50% or less corneal innervation, about 40% or less corneal innervation, about 30% or less corneal innervation, about 20% or less corneal innervation, about 10% or less corneal innervation, or wherein the subject has no corneal innervation. In some embodiments, the subject suffers from corneal anesthesia. In some embodiments, the corneal anesthesia is congenital corneal anesthesia. In some embodiments, the subject is suffering from neurotrophic keratopathy. In some embodiments, the method results in complete recovery of the corneal epithelium.

### BRIEF DESCRIPTION OF THE FIGURES

FIGS. 1A, 1B, 1C, 1D, and IE. Neurotrophic Keratopathy and Model System: A) In NK, corneal scarring causes permanent blindness. B) Neurotization of human cornea: a sural nerve autograft conducts supratrochlear nerve axons into the denervated cornea. C) Neurotization of rodent cornea: axons from the infraorbital nerve are conducted via nerve autografts to the contralateral denervated cornea.⁷ D) Schematic representation of the distribution of the corneal innervation demonstrating the limbal, subepithelial and sub-basal plexuses situated beneath the basal epithelium, which then form intraepithelial nerve terminals (ENT). Subepithelial nerves form the sub-basal nerve plexus (3) that gives off vertical intraepithelial nerve terminals (4), which terminate in the corneal epithelium (Dawson et al. Cornea and Sclera (2011, Elsevier). E) Self-renewal at limbus, step-by-step differentiation and migration of limbal stem cells towards terminally differentiated epithelial cells at central cornea. The inventors hypothesize that limbal and corneal axons ensheathing SC (orange) support maintenance and renewal of corneal epithelium, although their contribution is yet to be defined (red question marks).

FIG. 2 Cell types residing in the corneal limbus: Limbal stem cells (LSC) (including the sub-population of Gpha2-positive), mesenchymal limbal niche cells (MLNCs), transient amplifying cells (TACs) and Schwann cells (SC) reside in the limbal niche. Clusters were identified using the indicated genes in the computational pipeline; uniform manifold approximation and projection (UMAP), rat, resolution 0.8 from 6800 genes. Cluster percentages indicate proportions of every cell type. Scale bars presented for TACs (ki67) apply for the rest of TACs clusters. Sale bars presented for LSC (tsnpan7) apply for all MLNSs and SC clusters.

FIGS. 3A, 3B, 3C, and 3D. Myelinating and non-myelinating SC are abundant in the limbus. A) In situ hybridization demonstrates enrichment of sox10-positive cells in the corneal limbus. Scale bar: 100 μm. B) Live image of tamoxifen-induced TdTomato-positive fluorescent SC in Sox10-TdT mouse whole eye (upper panel) and part of limbus (magnified in lower panel). Scale bars: 1 μm and 200 μm, respectively. C) Close localization of k15-positive LSC (green) and TdTomato-positive (red) SC, demonstrated by immunofluorescent image of the limbal area of Sox10-TdT mouse cornea (as in B). Scale bars: 100 μm and 50 μm, respectively. D) Immunofluorescent image of whole mount rat cornea, demonstrating non-myelinated bIII-positive (green) and highly densely-myelinated MBP-positive (red) neurons in the limbal area, positive for paranodal protein Caspr (green), together with or red sodium channel NaCh⁺-positive (indicated by asterisks) at the nodes of Ranvier in the left and right panels, respectively. Scale bars: 50 μm-left; 2 μm-right.

FIGS. 4A, 4B, 4C, 4D, 4E, and 4F. Corneal injury causes alteration in gene expression and, potentially, affects paracrine interactions between limbal cell populations. A) Western blot for Substance P, performed on lysates of normal, denervated and neurotized rat whole corneas, detects presence of Substance P in normal and neurotized, but not in denervated corneas. GAPDH is a loading control. B) Transcriptome-based network model of predicted paracrine communication between SC and LSC. Arrows indicate direction of communication. Green nodes indicate the ligands with the highest change in their expression between normal and de-epithelized conditions (24 hours after de-epithelization), as quantitatively represented in C. No changes in Sox2 expression by SC detected following de-epithelization in C. D) Transcriptome-based network model of predicted paracrine communication between SC and MLNCs. Arrows indicate direction of communication. Green nodes indicate the ligands with the highest change in their expression between normal and de-epithelized conditions (24 hours after de-epithelization), as quantitatively represented in E. For C and E black columns represent normal healthy cornea and grey columns. F) Upregulation in expression of the indicated genes in MLNCs 5 days after denervation; *p<0.05; **, p<0.01; ***, p<0.005; ANOVA.

FIGS. 5A, 5B, and 5C. Topical treatment with exogenous trophic factors promotes denervated corneal wound healing. A) Live photos of fluorescein-stained rat denervated de-epithelialized corneas, demonstrating progress in corneal epithelial healing during four days post de-epithelialization comparing vehicle (“veh”, n=4) and the trophic factor mixture (PTN, CNTF, TGFβ1, PDGFα) (“treat”, (n=3)) treated corneas. 0 h indicates corneas condition five days post-denervation immediately after de-epithelization. The factors were topically administrated on the wounded corneas immediately after denervation and every 24 hours. B) Quantitative representation of progress of corneal healing, as in A. Error bars represent standard error; *p<0.05; **p<0.01; ANOVA. C) Live bright field photos of rat corneas as in A 72 h, demonstrate recovery in the factor-treated group but opaque, unevenly-recovered cornea (arrow) in the vehicle-treated group.

FIG. 6 shows the structural and cellular composition of the limbal niche. The corneoscleral limbus contains the Palisades of Vogt and limbal epithelial crypts, forming the limbal niche (or limbus). Limbal stem cells (LSCs) differentiate into transient amplifying cells (TACs) that, subsequently, differentiate to the epithelial cells. LSCs together with TACs, form the basal epithelial cellular layer. LSCs lie in close contact with mesenchymal stromal cells (MSCs), myelinating Schwann cells (SCs) ensheathing Aδ fibers, and non-myelinating SC associated with C fibers. LSC, MSC and SC populations engage in juxtaparacrine molecular cross-talk. The limbal MSCs are attached to the basement membrane, interact with blood vessels, and their projections pass through the basement membrane in contact with LSCs. SCs associated with unmyelinated sensory axons penetrate the basement membrane and extend neurites which terminate in the epithelial surface. Adapted from Feinberg et al. 2023.

FIG. 7 shows clinical images of neurotrophic keratopathy (NK). Clinical appearance of anesthetic corneas, showing central corneal opacity with overlying epitheliopathy. Conjunctival injection, corneal neovascularization, and corneal stromal scar with an overlying epithelial defect are demonstrated. Top right: A cornea with a shallow corneal ulcer. The clinician has applied fluorescein, which indicates the location of the de-epithelialized corneal stroma. Top right: Another patient with a history of recurrent corneal ulceration leading to early opacification of the corneal stroma. Bottom left: A patient with severe NK leading to corneal perforation and infection. Bottom right: A patient with recurrent ulcerations leading to total opacification of the cornea. Feinberg et al. 2023.

FIGS. 8A, 8B, 8C, 8D, and 8E show that ablation of corneal SCs causes the NK phenotype. A) Immunofluorescent image of Sox10-TdT (left panel) and Sox10-TdT;DTA mouse cornea demonstrating abundant bIII-positive axons (green) in the SC-ablated cornea (Sox10-TdT;DTA, right panel), with numbers of axons comparable to those of the non-SC-ablated (Sox10-TdT, left panel) cornea, despite a significant reduction in volume of TdT-positive SCs (red) 10 days post tamoxifen administration, as shown in the magnified lower panel images. Scale bar: 100 μm. B) In vivo photos of mouse corneas demonstrate opacification of the SC-ablated left (L) cornea (arrows), compared to the clear cornea in the non-SC-ablated right (R) eye. C) In vivo photos of deepithelialized mouse corneas demonstrate the progress of corneal epithelial healing for four days post de-epithelialization in SC-ablated (SC abl, Sox10-DTA mice) and SC-present (SC pr) tamoxifen treated corneas. “0 h” indicates the cornea condition immediately after epithelial removal, ten days after tamoxifen administration. Fluorescein (yellow) stains the corneal area lacking epithelium. D,E) Quantitative representation of SC ablation in A (n=3 per condition and the corneal healing in E (n=6 per condition), respectively. Error bars represent standard error; *p<0.05; **p<0.01; ***p<0.005; t-test in D and ANOVA in E.

FIG. 9 shows a predicted model of intercellular communication in rat limbus. Transcriptome-based network model of computationally prognosed paracrine communication between SCs-LSCs and MSCs-LSCs, based on scRNA-Seq analysis of the denervated, intact and de-epithelialized corneas. Arrows indicate direction of communication. Green and yellow nodes indicate the expression of genes encoding for ligands of interest in both SCs and MCSs. Green nodes indicate genes with a notable expression change, while yellow nodes indicate genes with no or minor expression changes between normal, de-epithelialized (24 hours after deepithelization) or denervation (5 days after denervation) conditions. The purple node indicates de-novo expression of Osm by MSCs following corneal denervation of (inj) corneas.

FIGS. 10A, 10B, 10C, 10D, 10D, 10E, and 10F shows NRG1 induces wound healing in dener-vated corneas by preserving SCs. Live fluorescent images of Thy1-YFP (A) and tamoxifen pre-treated Sox10-TdT (B) mouse denervated corneas, acquired prior to (0 h) and at the two indicated time points after corneal denervation. At 0 hours healthy YFP-positive axons (in A) are associated with TdT-positive SCs (in B) (arrowheads). The gradual axonal loss, appearing in form of punctate debris by 48 hours (arrows, top center), completely disappear by 72 hours after denervation (A, top row), is associated with a complete loss of SCs, as reported⁴ (B; middle panel) that is rescued by two daily treatments by NRG1 (B; lower panel; arrowheads). Scale bar in A and B: 1 mm. C) Postfixation immunohistochemical analysis of Sox10-TdT-positive innervated (left image), or 72 hours post-denervated vehicle-only- or NRG1-treated (central and right images, respectively) corneas, as in A. Indicated zoomed-in areas demonstrate presence or absence of bIII-positive (green) epithelial axons in innervated or surgically denervated corneas, respectively (lower panel). The treatment with NRG1 supported survival of TdT-positive SCs in limbus (red; in merged (upper panel) and zoomed-in indicated areas (lower panel)) that, otherwise, degenerated in vehicle-only treated denervated cornea, as indicated by TdT-positive SC cell debris (lower panel; arrowheads). Epithelial and subepithelial cellular layers are indicated by a dense cell dapi-positive nuclei (blue; upper panel). Scale bars: upper panel—200 mm, lower panel—100 mm. D) Preliminary quantitative representation of TdT signal positive areas in limbi of normally innervated (inrv; n=4), denervated NRG1- (n=3) or vehicle-only- (veh; n=4) treated corneas, as in B and C; *p<0.05. ANOVA. E). In vivo photos of deepithelialized rat corneas visualize epithelial healing after denervation compared with normally innervated (inrv) corneas. The corneas were treated either with NRG1 or vehicle-only (veh). Images at “0 h” demonstrates the corneal wound immediately after epithelial removal; fluorescein (green) stains the corneal area lacking epithelium (n=2). F) In vivo photos of rat corneas demonstrate opacification of denervated vehicle-only (veh) treated healed cornea five days after deepithelialization (arrows), compared to a clear NRG1 treated cornea.

FIGS. 11A and 11B shows that SC-derived exogenous topical trophic factors induce healing in SC-ablated, de-epithelialized corneas. A) Live fluorescent images of Sox10-TdT;DTA mouse corneas, 24 h (left panel) and 120 h (right panel) after the first of three consecutive tamoxifen administrations, documenting significant reduction in TdT-positive SC (arrows) in the limbal area⁴. The dashed line separates the central cornea from the limbus (left panel). Scale bar: 200 μm. B) In vivo photos of de-epithelialized mouse corneas demonstrate corneal epithelial healing in SC-ablated (Sox10-DTA mice), compared with vehicle-only (veh) SC-ablated treated, PTN+CNTF treated or wt corneas during 96 hours. “0 h” shows the corneal surface immediately after epithelial removal. Fluorescein (green) stains the corneal area lacking epithelium; n=2/grp. In B (right column), live bright field photos of mouse corneas demonstrate normal recovery in wt corneas and in SC-ablated, factor treated corneas 120 hours following deepithelialization.

FIGS. 12A, 12B, 12C, 12D, and 12E show that SC-derived exogenous topical trophic factors induce healing of denervated deepithelialized corneas. A) Live photos of fluorescein-stained rat denervated deepithelial-ized corneas, demonstrating progress in corneal epithelial healing during 120 hours post de-epithelialization comparing vehicle (“veh”) and the daily trophic factors applied as a combinations of two or single factors. B represent live bright field images 120 hours post-deepithelialization. Normally innervated (inrv) served as positive controls. C) Quantitative representation of corneal healing exposed to the indicated conditions as in A and B, during first 48 hours after deepithelization. Every data point represents a fluorescein negative area per cornea, per condition. D) Quantitative representation of the extent of corneal scarring/opacification, as per indicated conditions in B. The y axle addresses five degrees of the phenotypic severity with 0 referring a completely clear cornea and 5 referring a completely opaque severely scarred cornea, as per the bright field image sample in E. For A and B the number of mice per experimental condition: norm, n=3; veh, n=5; PTN, n=5; CNTF, n=3; PTN+CNTF, n=5); **p<0.01, ***p<0.001, ANOVA.

FIG. 13 shows a cartoon describing the corneal neurotization procedure. A nerve graft is sutured to the divided donor right supraorbital nerve, permitting sensory nerve fibers to regenerate across the nerve graft into the contralateral left denervated cornea. The graft is separated into its component fascicles, which are placed around the limbus. Axons emerge from the graft and grow into the corneal stroma and the overlying epithelium.

FIGS. 14A and 14B show in vivo corneal epithelial lineage tracing. A) Two month-old triple transgenic (Krt(K)15-EGFP;K14CreERT,R26R-Confetti) mice were In vivo corneal epithelial lineage tracing. induced to express Confetti reporters, and limbal removal was performed (yellow arrowheads). For accurate stripe tracking over time, eyes with 1-2 RFP+stripes were followed (n=5 eyes). RFP+ corneal-committed cells repaired the limbus by day 1 and re-expressed K15-GFP in a diffuse pattern (day 10) that became well-defined by day 30. Nasser et al. 20181. B) A whole eye image (as in A) of confetti-positive corneal epithelial clones randomly expressing YFP, CFP or RFP.

FIGS. 15A, 15B, 15C, 15D, 15E, 15F, 15G, 15H, 15I, 15J, and 15K show SC-derived exogenous topical trop-hic factors induce healing of denervated deep-ithelialized corneas. A) Live photos of fluorescein-stained rat denervated de-epithelialized corneas, de-monstrating progress in corneal epithelial healing during four days post de-epithelialization compar-ing vehicle (“veh”, n=4) and the trophic factor mixture (PTN, CNTF, TGFβ1, PDGFα) (“fact-ors”, (n=3)) treated come-as. 0 h indicates corneas condition five days post-denervation, immediately after deepithelialization. The factors were topically applied to the wounded corneas immediately after denervation and every 24 hours. B) Live bright field photos of rat corneas as in A 72 h, demonstrate reco-very in the factor-treated group but opaque, uneve-nly-recovered cornea (arrow) in the vehicle only-treated group. C) Prelimi-nary quantification of corneal healing in A. Error bars represent SE. t-test. Recovery of denervated deepithelialized rat corneas as in A, comparing different combinations of two factors (D-E) and single factors (F-G) applied daily (n=2 per condition). D and F represent live photos of fluorescein staining. Treatment conditions indicated in E and G apply for D and F, respectively. E and G represent live bright field images 120 hours post-deepithelialization. Normally innervated (inrv) and denervated vehicle (veh) treated corneas in D served as positive and negative controls, respectively. H) Immunofluorescent representative image of the limbal area of denervated PTN+CNTF treated cornea of the left (L) eye (lower panel) and normally innervated untreated cornea of right (R) eye (upper panel) demonstrate normal limbal innervation as noted by bIII-tubulin positive axons of the right cornea and a complete absence of innervation in the left denervated cornea. Scale bar: 200 μm. I) Preliminary quantitative representation of corneal healing exposed to the indicated conditions as in D and F, during first 48 hours after deepithelialization. Every data point represents a fluorescein negative area per cornea, per condition. J) Preliminary quantitative representation of the extent of corneal scarring/opacification, as per indicated conditions in E and G. The y axis denotes the degree of opacity with 0 referring to a completely clear cornea and 5 referring a completely opaque severely scarred cornea, as per the bright field image sample in K. For I and J the number of mice per experimental condition: inrv, n=3; veh, n=5; PTN, n=5; CNTF, n=3; PTN+CNTF, n=5); **p<0.01, ***p<0.001, ANOVA.

FIGS. 16A, 16B, and 16C show SC-derived exogenous topical trophic factors induce healing in SC-ablated, de-epithelialized corneas. A) Live fluorescent images of Sox10-TdT;DTA mouse corneas, 24 h (left panel) and 120 h (right panel) after the first of three consecutive tamoxifen administrations, documenting significant reduction in TdT-positive SC (arrows) in the limbal area³. The dashed line separates the central cornea from the limbus (left panel). Scale bar: 200 μm. B, C) In vivo photos of de-epithelialized mouse corneas demonstrate corneal epithelial healing in SC-ablated (Sox10-DTA mice), compared with vehicle-only (veh) SC-ablated treated in B or wt in C corneas during 72 h and 96 h, respectively. “0 h” shows the corneal surface immediately after epithelial removal. Fluorescein (green) stains the corneal area lacking epithelium; n=2/grp. In C, live bright field photos of mouse corneas demonstrate normal recovery in wt corneas and in SC-ablated, factor treated corneas 120 hours following deepithelialization. As expected, NRG1 treatment fails to induce corneas heling, due to DTA-induced ablation of SCs.

FIGS. 17A, 17B, and 17C show an initial analysis of LSC gene expression. A, B) scRNA-seq based quantitative representation of PTPRZ1 (in A) and Notch (in B) in the cluster of LSCs extracted from normal (norm), denervated (den) and deepithelialized (dep) rat corneas³. C) RT-qPCR analysis-based relative representation of changes in mRNA level of ABCG2, Krt15, Krt12 and Krt3 (n=1) genes in three-day cultured hLSC exposed to the indicated compounds-containing Green media⁸ in the concentrations of PTN-50 ng/ml or CNTF-20 ng/ml. The baseline of 1 (100%) represent the gene expression level in un-supplemented Green media. GAPDH transcript was used as reference gene.

FIGS. 18A and 18B show a schematic representation of experimental design.

### DETAILED DESCRIPTION

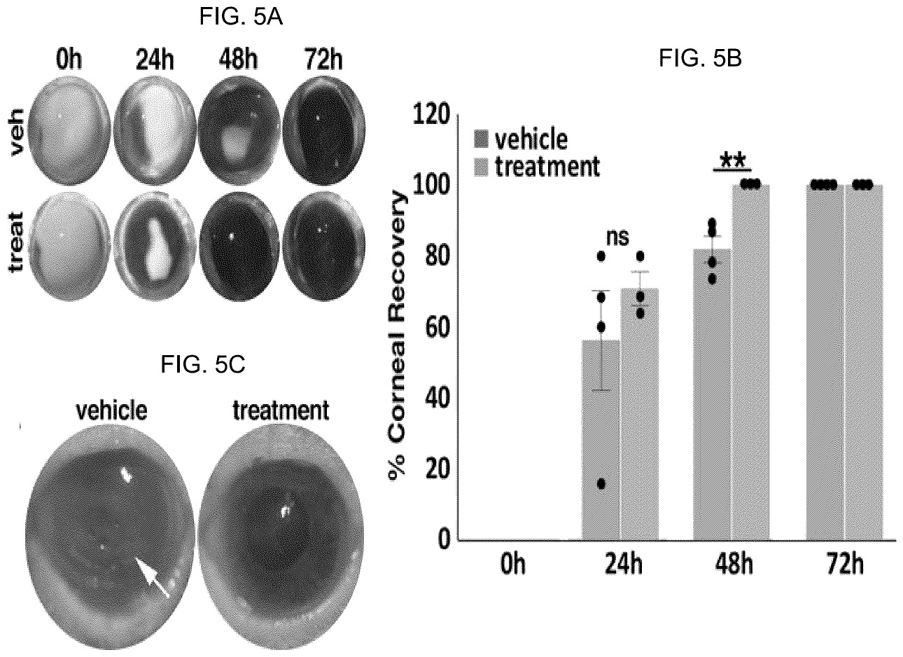

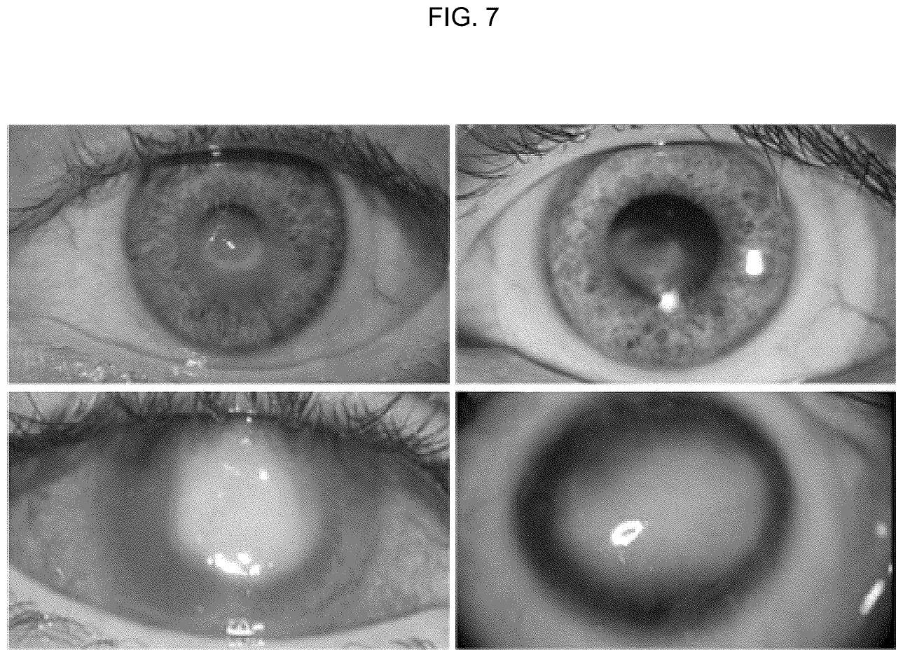

The inventors disclose herein that administration of trophic factors to a subject accelerates healing of denervated corneas in a model of neurotrophic keratopathy (NK) (e.g., FIGS. 7, 17, and 18). Furthermore, the inventors demonstrate that the administration of trophic factors not only improves the speed of corneal healing, but results in a transparent cornea, compared to vehicle treated animals in which the cornea heals, but becomes opaque (e.g., FIGS. 5C, 15J, and 16C).

### Pharmaceutical Compositions

In an aspect of the current disclosure, pharmaceutical compositions are provided. The pharmaceutical compositions comprise trophic factors selected from: ciliary neurotrophic factor (CNTF) pleiotrophin (PTN), transforming growth factor beta 1 (TGFβ1), and platelet-derived growth factor alpha (PDGFα), or any combination thereof. The trophic factors may comprise ciliary neurotrophic factor (CNTF) and at least one additional trophic factor selected from the group consisting of: pleiotrophin (PTN), transforming growth factor beta 1 (TGFβ1), and platelet-derived growth factor alpha (PDGFα). The pharmaceutical compositions may comprise PTN. The pharmaceutical compositions may comprise CNTF. The pharmaceutical compositions may comprise PTN and CNTF. The trophic factors may comprise all four of ciliary neurotrophic factor (CNTF), pleiotrophin (PTN), transforming growth factor beta 1 (TGFβ1), and platelet-derived growth factor alpha (PDGFα).

Ciliary neurotrophic factor (CNTF) may have the amino acid sequence of human ciliary neurotrophic factor (SEQ ID NO: 1). Pleiotrophin (PTN) may have the amino acid sequence of human PTN (SEQ ID NO: 2). Transforming growth factor beta 1 (TGFβ1) may have the amino acid sequence of human TGF1 (SEQ ID NO: 3). Platelet-derived growth factor alpha (PDGFα) may have the amino acid sequence of human PDGFα (SEQ ID NO: 4). Trophic factors comprising or consisting of an amino acid variant or functional fragment that is about 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1-4 are also disclosed herein as useful in present compositions and methods. Fusion polypeptides, comprising or consisting of any combination of SEQ ID NOs: 1-4, variants thereof, or functional fragments thereof are also contemplated herein as useful in the present compositions and methods.

The production of recombinant trophic factors by methods known in the art, e.g., expression in a suitable expression system, e.g., a prokaryotic expression system, a eukaryotic expression system, are known in the art. Further, purification of such recombinant trophic factors, is also considered routine in the art. Thus, one of skill in the art could design a suitable expression and purification systems to generate clinical grade trophic factors for the administration to a subject, e.g., a human subject.

The pharmaceutical compositions may further comprise at least one pharmaceutically acceptable carrier or excipient, examples of which are known in the art. Formulation of pharmaceutical compositions and addition of an appropriate carrier or excipient are considered to be routine activities by one of skill in the art.

The pharmaceutical compositions may further comprise an additional active compound including, but not limited to, an antibiotic, a steroid, an antihistamine, an anesthetic, an analgesic, etc. The additional active compound should, suitably, not interfere with the actions of the trophic factors. The disclosed pharmaceutical compositions may also comprise neuronal growth factor (NGF), e.g., human NGF, which has demonstrated some efficacy in the treatment of NK.

The pharmaceutical compositions may be formulated for any route of administration, including, but not limited to topical ophthalmic administration, topical administration, intraocular administration, intravenous administration, intramuscular administration, intrathecal administration, intracranial administration.

The concentrations of each of the trophic factors may be about 100 pg/μl to about 1000 ng/μl, about 1 ng/μl to about 100 ng/μl, about 100 pg/μl to about 100 ng/μl, or about 100 pg/μl to about 50 ng/μl in the pharmaceutical compositions.

In an exemplary embodiment, the pharmaceutical composition is formulated for topical ophthalmic administration. The concentrations of each of the trophic factors may be about 100 pg/μl to about 1000 ng/μl, about 1 ng/μl to about 100 ng/μl, about 100 pg/μl to about 100 ng/μl, or about 100 pg/μl to about 50 ng/μl in the pharmaceutical compositions formulated for ophthalmic topical administration. The topical ophthalmic formulation may comprise one or more of PTN at a concentration of about 12.5 ng/μl, CNTF at a concentration of about 25 ng/μl, TGFβ1 at a concentration of about 2.5 ng/μl, or PDGFα at a concentration of about 25 ng/μl.

The compositions (i.e., formulations comprising one or more of the disclosed trophic factors, CNTF, PTN, PDGFα, and TGFβ1, and pharmaceutical formulations thereof) described herein may be administered by any means known to those skilled in the art, including, but not limited to, oral, topical, intranasal, intraperitoneal, parenteral, intravenous, intramuscular, subcutaneous, intrathecal, transcutaneous, nasopharyngeal, intra-lesional, intradermal, or transmucosal absorption. Thus, the compositions may be formulated as an ingestible, injectable, topical, ophthalmic topical, or suppository formulation. The compositions may also be delivered within a liposomal or time-release vehicle. Administration of the compositions to a subject in accordance with the invention may exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the composition or compositions being administered, the disease to be treated or inhibited, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological protocol.

The maximal dosage for a subject is the highest dosage that does not cause undesirable or intolerable side effects. The number of variables in regard to an individual treatment regimen is large, and a considerable range of doses is expected. The route of administration will also impact the dosage requirements. It is anticipated that dosages of the compositions will improve the condition being treated by at least 10%, 20%, 30%, 40%, 50%, 60%, 70%, 80%, 90%, 100% or more as compared to no treatment.

The pharmaceutical compositions, including ophthalmic formulations, with the disclosed trophic factors may also include an extended-release vehicle. And an extended-release vehicle may be a biocompatible polymer, dissolved in the carrier or by itself impregnated with the trophic factors to hold the trophic factors and slowly release the drug to the subject, preferably for an extended-release period, e.g., one day, two days, three days, four days, five days, six days, seven days, or more. The biocompatible polymer may be biodegradable or non-biodegradable, depending on desired use and application schedule. Example biocompatible polymers that may be used in the disclosed formulations as an extended-release vehicle include but are not limited to poly-2-hydroxyethylmethacrylate (p-HEMA hydrogels), poly(lactic-co-gly colic) acid (PLGA), polycaprolactone (PCL), hydroxypropyl cellulose, Anecortave acetate (AnA), gelatin, and/or collagen. The inclusion of an extended-release vehicle may, in some cases, allow for less frequent application while still providing effective dosing of the trophic factors.

The terms “pharmaceutically acceptable carrier,” or “excipient” as used herein, mean a non-toxic, inert solid, semi-solid or liquid filler, diluent, encapsulating material or formulation auxiliary of any type. The disclosed pharmaceutical compositions may be formulated for administration by, for example, solid dosing, oral, a topical formulation, injection, inhalation (either through the mouth or the nose), implants, oral, buccal, parenteral, or rectal administration. Techniques and formulations and acceptable pharmaceutically acceptable carriers may generally be found in “Remington's Pharmaceutical Sciences”, (Meade Publishing Co., Easton, Pa.). Therapeutic compositions typically are sterile and stable under the conditions of manufacture and storage.

Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Additionally, pharmaceutically acceptable carriers may be aqueous or non-aqueous solutions, media, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

In some embodiments, the pharmaceutical composition is formulated for local delivery. Suitable additional components for topical delivery are known in the art and include creams, gels, and controlled release drug delivery materials (for example, but not limited to, e.g., PCNU, PGLA, etc.).

Compositions of the present disclosure may include liquids, lyophilized, or otherwise dried formulations and may include diluents of various buffer content (e.g., Tris-HCl, acetate, phosphate), pH and ionic strength, additives such as albumin or gelatin to prevent absorption to surfaces, detergents (e. g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the polypeptide, complexation with metal ions, or incorporation of the material into or onto particulate preparations of polymeric compounds such as polylactic acid, polyglycolic acid, hydrogels, etc., or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The compositions can be sterilized by conventional, well-known sterilization techniques. The compositions may contain pharmaceutically acceptable additional substances as required to approximate physiological conditions such as a pH adjusting and buffering agent, toxicity adjusting agents, such as, sodium acetate, sodium chloride, potassium chloride, calcium chloride, sodium lactate, and the like.

The disclosed trophic factors, or pharmaceutical compositions thereof, described herein may be administered one time or more than one time to the subject to effectively improve the condition being treated, e.g., neurotrophic keratopathy. Suitable dosage ranges are of the order of several hundred micrograms effective ingredient with a range from about 0.01 to 50 mg/kg/day, preferably in the range from about 0.1 to 1 mg/kg/day. Precise amounts of effective ingredient required to be administered depend on the judgment of the practitioner and may be peculiar to each subject. It will be apparent to those of skill in the art that the therapeutically effective amount of the trophic factors described herein will depend, inter alia, upon the administration schedule, whether the composition is administered in combination with other therapeutic agents, the status and health of the recipient, and the therapeutic activity of the particular composition.

### Methods

The inventors have demonstrated that administration of the disclosed trophic factors to animals that have denervated corneas, a model of neurotrophic keratopathy, results in improved corneal healing time and a transparent cornea after healing (e.g., FIGS. 5, 15, and 16). Accordingly, disclosed herein are methods of treating neurotrophic keratopathy (NK) in a subject in need thereof “Treating neurotrophic keratopathy,” as used herein, may comprise one or more of improvement of corneal healing, improved fluorescein exclusion by the cornea, improved epithelial renewal in the cornea, improved transparency of the cornea, or reduction of opacity in the cornea.

In addition, methods of improving corneal healing in a subject in need thereof are provided. Methods of assessing the rate of corneal healing are known in the art and demonstrated in the Examples section and may comprise assaying the cornea with fluorescein for an intact corneal barrier that excludes fluorescence. In contrast, corneas that have not fully healed will appear fluorescent under excitation because they have not excluded fluorescein, see, e.g., FIGS. 5, 15, and 16.

In another aspect of the current disclosure, methods of improving corneal transparency after corneal injury in a subject in need thereof are provided. As discussed above, the inventors discovered that, surprisingly, administration of the disclosed pharmaceutical compositions resulted in a transparent cornea in animals with denervated corneas, in comparison to animals treated with vehicle control which, though the corneas healed, developed opacity during healing. Methods of measuring the overall opacity of the cornea are known in the art and may be performed by a physician, e.g., by visual assessment.

In another aspect of the current disclosure, methods of improving corneal epithelial renewal in a subject in need thereof are provided. Methods of measuring the rate of corneal epithelial renewal are known in the art and may comprise visual assessment, histological assessment of the cornea, or other methods known in the art. Improving corneal epithelial renewal may improve one or more symptom related to reduced corneal epithelial renewal, which is needed for maintenance of the corneal barrier, and may include, but is not limited to, improved corneal wound closure, improved healing of corneal microtraumas, reduced incidence of infection in the eye, e.g., in the setting of NK.

In another aspect of the current disclosure, methods of treating diabetic keratopathy in a subject in need thereof are provided. Diabetic keratopathy, the most frequent clinical condition affecting the human cornea, is a potentially sight threatening condition caused mostly by epithelial disturbances. Diabetic keratopathy exhibits several clinical manifestations, including persistent corneal epithelial erosion, superficial punctate keratopathy, delayed epithelial regeneration, and decreased corneal sensitivity that may lead to compromised visual acuity or permanent vision loss. These features are mainly caused by changes within the composition of the corneal epithelial basement membrane, deposition of glycation products, damage to the corneal nerve endings, reduced tear secretion, and oxidative stress during the hyperglycemic conditions. Clinically, diabetic keratopathy can also appear as punctate keratitis, reduced adherence to the basal membrane, and corneal hypoesthesia.

Thus, as many of the manifestations of diabetic keratopathy are related to lack of corneal epithelial recovery, the disclosed pharmaceutical compositions, which the inventors have demonstrated are effective in improving corneal epithelial renewal and corneal wound healing, are effective in the treatment of diabetic keratopathy. Similarly, the disclosed pharmaceutical compositions treat corneal complications of diabetes, e.g., type 1 or type 2 diabetes, including treating one or more of persistent corneal epithelial erosion, superficial punctate keratopathy, and delayed epithelial regeneration. The disclosed pharmaceutical compositions may improve corneal complications of diabetes or diabetic keratopathy in subjects already afflicted by these conditions or may act to delay the onset or prevent the onset of these conditions.

The disclosed methods may also be described as use of the disclosed pharmaceutical compositions or trophic factors, e.g., PTN, CNTF, TGFβ1, or PDGFα, in methods of, e.g., NK, improving corneal healing in a subject in need thereof, improving corneal transparency in a subject in need thereof, improving corneal epithelial renewal in a subject in need thereof, treating diabetic keratopathy in a subject in need thereof, treating corneal complications of diabetes in a subject in need thereof, or delaying the onset of diabetic keratopathy in a subject in need thereof. Likewise, the disclosed trophic factors may be used in the manufacture of a medicament for treating NK in a subject in need thereof, improving corneal healing in a subject in need thereof, improving corneal transparency in a subject in need thereof, improving corneal epithelial renewal in a subject in need thereof, treating diabetic keratopathy in a subject in need thereof, treating corneal complications of diabetes in a subject in need thereof, or delaying the onset of diabetic keratopathy in a subject in need thereof.

As used herein, “corneal innervation” refers to the density and function of sensory nerve endings in the cornea. Methods of measuring corneal innervation are known in the art and may be performed by, e.g., histology.

As used herein, “corneal anesthesia” refers to a lack of sensation in the cornea. Corneal anesthesia can be a result of a congenital condition, or damage to a branch of the trigeminal nerve which innervates the cornea. Relative sensation in the cornea can be measured by, e.g., Cochet-Bonnet aesthesiometer by one of skill in the art.

As used herein, a “subject” may be any mammal, suitably a human, domesticated animal such as a dog, cat, horse, cow, pig, or a mouse or rat. A “subject in need thereof” is, in some embodiments, a subject diagnosed with a neurotrophic keratopathy, or a subject suffering from lack or corneal innervation, or a subject suspected of developing neurotrophic keratopathy in the future. In some embodiments, a subject in need thereof is a subject who has chronically elevated blood sugar who is at risk of developing diabetic keratopathy. In some embodiments, a subject in need thereof has been diagnosed with type 1 diabetes. In some embodiments, a subject in need thereof has been diagnosed with type 2 diabetes.

In some embodiments, a subject in need thereof is a subject in need of improving corneal healing, e.g., a subject with reduced corneal innervation, which can be detected by assaying corneal sensation in the subject. Relatedly, a subject in need thereof may be a subject in need of improving corneal epithelial renewal.

In some embodiments, a subject in need thereof is a subject that has suffered a corneal injury and has a reduced likelihood of the corneal injury healing without the development of opacity in the cornea.

As used herein the term “effective amount” or “therapeutically effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a subject in need thereof, as defined above.

A “therapeutically effective amount,” also referred to as an “effective amount,” can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; their size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

The effective dosage amounts described herein refer to total amounts administered, that is, if more than one composition is administered, the effective dosage amounts correspond to the total amount administered. Treatment, i.e., the disclosed pharmaceutical compositions, can be administered as a single dose or as divided doses. For example, the treatment may be administered two or more times separated by 4 hours, 6 hours, 8 hours, 12 hours, a day, two days, three days, four days, one week, two weeks, or by three or more weeks. Topical ophthalmic formulations may be administered once daily, twice daily, three times daily, or more, and administration may be performed on consecutive days for a time period prescribed by a physician. Alternatively, administration may be performed for several consecutive days followed by a pause in the administration, whereupon administration may cease entirely or be resumed, as prescribed by a physician.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter.

### Example 1—Limbal Stem Cells (LSCs) Regulate Corneal Wound Healing

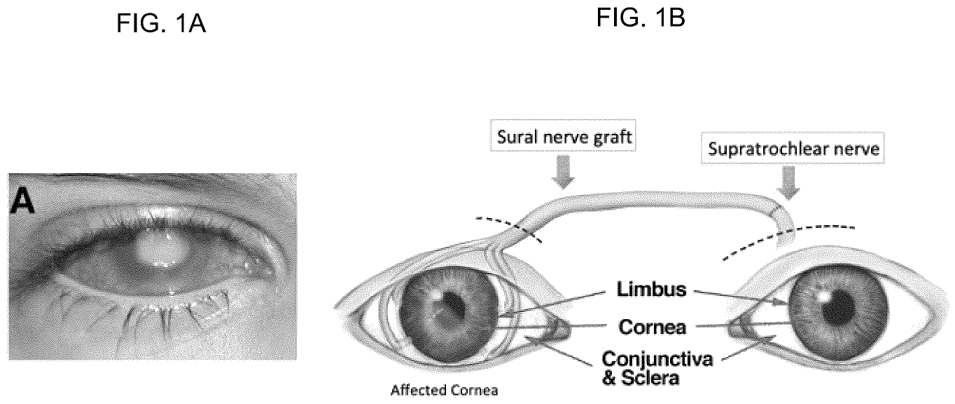

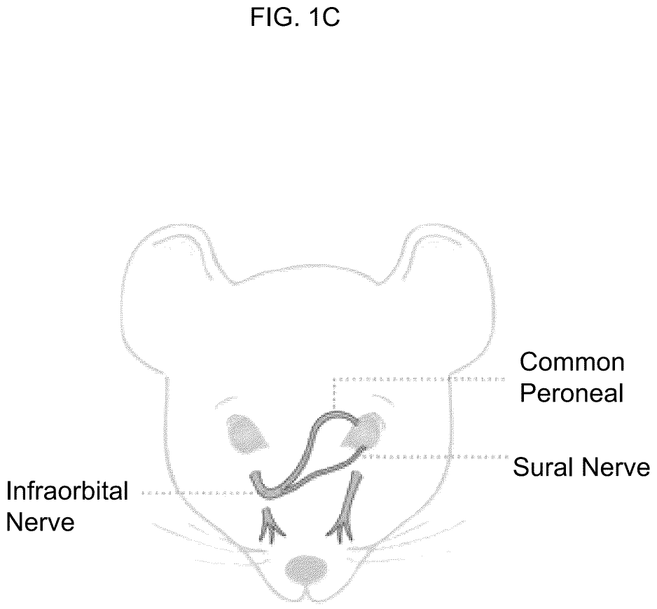

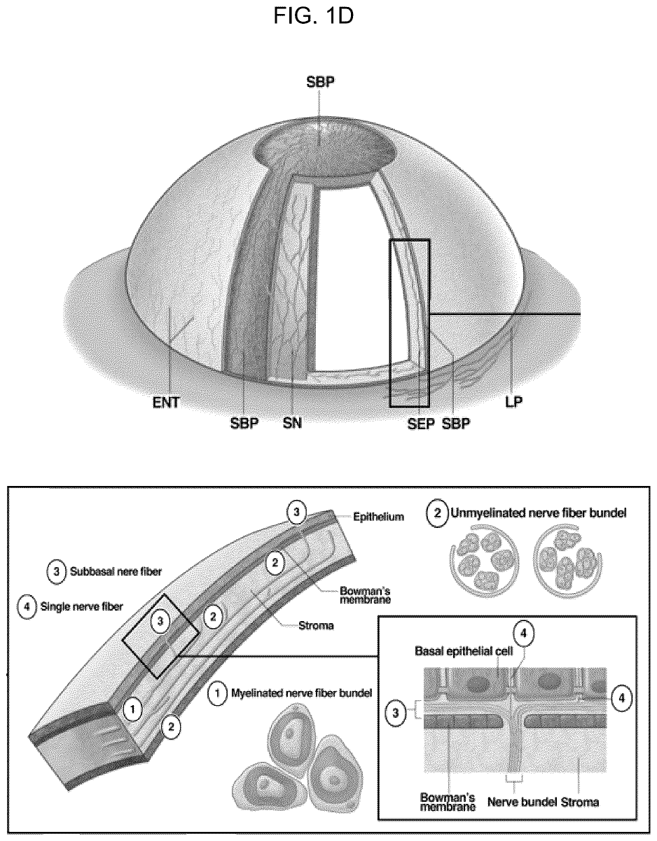

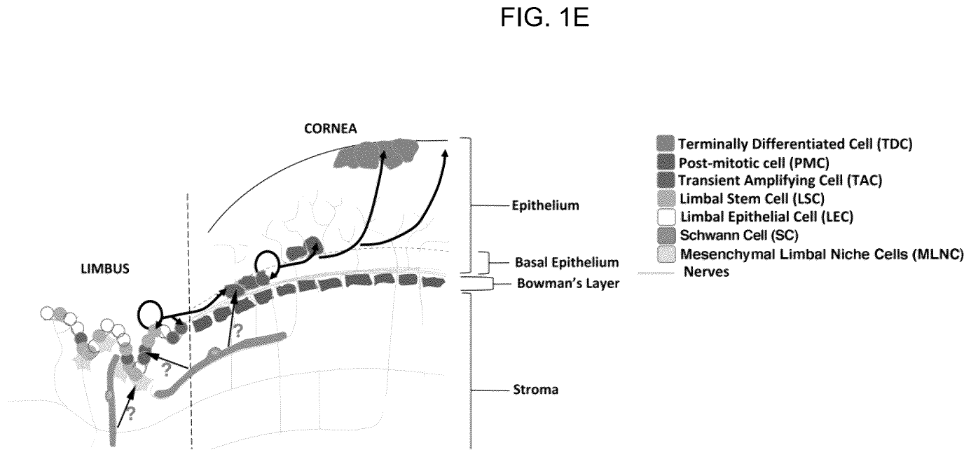

Corneal epithelial maintenance and wound healing^{1,2} depends on limbal stem cell (LSC) activity³⁻⁶. LSC are located in the basal epithelium of the limbus⁵⁸. They interact with limbal mesenchymal cells, which play a critical role in LSC maintenance and activity⁹ (FIG. 1B,D). The LSC progeny differentiate to transient amplifying cells (TACs) that migrate to the central cornea to further differentiate, replenishing the corneal epithelium¹⁰⁻¹⁷. Normally, LSC reside in a growth-arrested or slow-cycling states¹⁸⁻²¹, exhibit morphological characteristics of stem cells, and express genes associated with asymmetric cell division²²⁻²⁶. Corneal clarity is required for vision. Therefore, the cornea, the most densely innervated part of the body, is protected by sensory nerves, responsible for activation of tearing and blink reflexes²⁷. Axons in the basal epithelial layer of the limbus run adjacent to LSC, and their free nerve endings contact epithelial cells (FIG. 1D)²⁸. After corneal injury, LSC progeny give rise to epithelial cells to heal the wound (FIG. 1E). Besides protecting from injury, the sensory nerve fibers likely play a crucial role in LSC proliferation and survival²⁹⁻³² via unknown mechanisms. When protective corneal sensory innervation is lost after infection, trauma, intracranial tumours and ocular surgery, or when innervation fails to develop in congenital cases, permanent blindness results from repetitive microtraumas that induce epithelial breakdown, ulceration, scarring, and opacification of the cornea^{29,31,33,34} (FIG. 1A). This condition, neurotrophic keratopathy (NK), affects nearly 5/10,000 people worldwide^{35,36}. The inventors observed that corneal “neurotization” surgery, using a nerve graft to direct sensory fibers into the denervated cornea in NK patients, can improve corneal wound healing and corneal innervation³⁷. Although not always completely effective in treating NK, the use of this surgical treatment supports the tantalizing hypothesis that corneal epithelial wound healing and maintenance depends on nerves interacting with the LSC population. A clear understanding of innervation-dependent regulation of LSC is required to definitively determine the mechanisms governing corneal wound healing in NK.

Adachi et al suggested that, similarly to skin, corneal oligopotent epithelial stem cells are evenly distributed throughout the ocular surface³⁸. According to this model, during epithelial renewal, corneal epithelial stem cells differentiate vertically to replenish the upper layers of the epithelial cells³⁸. However, more recent reports, using single cell RNA sequencing (scRNA-seq) and lineage tracing approaches in mouse, unequivocally showed that LSC are restricted to the limbal niche, which is also the mostly richly innervated part of the cornea³⁹⁻⁴³. These studies demonstrated a centripetal migration of differentiated LCS progeny during corneal renewal or wound closure in mouse, similarly to human. This conceptual model suggests a unique and different mechanism of regulation of LSC, which are located distantly from the wounded area, unlike in other tissues, such as skin, in which the progenitor cells reside within the wound bed^{44,45}.

Several trophic factors released from corneal sensory axons are thought to regulate corneal epithelial maintenance and healing⁴⁶⁻⁵⁸. Topical recombinant human nerve growth factor (rhNGF) is the only FDA-approved drug for treating NK. However, although, showed no toxicity and inducing a certain improvement in NK symptoms, NGF was not equally efficient in all treated subjects⁵⁹. The authors also did not address the link between greater severity of corneal denervation and worse treatment outcome⁵⁹. Since the mechanism of rhNGF's activity in the cornea is not known, the inventors suggest two interpretations of rhNGF's varying efficacy: (i) NGF alone is insufficient to restore epithelial integrity in cases of complete absence of corneal innervation, and/or (ii) its activity varies depending on other local conditions such as presence/proportion of certain cell populations at the limbal niche or presence/proportion of either of two NGF receptors TrkA and p75^{NTR} reported to be expressed at the basal limbus and known to antagonize each other in other biological systems^{6,61}. Although, NGF is secreted by Schwann cells (SC) in the peripheral nervous system⁶², the sources of corneal NGF and other factors that maintain the corneal epithelium remain unknown.

Therefore, the inventors developed and validated the world's first long-term animal model of NK, achieved by ablation of the corneal sensory innervation modeling the human NK phenotype⁷. A nerve graft was then used (FIG. 1C), which both accelerated corneal wound healing and prevented corneal ulceration⁷. Meanwhile, the inventors' reported that SC are required for wound healing in animal models of fingertip amputations. The inventors hypothesize that the impairment in corneal wound healing is not necessarily due to the lack of perception of sensation resulting in susceptibility to microtraumas, but rather it is the intrinsic inability to maintain and heal the epithelium due to the loss of the SC that are associated with corneal axons.

The inventors intend to (i) determine the role of innervation, particularly SC, on LSC function during innervation-dependent renewal of the corneal epithelium; and (ii) determine the endogenous limbal source of NGF and the role and mode of NGF activity in regulation of the LSC. These novel studies will provide critical understanding of mechanisms of LSC regulation and, in turn, of stem cell biology, in general. In addition, these studies will enable development of new topical therapeutic agents to treat NK^{37,63,64}.

The inventors hypothesize that corneal nerves regulate corneal epithelial renewal via stimulation of LSC. When the inventors neurotized de-nervated corneas in a unique long-term animal model of NK, the inventors observed that neurotization enhanced corneal wound healing and prevented corneal ulceration⁷. Therefore, the inventors are using several approaches (I-IV, below) to test this hypothesis. I. To establish feasibility, the inventors used scRNA-seq of corneal limbus to characterize the cellular composition of the limbal niche. II. In vivo genetic ablation of corneal SC to identify the influence of SC on LSC-dependent corneal wound healing. III. Based on the inventors' previous scRNA-seq studies, to validate predicted ligand-receptor interaction between SC, LSC and the mesenchymal limbal niche cells (MLNCs), the extent to which topical paracrine factors improve corneal wound healing in NK will be determined. IV. Collectively, these approaches will enable determination of the key intercellular signals behind regulation of LSC activity, and will enable the validation of novel topical therapeutic agents in NK.

Determine the role of Schwann cell (SC) stimulation of LSC in corneal wound healing. The uninjured corneal limbus has been well studied⁹, but the cells in the healing cornea and the limbal nerve-associated cells that the inventors hypothesize are crucial to recovery, have not. The inventors hypothesize that limbal cell populations carried by corneal nerves, such as SC, govern corneal epithelial renewal via interaction with LSC. Based on preliminary scRNA-seq analysis the inventors intend to (a) determine the role of SC in LSC activity-dependent corneal epithelial renewal, (b) define SC as a source of NGF in corneal healing, and (c) define the interactions between the limbal cell populations that regulate LSC-mediated corneal epithelial renewal. The inventors hypothesize that Multiple cell types in the limbal niche affect corneal wound healing.

To define the cell types populating the corneal limbal niche (and their expression profiles, as described above), the inventors performed scRNA-seq on harvested dissociated rat limbus, which revealed several ligand-receptor interactions likely to represent paracrine interactions between limbal SC and LSC.

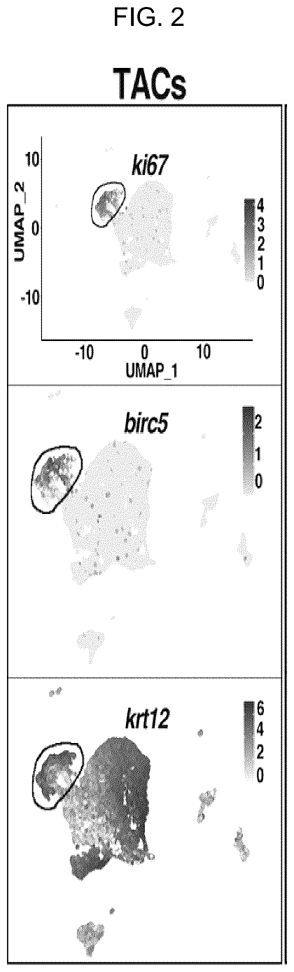

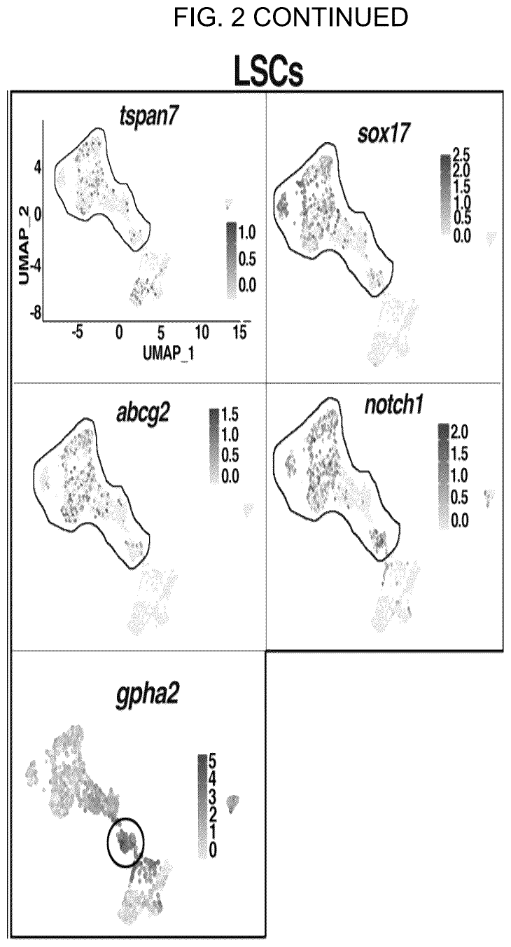

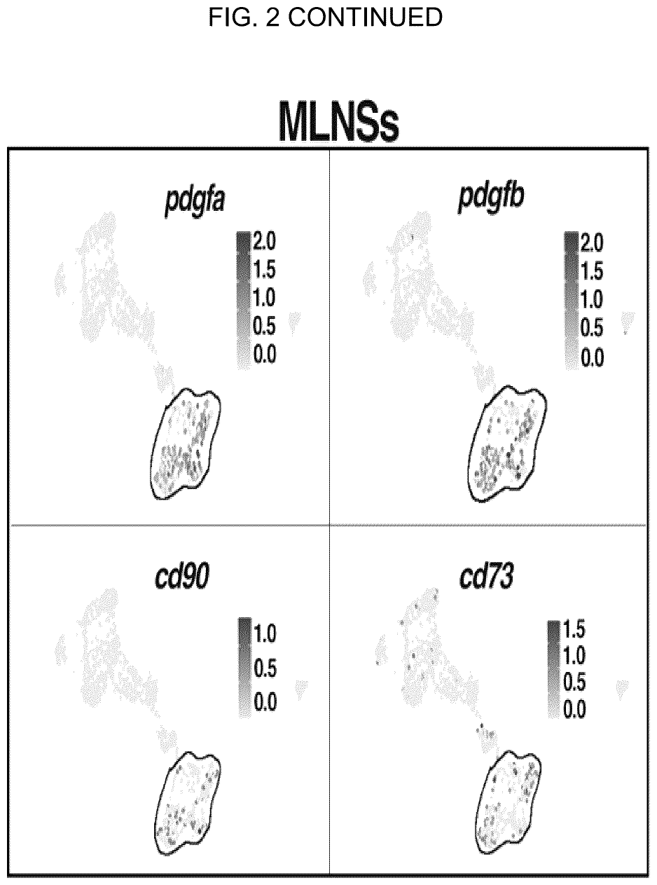

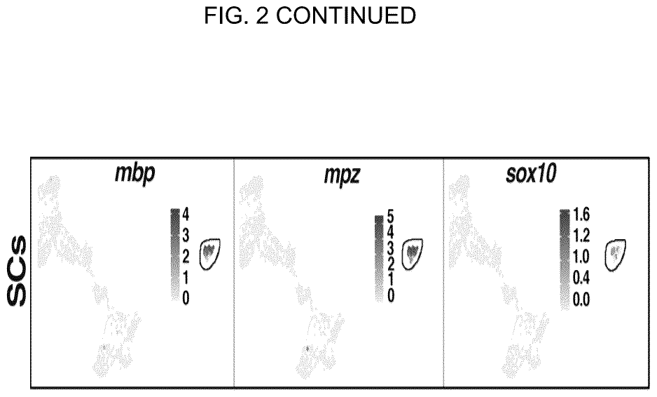

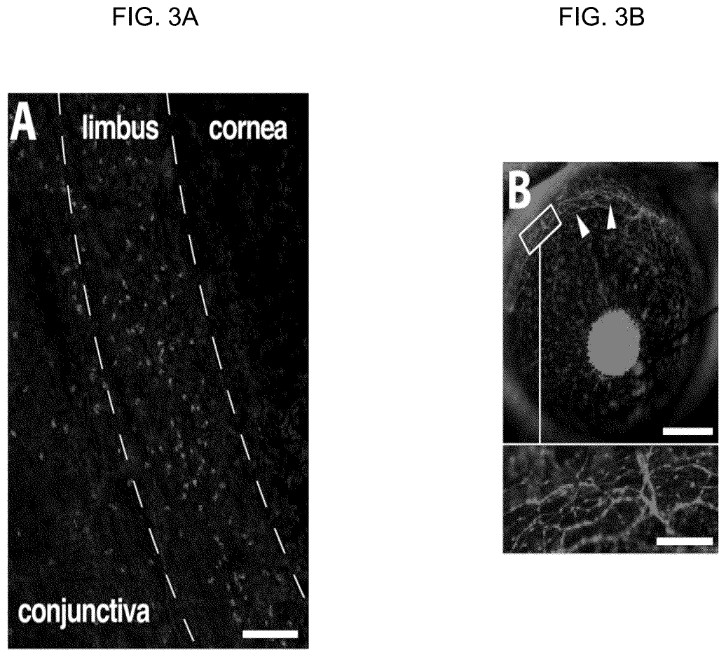

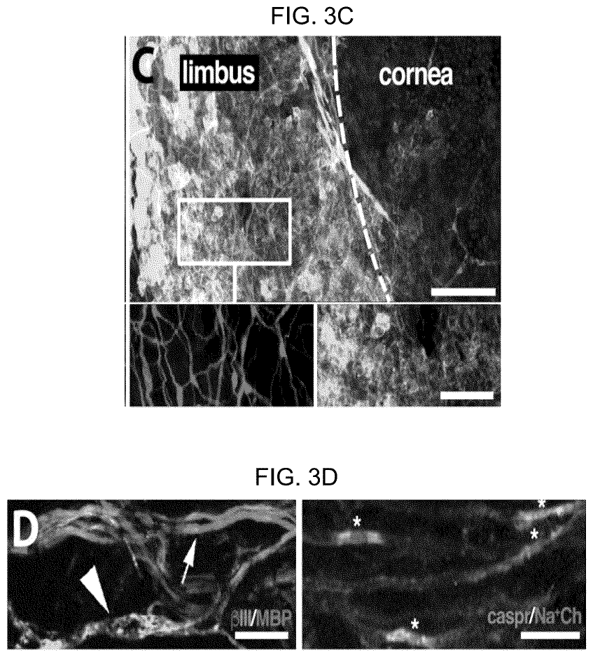

Limbal samples were assessed with high throughput 10× Genomics to identify 10,554-7,000 Median UMI Counts per Cell; 12,000-7,000 cells; with a depth of 4,700-29,000 mean reads/cell. The inventors analyzed the single cell transcriptomes with a unique analysis pipeline that incorporates extensive data quality analysis with visualization and clustering methods, using evidence-based parameter selection⁶⁵. Genes with high variance were used to compute principal components as input to visualize cells in two dimensions with a resolution of 0.8. Clusters of LSC, TACs and MLNCs were detected by a cell population-specific collection of signature genes: ABCG2, C/EBPδ, Notch1, Sox17 and TSPAN7, and the absence of Connexin4 for LSC (including the quiescent sub-population of Gpha2 positive LSC); MBP, MPZ and Sox10 for SC, Birc5 co-localized with the proliferation marker Ki67 and Krt12 for TACs; PDGFRA, PDGFRB, CD73, CD90, and CD105 for MLNCs^{4,5,66-68} (FIG. 2). Further analysis at the higher resolution of 3.2 revealed five sub-populations of LSC, with one sub-population cluster with high Sox17 expression, as well as two MLNC sub-populations, one positive for both PDGFRa and PDGFRb and the other positive for only PDGFRb. Next, based on reports suggesting the SC ensheath the corneal nerves⁶⁹, the inventors validated the SC cluster among the limbal cell populations (FIG. 2). The inventors performed fluorescence in situ hybridization to validate the expression of ABCG2-positive LSC and Sox10-positive SC (FIG. 3A). The inventors also immunostained for SC markers in the mouse model of tamoxifen-inducible CreERT2 the inventors knocked into the terminally differentiated SC-specific promoter Sox10 and the TdTomato reporter gene (sox10-TdT; Table 1). Prior to using this mouse in further experiments, the inventors validated that no spontaneous expression of the transgene occurs and that topical administration of tamoxifen in form of eye drops induces expression of TdT (FIG. 3B). The inventors observed abundant myelinating and non-myelinating SC in the limbus (FIG. 3B,D). These limbal SC were located adjacent to Krt15-positive LSC, and the level of Krt15 expression correlated with the density of SC-associated limbal axons (FIG. 3C).

These observations represent the most comprehensive scRNA-seq analysis of the corneal limbus to date, highlighting the transcriptional heterogeneity of cell populations in the limbal niche. These findings refine the understanding of the cell lineages composing the niche and their intercellular interactions. The inventors further hypothesize that SC regulate corneal maintenance and recovery by directly stimulating LSC. The rationales for this hypothesis are: (i) there is an abundance of SC adjacent to the LCS in the limbus (FIG. 3); (ii) SC control stem cell function and wound healing in other tissues via secretion of ligands^{44,45,70} and (iii) SC secrete NGF, which improves corneal epithelial wound healing^{71,72}.

To determine the extent to which SC are required for corneal maintenance and recovery, SC will be genetically ablated in the cornea. Based on the inventors' observation that denervation-induced loss of SC impairs tissue regeneration in skin and digit tip^{44,45}, the inventors predicted that an analogous SC-mediated process occurs in the cornea. Indeed, the inventors observed that corneal denervation caused complete loss of SC by 48 hours. To further define the specific role of SC in corneal maintenance and wound healing directly, the inventors assessed the effect of corneal SC ablation. Therefore, the inventors generated a Sox10-TdT;DTA mouse in which topical administration of tamoxifen activates Diphtheria toxin (DTA)-induced apoptosis of corneal TdTomato-positive SC. The inventors used a high-resolution fluorescent macroscope attached to a stereotaxic frame to visualize the corneas in vivo. As expected, tamoxifen induced the expression of TdTomato in SC several hours post-Cre activation. As a consequence of diphtheria toxin, the number of TdTomato-positive SC gradually decreased by 58-69% (n=3, p<0.005) by 10 days post-tamoxifen administration. Notably, corneal axons remained intact following SC ablation.

To investigate the effect of SC ablation on corneal recovery, the inventors removed the corneal epithelium with a brush and assessed corneal healing over 4 days. Despite intact corneal innervation and sensation (positive blink response to saline eye drops), SC-ablated corneas failed to heal wounds, like denervated corneas⁷. Healing occurred within 48 hours in control non-SC-ablated corneas of Sox10-Cre tamoxifen-treated littermates. These initial findings demonstrate that presence of SC in limbus is critical for corneal epithelial wound healing and suggests their key role in stimulating LSC during corneal epithelial renewal.

Corneal denervation causes a 75% reduction in cells expressing LSC markers such as ABCG2, p63 and Krt15^{32,73}. To determine whether SC regulate LSC activity, a genetic mouse model will be used to determine if prolonged ablation of SC affects (i) survival of LSC, (ii) recovery of the LSC pool, and (iii) if SC ablation correlates with the denervation-induced phenotype. To answer the first question, Sox10-DTA mouse will be crossed with the keratin 15 (Krt or kl5)-EGFP mouse that constitutively expresses enhanced green fluorescent protein (EGFP) under the LSC-specific complex 1 acidic gene 15 promoter^{40,41} that selectively labels LSC, to obtain the triple mutant Sox10-DTA;kl5-EGFP mouse (Table 1) that will enable visualization of EGFP-positive LSC and assess their number following SC ablation. Induction and maintenance of ablation of SC will be accomplished by weekly topical application of tamoxifen for one month. Following tamoxifen administration, the location and morphology of EGFP-positive LSC in intact and de-epithelialized corneas will be tracked in vivo. The corneas will be immunostained for LSC-specific markers, including ABCG2, p63 and Krt15^{32,73}, as well as the cell proliferation marker Ki67 in harvested corneas at days 10, 20 and 30 post-tamoxifen administration and Ki67/EGFP cells will be localized that are triple-positive. Rigor: In all immunohistochemistry experiments described a minimum of 5 mice per experimental condition will be used. The kl5-EGFP-“only” mice will be used as controls. Using the k15-EGFP mouse, Nasser et al. showed that following physical removal of LSC, differentiated epithelial cells migrate into the limbal niche and de-differentiate to EGFP-expressing LSC to restore the stem cell pool⁴¹. To determine whether SC ablation impedes recovery of the LSC pool following epithelial injury, the Sox10-DTA;kl5-EGFP mouse will be crossed with kl5-cre/PR1, a transgenic mouse that has a synthetic steroid RU486-inducible Cre-mediated recombination system driven by the mouse keratin complex 1, acidic, gene 15 promoter⁷⁴ to obtain Sox10/k15-PR1-DTA;kl5-EGFP mice (Table 1). This mouse will be used to spatially and temporally trace the re-establishment of the EGFP-positive pool of the LSC after sequential ablation of SC and the LSC. Specifically, SC will be ablated with tamoxifen treatment, followed by RU486-induced ablation of kl5-positive LSC one week or one month following the first application of tamoxifen. After cessation of administration of both drugs, re-establishment of the EGFP-positive pool of LSC will be tracked while also assessing recovery of the SC in the cornea. K15-EGFP;kl5-cre/PR1 (SC-non-ablated/LSC-ablated) mice will be used as a positive control.

To determine how SC ablation correlates with the denervation-induced NK phenotype, the inventors assessed the presence of SC after corneal denervation in Sox10-TdT mice. The inventors' initial observations indicate that corneal denervation causes loss of SC by 48 hours and loss of the regenerative capacity beginning 96 hours post-denervation. To assess the effect of corneal denervation on LSC survival in comparison to SC ablation, (a) EGFP-positive LSC in acutely (one week after the first corneal denervation⁷) and chronically (one month after an additional corneal denervation⁷) denervated corneas in k15-EGFP mice will be monitored and (b) neurotization of the acutely and the chronically denervated corneas will be performed and assess ed for the re-appearance of EGFP-positive LSC in kl5-EGFP;kl5-cre/PR1 in mice pre-treated with RU486 (to induce LSC ablation) prior to denervation. To validate the in vivo observations, harvested kl5-EGFP-positive corneas of both experiments (SC-ablated and denervated/neurotized) will be immunolabeled for LSC and proliferation markers as above, and double-positive cells quantified. It is anticipated that: (i) acute ablation of SC will decrease proliferation and/or differentiation of LSC. (ii) Upon withdrawal of the SC-ablative drug, or after neurotization, it is anticipated that anticipate recovery of LSC following reappearance of limbal SC. (iii) A prolonged ablation of SC or corneal denervation will lead to more profound or even irreversible loss of LSC.

Since SC express NGF, the inventors hypothesize that following SC ablation, local deficits in NGF will impair corneal wound healing. To test this hypothesis, the inventors (a) compared the effect of SC ablation (as above) and NGF inactivation on epithelial recovery by treating de-epithelized SC-non-ablated corneas with NGF-neutralizing Ab⁷⁵ and (b) the inventors tested whether topical application of NGF to SC-ablated corneas would rescue corneal healing.

The inventors compared corneal epithelial recovery in two experimental mouse groups: (1) “SC-intact” de-epithelialized corneas treated with daily NGF-neutralizing Ab at 50 ng/ml, and (2) SC-ablated de-epithelialized corneas treated topically with NGF with two administrations per day, at a concentration of 50 mg/ml (the concentration used in a NK clinical trial was 20 mg/ml⁵⁹) comparing them to (3) SC-intact corneas and (4) SC-ablated de-epithelialized untreated corneas. Corneal wound healing was tracked daily over 5 days. The initial results demonstrated that: 1. While NGF neutralization delayed wound healing by 24 hours, epithelialization was nearly complete by day 2. In comparison, SC-ablated corneas showed little recovery after 5 days. This observation suggests that (i) the neutralizing Ab administration protocol did not induce a complete block of NGF (although a block of TrkA tyrosine phosphorylation was seen with acute administration of the Ab (not shown)) or (ii) trophic factors other than NGF secreted by SC are involved in mediating the healing process. 2. Exogenous NGF failed to compensate for SC ablation, and did not improve wound healing that, as in SC-ablated only corneas, reached nearly 60% of the epithelial recovery.

Moreover, unexpectedly, NGF slowed the recovery of the SC-ablated, epithelial-wounded cornea during the first 24 hours. These initial observations suggest that (i) the NGF administration protocol did not provide a sufficient and consistent local concentration of NGF required to improve wound healing, and/or (ii) in the SC-ablated state, as with severe NK cases, NGF treatment is less efficient⁷⁶. The latter concept suggests that trophic factors other than NGF secreted by SC are likely involved in mediating the healing process. It also suggests that near-complete SC ablation, as with prolonged denervation, (i) reduces the sensitivity of the LSC to NGF, or (ii) reduces LSC survival (as suggested above), or (iii) both. The negative effect of NGF on the early stage of the corneal healing suggests that there is more than one mode of action of NGF in the process of the corneal epithelial renewal. The mechanism and role of NGF signaling during corneal epithelial renewal will therefore be addressed. To assess whether administration of NGF or neutralizing antibody is effective, immunohistochemical and western blot analyses will be performed with anti-TrkA/phospho(activated)TrkA, and for the p75NTR effector JNK1/2/phospho(activated)JNK1/2, and if needed modify the inventors' administration protocol based on these experiments.

Following nerve injury in other tissues, mesenchymal cells collaborate with dedifferentiated SC to repair nerve structure and provide a supportive axon c outgrowth environment⁷⁷. The inventors' has also recently reported that mesenchymal precursor cells contribute directly to tissue regeneration⁷⁸. As the MLNC may play a key role in maintaining LSC⁹, SC could be responsible for regulating LSC activity indirectly via MLNC.

Complementary Hypothesis #1: SC regulate survival and activity of MLNC. Mouse genetic models will be used to define the role of SC in MLNC survival and maintenance in vivo. Because MLNC express PDGFRa, the inventors chose to study PDGFRa-EGFP transgenic mice, which robustly express EGFP at the limbus. First, this mouse will be crossed with Sox10-DTA to generate a Sox10-DTA;PDGFRa-EGFP (Table 1) mouse. Ablation of SC will then be induced and the number of EGFP-positive MLNC will be assessed over time. Second, the effect of corneal denervation on survival of the EGFP-positive MLNC will be assessed, comparing the acutely and chronically-denervated cornea (as above) and asking whether LSC number and corneal recovery correlate with the presence of MLNCs. Finally, PDGFRa-DTA;kl5-EGFP (Table 1) mice will be used where MLNC can be specifically ablated and ask whether EGFP-positive LSC survival depends upon the presence of MLNC. SC ablation may reduce the number of MLNC, while ablation of the MLNC, in turn, may affect the survival of LSC. These observations would suggest that SC mediate corneal epithelial maintenance and healing via stimulation of the MLNC.

Complementary Hypothesis #2. SC support axonal regeneration after injury. In this model, the combination of SC and SC-mediated corneal innervation is required for healing. To assess the contribution of SC to corneal innervation, corneal re-innervation in acutely denervated, SC-ablated Sox10-DTA;Thy1 (nerve-specific)-YFP (Table 1) mice using time lapse in vivo imaging will be used as previously described⁴⁵. Results from this experiment would suggest that SC mediate corneal epithelial maintenance and healing by both supporting corneal innervation and stimulating LSC (as above). The inventors hypothesize that multiple intercellular interactions between SC, MLNC and LSC regulate the maintenance and activity of LSC.

The intercellular interactions between various cell populations in the corneal limbus with LSC will be defined by (a) performing a microarray of rat limbus, comparing healthy and denervated corneas and (b) predicting and verifying the expression data obtained from the next generation single cell RNA-seq (as described above).

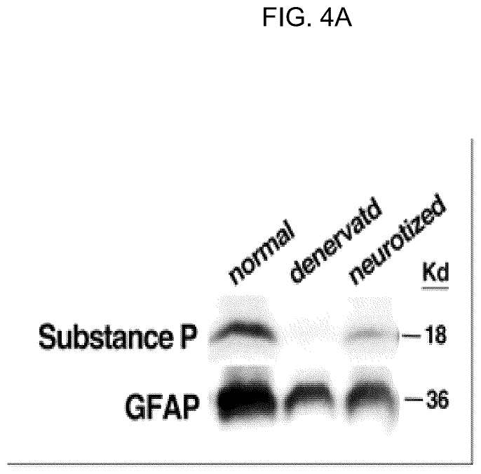

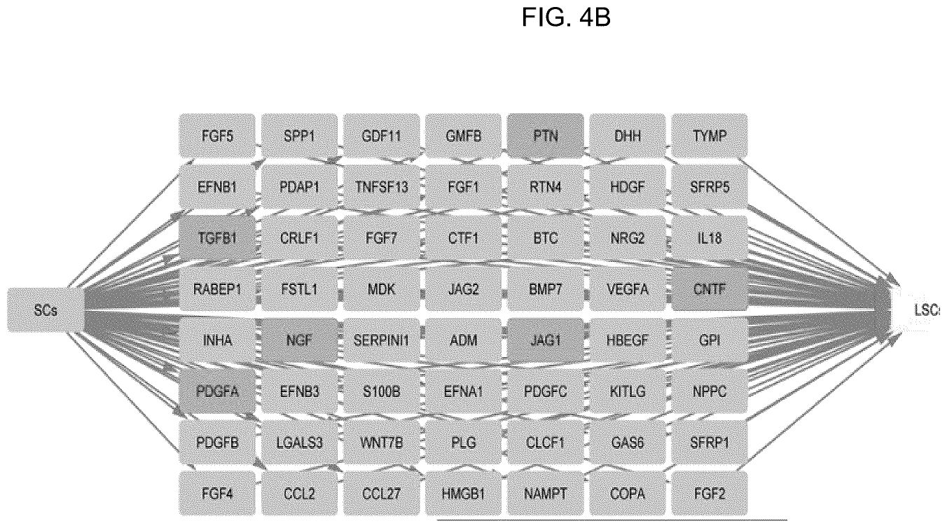

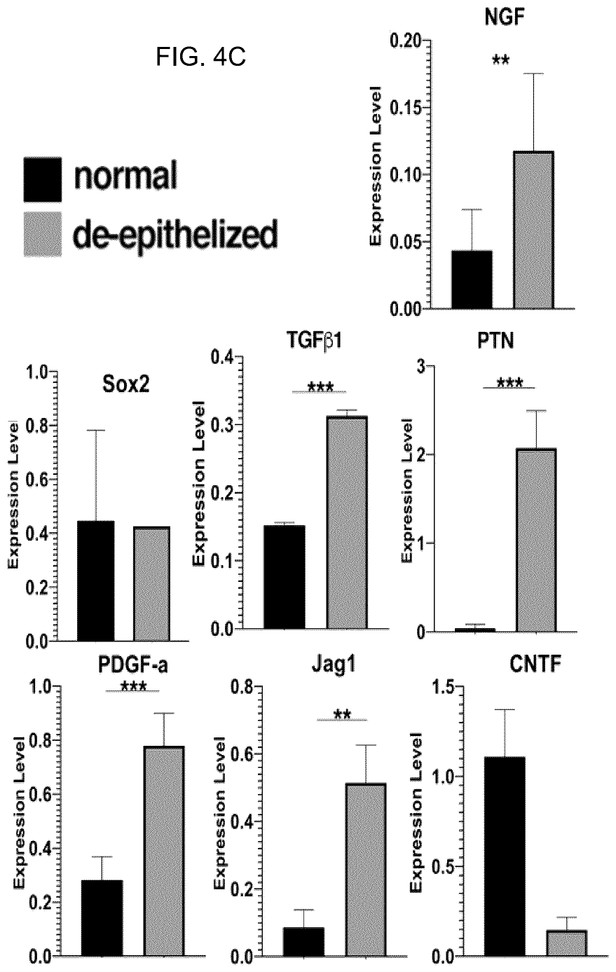

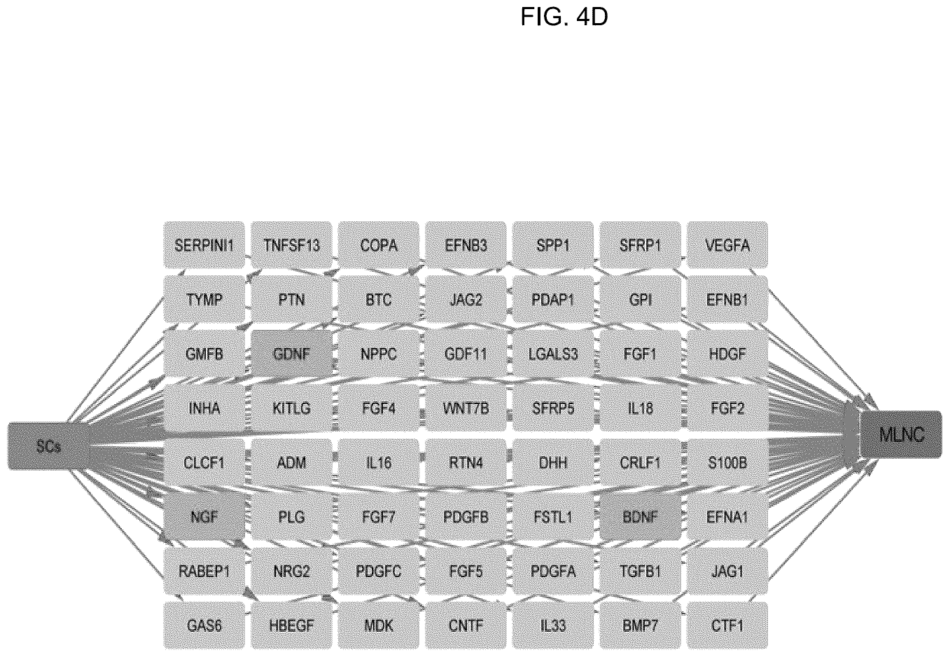

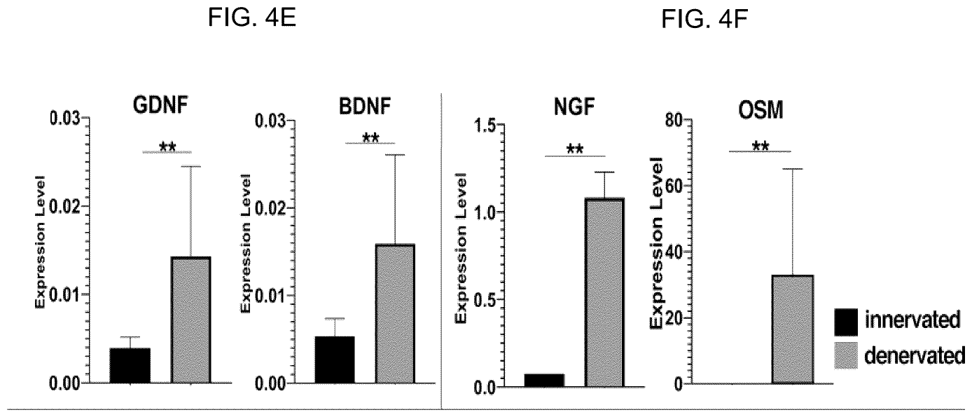

The inventors' initial data from Rat Gene 2.0 ST microarrays comparing RNA from the corneal limbus of normally innervated and denervated corneas demonstrated significant differences (up- or down-regulation) in the expression of 895 genes (0.05<p<2.5×10⁻⁶) one week post-denervation. Additionally, western blot analysis of whole cornea lysates demonstrated complete loss of the neuropeptide Substance P (usually secreted by axon terminals in corneal epithelium) in denervated cornea and its re-appearance in re-innervated cornea following neurotization (FIG. 4A). These initial observations suggest that innervation-dependent signalling maintains corneal surface health. To define the intracellular signalling pathways, the inventors performed scRNA-seq on harvested dissociated rat limbus, comparing normal, de-epithelialized (two biological samples), and denervated (two biological samples) corneas. The inventors extracted potential ligand-receptor interactions using the transcriptome-cell surface communication model approach as the inventors' previously reported 45,79 that suggested communication between SC-LSC and SC-MLNC (FIG. 4B) and relevant downstream intracellular signaling events in LSC. Following de-epithelization, the expression of about 50 genes significantly changed in SC, including upregulation of jag1, tgfb1, ptn, pdgfa and cntf (FIG. 4B,C). Based on previous reports, these genes could potentially play a role in regulation of LSC and following corneal recovery^{9,45,80,81}. The inventors observed that following corneal de-epithelialization, SC kept expressing NGF and started expressing GDNF and BDNF. However, the expression of the receptors for those ligands (including NGF) was no longer detected in LSC, but instead in the MLSC (FIG. 4D,E), suggesting a potential interaction between these two cell population and LSC during epithelial recovery. Following denervation, mesenchymal cells start expressing oncostatin M (OSM) (FIG. 4F), shown together with PDGFa to induce digit tip regeneration⁴⁵. The same pathway may play as critical role in the cornea as it does in the digit tip. However, unlike in skin and digit tip regeneration, where de-differentiated Sox2-positive SC migrate from wounded cutaneous nerves and infiltrate the wound bed to induce regeneration^{44,45}, the inventors observed no up-regulation of Sox2 in SC following corneal de-epithelization (FIG. 4C). Also, unlike in skin and digit tip, corneal LSC are located distantly from the wounded area, and wound closure occurs within 36 hours, while the limbal SC remain with the limbal nerves (FIG. 4B-left). These differences suggest a novel mechanism, where terminally differentiated SC, while ensheathing axons, change their expression profile to regulate adjacent LSC, located distant from the wound site.

To validate the scRNA-seq expression results and identify the locations of the indicated above cell types that express sox2, sox10 along with the candidate ligands and receptors, In situ hybridization (RNAscope) will be performed, supported by qPCR and immunohistochemical analyses performed on harvested normal, de-epithelialized and denervated corneas^{58,74} comparing between corneal central and limbal areas The inventors hypothesize that Trophic factors expressed by SC mediate corneal recovery.

To test for the involvement of multiple trophic factors besides NGF in the healing process, in a gain of function experiment, the inventors performed a single stereotactic electrocautery of the ophthalmomaxillary branch of the trigeminal nerve⁷ to induce an acute corneal denervation. On day five after denervation, the corneal epithelium was removed with a brush. The corneas were treated immediately after denervation and every 24 hours with 10 ml of a mixture containing PTN (12.5 ng/ml), CNTF (25 ng/ml), TGFb1 (2.5 ng/ml), and PDGFa (25 ng/ml) dissolved in PBS, expression of which by the corneal SC was associated with the corneal de-epithelization, as above.

The corneal wound healing of the treated group was compared with the vehicle-treated group daily by fluorescein staining. Surprisingly, combined topical factor application caused complete epithelial wound healing in 48 hours, similar to the healing that occurs in normally innervated, untreated cornea (FIG. 5A,B and not shown). Moreover, the vehicle-treated group's corneas became opaque (probably due to scarring, like the human NK phenotype). However, to the inventors' surprise, the factor-treated corneas were completely transparent and evenly shaped, as expected in a fully-innervated cornea (FIG. 5C). To exclude the possibility of any neuronal survival after the denervation procedure, the inventors performed immunostaining for bIII tubulin, which noted complete absence of nerve fibers in the factor-treated corneas.

For the first time the inventors have demonstrated complete and rapid wound healing in a completely denervated cornea by administering trophic factors to the eye. Having observed that SC disappear from the cornea following denervation, the inventors hypothesized that SC govern corneal epithelial renewal and recovery. Therefore, the inventors applied the factors to denervated corneas to compensate for the loss of SC caused by denervation.

In the future, the inventors plan to: (a) validate the gene expression of the corresponding factors in SC and LSC; (b) determine the extent to which the applied factors compensate for the SC deficiency on wound healing using the inventors' SC-ablation mouse model, described above; (c) define which of the applied factor(s), in isolation or in combination, enhance corneal wound healing; (d) define the extent to which, and how, these factors affect LSC survival and activity. a) As noted above, in situ hybridization, qPCR and immunohistochemistry will be performed to validate the factors encoding genes expression by the corneal SC and their receptors by the LSC. b) In order to demonstrate that the factors are directly associated with the corneal presence of SC, the treatment on normally-innervated, SC-ablated, de-epithelized corneas, using the sox10-DTA mouse will be repeated, while the factors will be applied during the whole course of the experiment, following administration of tamoxifen. c) In order to define which of the four factor(s) induces the corneal recovery, the experiment as above using one of the four factors at a time will be repeated.

Once the active factors are defined, the same experiment using several different concentrations of the active factor(s), with a goal to define the most effective concentration(s) will be repeated. d) SC potentially mediate the corneal epithelial recovery either via supporting LSC survival and/or their activity. The factor(s)-treated corneas (derived from both the denervation and the SC ablation experiments) for LSC markers will be immunostained and cell proliferation markers to define the effect of the treatment on LSC survival and proliferation will be assessed, respectively, when comparing them to the vehicle-treated corneas. It is anticipated that the trophic factor(s) expressed by the corneal SC (and with their receptors expressed by LSC) will compensate for SC ablation by supporting LSC survival and inducing LSC activity. This in vivo experiment will be complemented by examining the effect of the trophic factor(s) on cultured human LSC. A hallmark of LSC is their ability to form long-term proliferative large clones known as holoclones^{85,86}. NGF induces LSC colony formation in vitro⁸⁷.

Human LSC will be harvested as described⁸⁷. Briefly, human limbal rings from cadaveric corneas will be obtained post-mortem under the approval of the local ethics committee. Epithelium will be separated from the underlying stroma following incubation with dispase. Cells seeded at clonal density will be grown in co-culture with J2-NIH3T3 cells growth-arrested with mitomycin C as a feeder layer (40×10³ cells per square centimeter) in epithelial medium, as previously described⁸⁷. LSC potential to survive and make colonies in presence of the defined above as potent trophic factor(s) alone, in presence of NGF or NGF alone will be assessed. A growing media lacking either of the factors will be used as a negative control. If corneal healing is enhanced by multiple trophic factors secreted by SC, it is expected for the maximal LSC colony formation efficiency in presence of all the listed factors, comparing to the newly found factors or NGF alone.

As observed in SC-mediated skin wound healing⁴⁵, more than one factor and/or receptors expressed by LSC may be synergistically involved in regulation of LSC activity (which also will be addressed in Aim 2). Therefore, pairs of the factors will be combined, to test their potential synergistic effect on the corneal recovery and LSC survival and activity. Also, as suggested above, the effect of other factors with upregulated expression as detected by the inventors' computational analysis will be assessed.

The healing experiment described above was performed on acutely denervated cornea, when, unlike in chronically denervated corneas, some residual healing capability still exists. If the discovered trophic factors do not extend LSC survival, but only support the activity of the remaining naturally survived LSC, this treatment will probably not be effective in a chronically anesthetic cornea. To address this possibility, the corneal recovery experiment 8 day after a single denervation and, in later experiments, after a second corneal denervation (as in the inventors' rat NK model)⁷, when no natural recovery occurs will be repeated. In this experiment the treatment (i) started on the day of denervation, (ii) at the time of the corneal de-epithelization, and (iii) negative (vehicle only) control will be compared. The outcome measures in this experiment will be the presence and number of LSC in each of the conditions. Presence and number of MLNC will need to be assessed, as the availability of the limbal niche is a prerequisite for survival of LSC⁹.

In conclusion, the inventors have developed a novel pharmaceutical composition and methods of treatment that, for the first time, have improved corneal healing in the absence of innervation of the cornea.

TABLE 1
Strains used in experiments.

| Strain/Breed | Abbreviation | Description | Goal/usage | n |
|---|---|---|---|---|
| Krt15-EFGP | k15-EGFP | Constitutive | GFP labeling of | 60 |
| | | expression of | LSC | |
| | | EGFP under | | |
| | | Krt15 promotor | | |
| Sox10^{CreERT2/+}; | Sox10-TdT | Inducible | Labeling of | 20 |
| R26-LSL-TdT | | expression of | Schwann cells in | |
| | | TdTomato under | cornea | |
| | | Sox10 promotor | | |
| Sox10^{CreERT2/+}; | Sox10-GT | Inducible | Ablation of | 120 |
| R26-LSL-DTA | | expression of | Schwann cells in | |
| | | Diphtheria toxin | cornea | |
| | | under Sox10 | | |
| | | promotor | | |
| Sox10^{CreERT2/+}; | Sox10-TdT; | Inducible | Track Schwann | 20 |
| R26-LSL-TdT; DTA | DTA | expression of | cells ablation in | |
| | | TdTomato and | cornea | |
| | | Diphtheria toxin | | |
| | | under sox10 | | |
| | | promotor | | |
| Sox10^{CreERT2/+}; | Sox10-DTA; | Inducible | Effect of ablation | 20 |
| R26-LSL-DTA; | k15-EGFP | expression of | of SC on survival | |
| Krt15-EGFP | | Diphtheria toxin | and proliferation | |
| | | under Sox10 | of LSC | |
| | | promotor in k15- | | |
| | | EGFP - positive | | |
| | | mice | | |
| Sox10^{CreERT2/+}/Krt1- | Sox10/ k15- | Inducible | Effect of SC | 40 |
| 15^{cre/PGR}-R26-LSL- | PR1-DTA; | expression of | ablation on | |
| DTA; Krt15-EGFP | k15-EGFP | Diphtheria toxin | recovery of pre- | |
| | | under Sox10 and | ablated EGFP | |
| | | Krt15 promotors, | positive LSC | |
| | | and EGFP under | | |
| | | Krt15 promotor | | |
| Sox10^{CreERT2/+}; | Sox10-DTA; | Inducible | Effect of SC | 40 |
| R26-LSL-DTA; | PDGFRα-EGFP | expression of | ablation on | |
| Pdgfrα^{tm11(EGFP)Sor} | | Diphtheria toxin | survival of | |
| | | under Sox10 | MLNCs | |
| | | promotor and | | |
| | | expression of | | |
| | | EGFP in | | |
| | | PDGFRα | | |
| | | expressing cells | | |
| Pdgfrα^{CreERT2/+}; | PDGFRα-DTA; | Inducible | Effect of MLNC | 40 |
| Krt15-EGFP | k15-EGFP | expression of | ablation on | |
| | | Diphtheria toxin | survival and | |
| | | under | activity of LSC | |
| | | PDGFRα and | | |
| | | EGFP under | | |
| | | Krt15 promotor | | |
| Thy1-YFP | thy1-YFP | Constitutive | Labeling of | 20 |
| | | expression of YFP | neurons and their | |
| | | under Thy1 | axons | |
| | | promotor | | |
| Sox10^{CreERT2/+}; | Sox10-GT; | Inducible | Effect SC | 20 |
| R26-LSL-DTA; | thy1-YFP | expression of | ablation on | |
| Thy1-YFP | | Diphtheria toxin | corneal sensory | |
| | | under sox10 | innervation | |
| | | promotor in Thy1- | | |
| | | YFP - positive | | |
| | | mice | | |

### Example 2—Trophic Factor-Based Topical Treatment for Neurotrophic Keratitis

Neurotrophic Keratopathy (NK) is a major cause of corneal blindness and historically has had no cure. The cornea, the most densely innervated part of the body, is protected by sensory nerves, which activate protective tearing and blink reflexes²⁰. Corneal epithelial renewal^{21,22} depends on the activity of limbal stem cells (LSCs)²³⁻²⁶ located in the basal epithelium of the limbus^{25,27}, where they interact with cells residing in the LSC niche, including corneal nerves(FIG. 1). Besides protecting from injury, corneal innervation likely regulates LSC proliferation and survival²⁸⁻³¹. When protective corneal sensory innervation is absent in NK due to trauma, tumors, infections or congenital causes^{28,30,32,33}, repetitive microtraumas cause corneal breakdown and opacification, inevitably leading to permanent blindness (FIG. 7). To try to treat NK, the inventors' team developed a surgical operation (“corneal neurotization”), in which the inventors direct healthy facial sensory nerves via nerve graft into denervated corneas¹³. Human recombinant nerve growth factor (hrNGF; cenegermin) entered the US market in 2018 to topically treat NK³⁴⁻³⁷. However, both the surgical procedure and cenegermin are ineffective in up to 40% of patients^{12,35,38}. Developing more effective topical therapeutics is therefore required to treat and prevent blindness in NK patients.

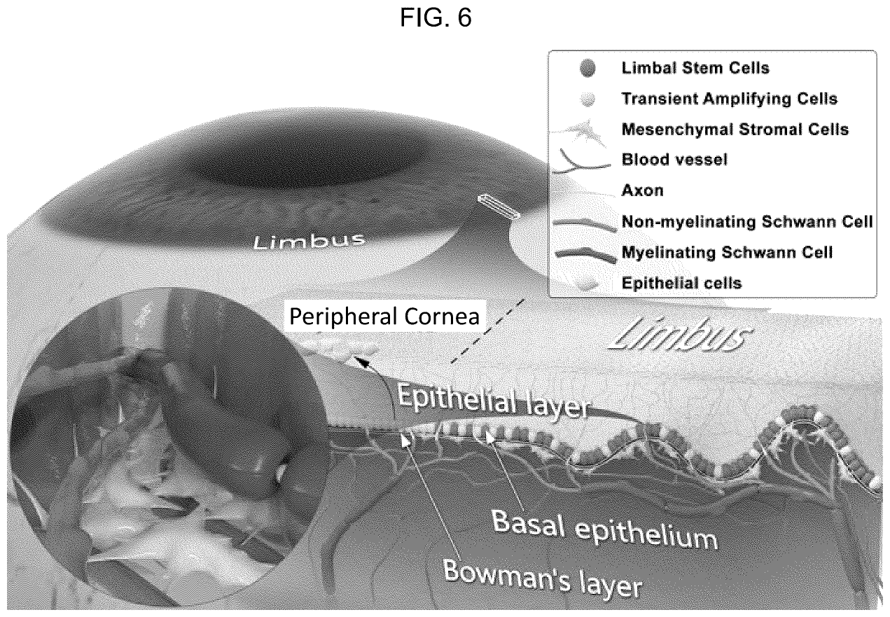

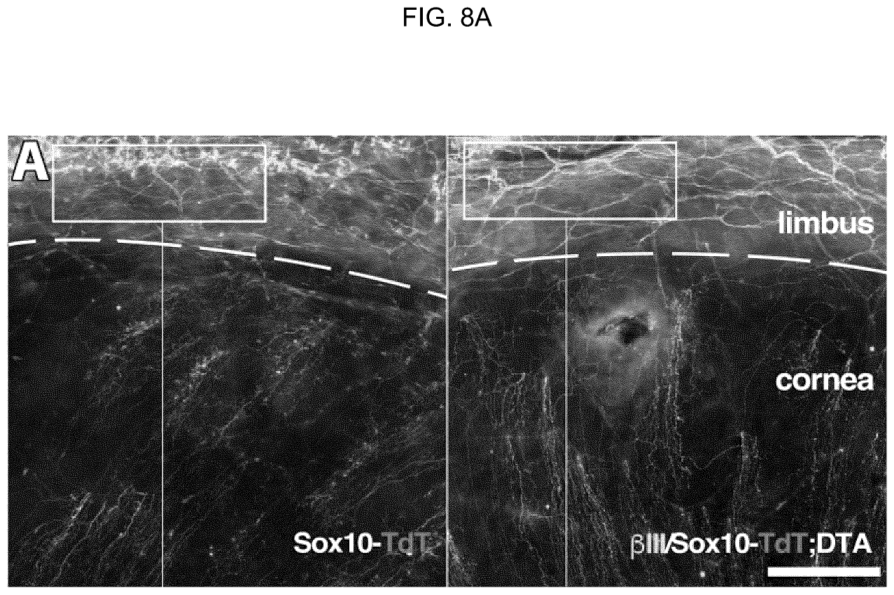

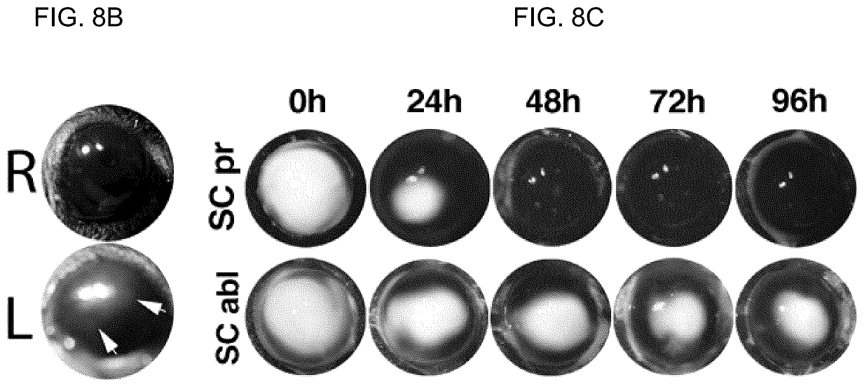

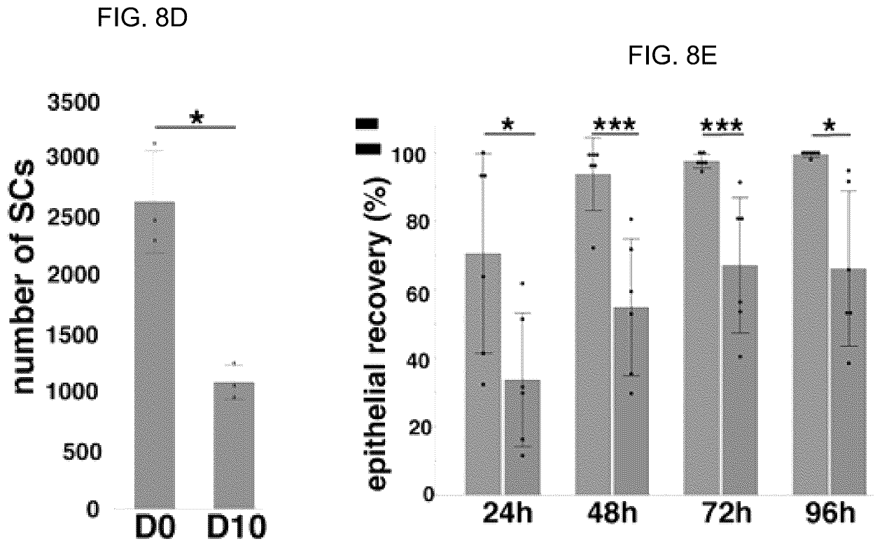

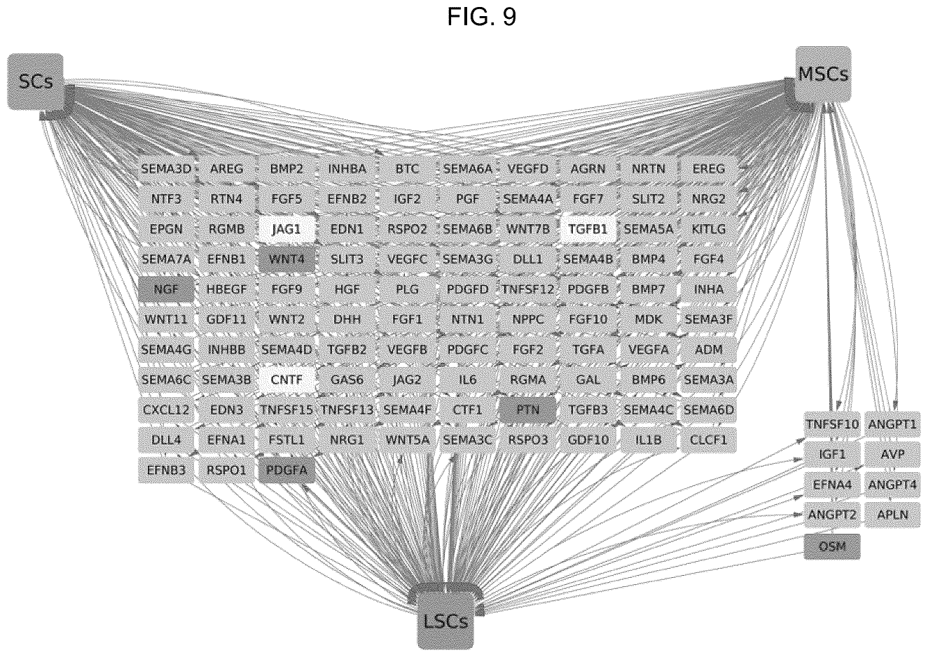

Previously, the inventors observed that corneal axon-ensheathing Schwann cells (SCs) closely associate with LSCs in the limbus (FIG. 6), suggesting that SCs may be involved in corneal epithelial renewal³. Indeed, genetic ablation of corneal SCs induced NK in, otherwise, normally innervated cornea³ (FIG. 8). The inventors used scRNA-seq to model multiple direct and indirect paracrine interactions between SCs and LSCs. These predicted interactions suggested the involvement of known (e.g., NGF), as well as novel trophic factors not previously implicated in corneal epithelial renewal³ (FIG. 9).

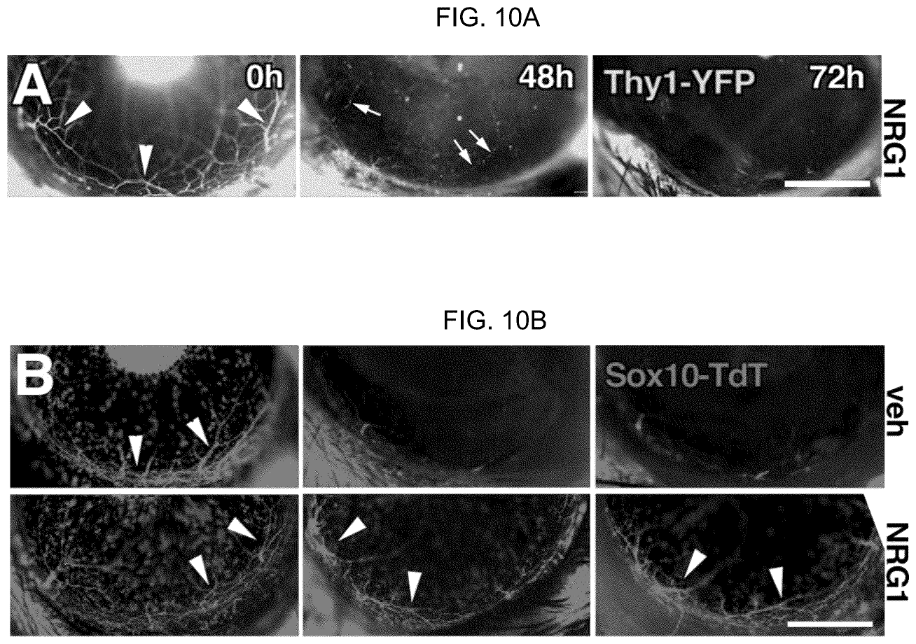

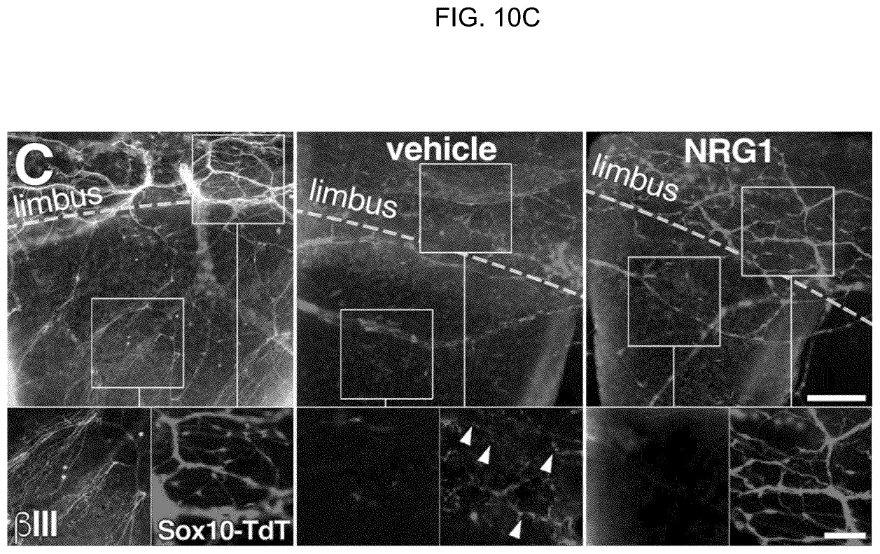

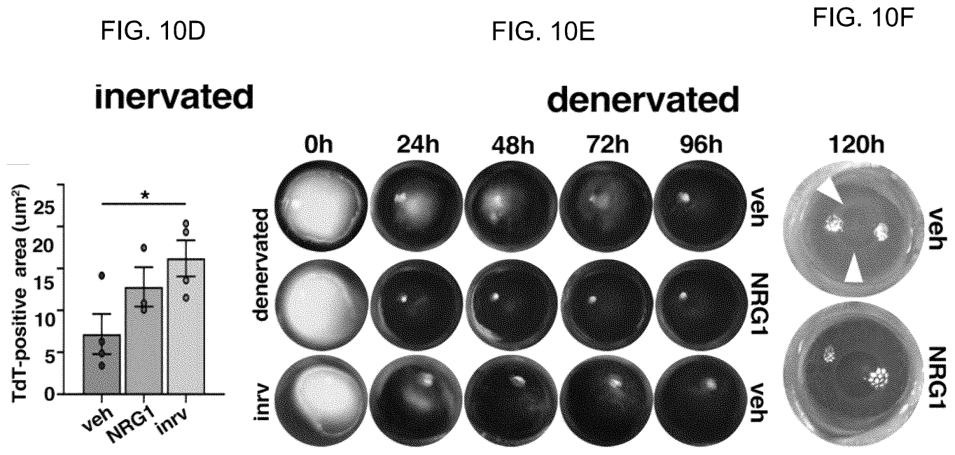

The inventors' recent findings demonstrate the role of SCs in corneal epithelial renewal, and thus prevent NK, in gain-of-function experiments; the inventors used topical daily Neuregulin 1 Type I (NRG1)^{67,68,71} to prevent development of NK in experimentally denervated rodent cornea (FIG. 10). The inventors propose that topical treatment with NRG1 will be indicated when aiming to accelerate recovery and/or developing a preventive treatment of NK for patients in which the innervation to the cornea has been recently disrupted, e.g., due to trauma, tumors, surgery, infections, or other causes, or for which disruption is anticipated.

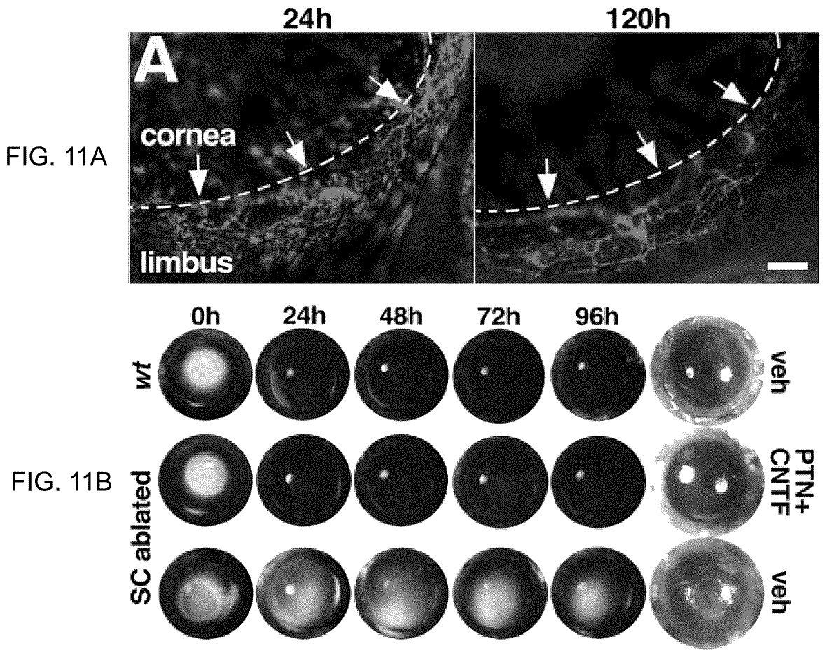

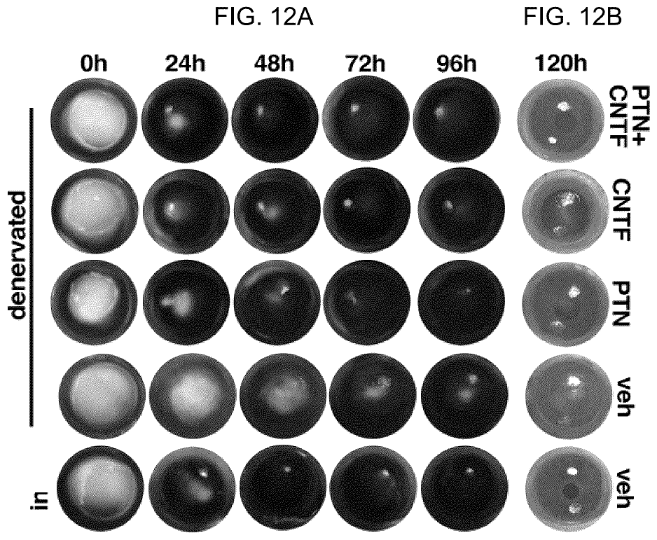

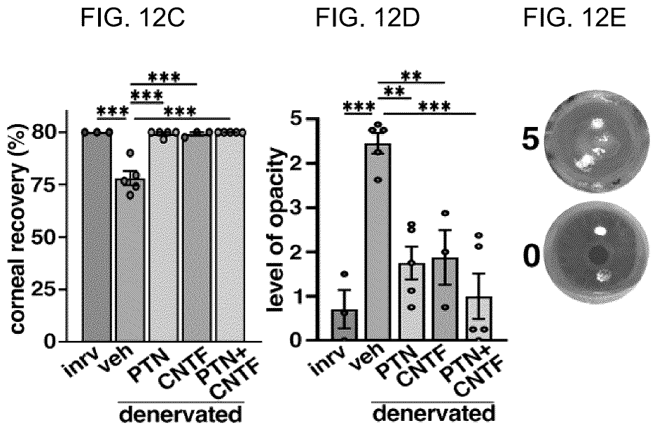

Based on the scRNA-seq-derived model of paracrine interactions, the inventors' new results demonstrate that newly discovered corneal pleiotrophin (PTN) together with previously described ciliary neurotrophic factor (CNTF)⁴⁰ support corneal epithelial renewal, thereby prevent development of NK in experimentally denervated (FIG. 12) or SC ablated (FIG. 11) cornea in rodents.

Based on the inventors' findings, the inventors propose using a novel trophic factor-based topical treatment for different seventies of NK, applied a) alone or b) concomitantly with surgical corneal neurotization, using the inventors' unique rat NK model¹⁵. These therapeutics will target both prevention of ulceration and treatment of corneal wounds in NK.

Collectively, these investigations will elucidate fundamental mechanisms governing corneal wound healing and maintenance and will inform the development of new strategies to treat patients suffering from NK.

### Example 3—Administration of Trophic Factors Treats Neurotrophic Keratitis and Restores Corneal Opacity

### BACKGROUND

Normal Corneal Epithelial Renewal: Corneal clarity is required for vision. Corneal epithelial maintenance and wound healing depend on limbal stem cell (LSC) activity. LSCs are located in the basal epithelium of the limbus and interact with mesenchymal cells, T-cells and other cell types in the LSCs niche. During epithelial maintenance or after injury, LSC progeny differentiate into transient amplifying cells, which migrate toward the central cornea, where they further differentiate to replenish the corneal epithelium (FIG. 6). Normally, LSCs reside in a growth-arrested or slow-cycling state, exhibit morphological characteristics of stem cells, and express genes associated with asymmetric cell division. Axons in the basal epithelial layer of the limbus run adjacent to LSCs, and their free nerve endings contact epithelial cells (FIG. 6). Besides protecting from injury, sensory nerve fibers and their accompanying cells likely play a crucial role in LSC activity and survival and, in turn, epithelial renewal.

Neurotrophic Keratopathy—a Poorly Understood Cause of Corneal Blindness: The cornea, the most densely innervated part of the body, is ordinarily protected by sensory nerves, responsible for protective pain sensation, tearing and blink reflexes. Infections, traumas, intracranial tumours, ocular surgery, or congenital syndromes can impair corneal innervation. In such cases, corneal abrasions and other corneal surface injuries initially go undetected because the patients do not sense pain, leading to corneal opacification and blindness. This condition is termed Neurotrophic Keratopathy (NK; FIG. 7), an otherwise irreversible cause of corneal blindness affecting 5/10,000 people worldwide that historically has had no cure. Unlike in the CNS (for example, retinal ganglion cells), PNS nerves can regenerate, often robustly, offering the chance for a cure for NK.

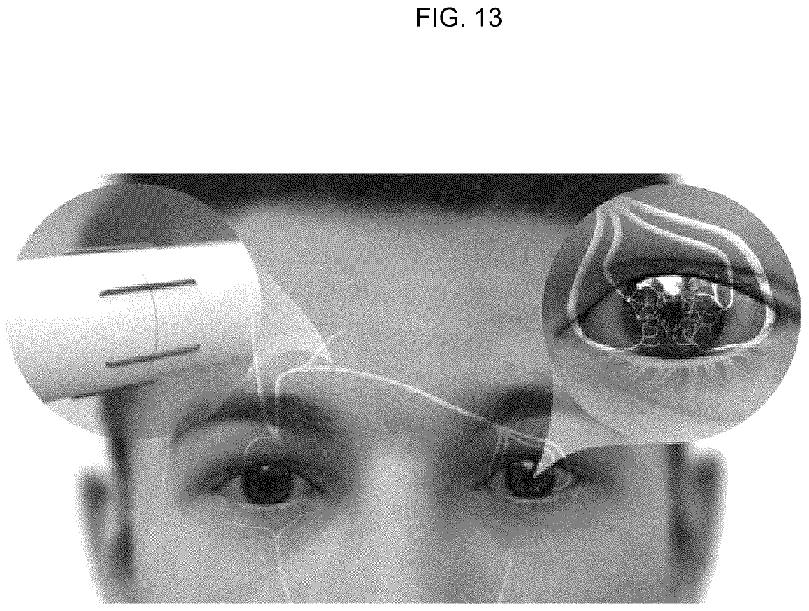

Current Clinical Management of NK: In an attempt to slow the inexorable progression toward corneal opacification and blindness, NK patients are frequently treated with tarsorrhaphy (sewing the eyelids shut to prevent injuries), or are fitted with large custom protective contact lenses. However, neither of these approaches addresses the underlying lack of innervation, and they fail in the long term to prevent NK. To try to induce innervation, clinicians have prescribed recombinant human nerve growth factor eyedrops (rhNGF, marketed as cenegermin). Unfortunately, the treatment of NK was not revolutionized, as was hoped, by the introduction of cenegermin. It is not efficacious in 30% of cases, requires dosing at an extremely high concentration (25 mg/ml) every two hours for months, and treatment is frequently unaffordable for many patients (nearly $100,000 per course). More importantly, the absolute amount of corneal ulcer healing cenegermin induces is relatively low, even in cases of partial corneal denervation. Meanwhile, since 2012, the inventors' group pioneered and others confirmed an approach of treating patients with corneal neurotization surgery, using a nerve graft to direct new sensory fibers into the denervated cornea in NK patients (FIG. 13). This approach improves corneal wound healing and corneal innervation, but 40% of patients fail to gain normal sensation following surgical corneal neurotization, often leading to recurrent ulceration. However, the positive outcomes following corneal neurotization suggest that corneal epithelial wound healing and maintenance depend on cells within nerves, or products of those cells, interacting with LSCs to regulate these epithelial renewal-dependent processes. Critical knowledge gaps in the mechanistic understanding of the process remain, such as: “How, mechanistically, do corneal nerves stimulate LSCs during corneal wound healing and maintenance?” and “Which signals from corneal nerves could become viable therapeutics for NK patients?” Answering these basic questions will enable development of effective topical therapeutics for NK.

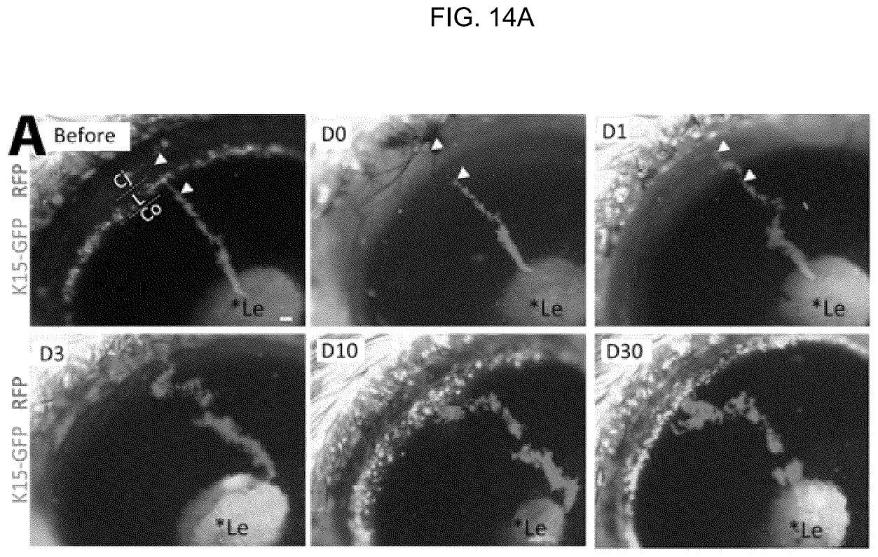

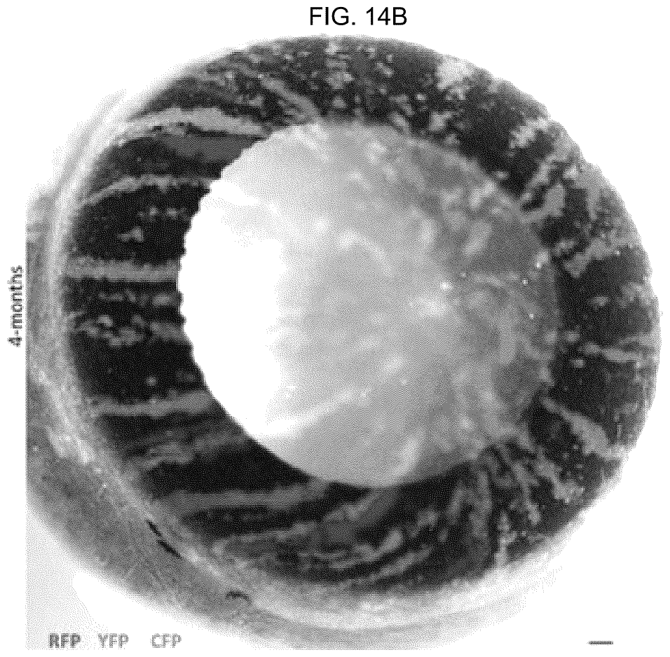

RIGOR OF PRIOR RESEARCH: LSCs are restricted to the limbal niche, the mostly richly innervated part of the cornea. Recent studies demonstrated a centripetal migration of differentiated LSC progeny during homeostatic corneal epithelial renewal or wound closure in rodents and humans (FIG. 14). This conceptual model suggests a unique and different mechanism of regulation of LSCs, which are distant from the wounded area, unlike in other tissues, such as skin, in which the progenitor cells reside within the wound bed itself. The inventors and others have observed that limbal axons are accompanied by myelinating and non-myelinating Schwann cells (SCs), which reside adjacent to LSCs (FIG. 6).

Previously, the inventors reported that corneal nerve-associated SCs regulate corneal epithelial renewal³. The inventors observed that local genetic ablation of corneal SCs induced an NK phenotype and prevented healing of epithelial wounds in otherwise normally innervated corneas (FIG. 8) that maintained their sensation and blink reflexes. Based on the inventors' previous scRNA-seq studies of healthy, de-epithelialized or denervated corneas, the inventors now hypothesize that SCs regulate the activity of the limbal niche via direct and indirect trophic interactions with LSCs. These findings shift the importance away from corneal axons, and towards corneal nerve-associated SCs. Considering the interdependence between SCs and neurons, in the proposed conceptual model the inventors think of corneal axons more as “SC carriers,” rather than direct actors themselves.

GOALS and OVERARCHING HYPOTHESIS: The inventors' overarching goals are: (i) to determine the mechanisms by which SCs regulate LSCs; and (ii) to develop new, non-invasive therapeutics to treat NK. The inventors' preliminary findings (i) suggest that ciliary neurotrophic factor (CNTF) together with, novel in the context of cornea, pleiotrophin (PTN), are secreted by corneal SCs and (ii) demonstrate that these two factors induce corneal innervation-dependent epithelial renewal by directly stimulating LSCs. PTN and CNTF are key regulators of corneal epithelial renewal that act via regulation of LSC maintenance and differentiation.

INNOVATION: Innovations in this proposal include: a) the novelty of the unique corneal neurotization rodent model developed in the inventors' laboratory, which unequivocally defines the critical role of the sensory innervation in the corneal epithelial renewal, b) a novel approach leveraging complementary surgical, molecular and genetic techniques that allow observation of the corneal limbus as a whole, followed by defining the transcriptional events specific to corneal epithelial renewal, c) the novel implication of SCs as a key regulators of LSC, and d) the novelty of defining the SC-expressed trophic factors that directly stimulate LSC during corneal epithelial renewal. These novel studies will provide critical understanding of the mechanisms governing corneal epithelial renewal. Further, the ability to directly visualize stem cell function in real time in the corneal model provides an unusual opportunity to gain mechanistic insights into stem cell biology in general. In addition, these studies will enable development of novel topical therapeutics needed to treat NK, which lacks robust topical treatments.

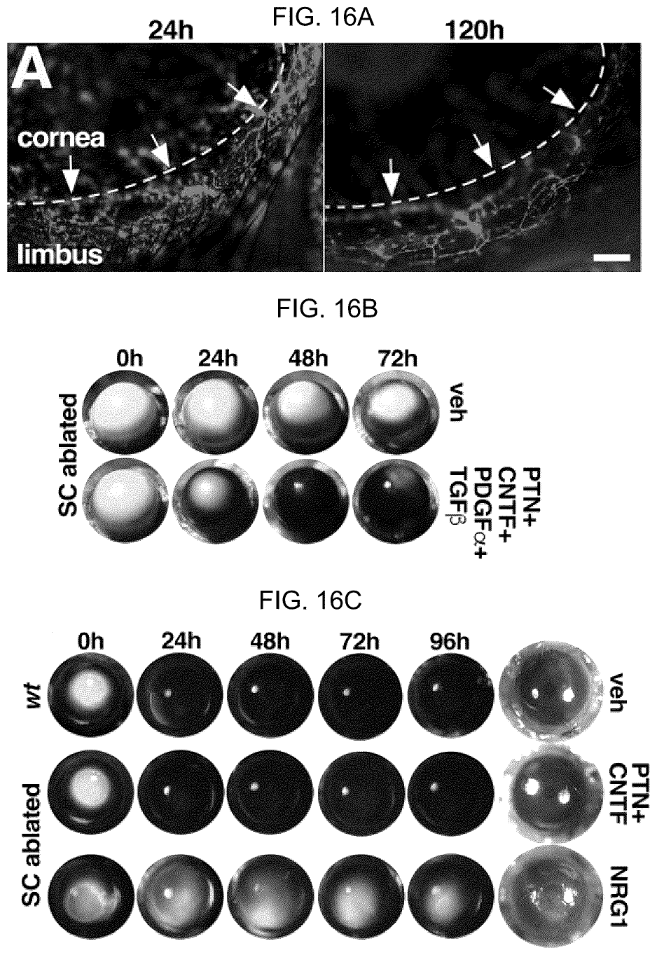

APPROACHES: The inventors have previously demonstrated the critical role of sensory innervation in corneal epithelial wound healing by (i) using a long term surgical corneal denervation model to induce NK in rodents and (ii) treating NK by surgically introducing donor nerves into anesthetic corneas (corneal neurotization) in rodents and humans (FIG. 13). Later, in loss-of-function studies in rodents, the inventors observed that surgically denervating corneas caused rapid withdrawal of limbal SCs (FIG. 10). Moreover, the inventors observed that local genetic ablation of corneal SCs also caused an NK-like phenotype (FIG. 16) in otherwise normally innervated corneas³. Informed by these studies, the inventors' subsequent scRNA-seq analysis of dissociated rat limbi proffered a mechanistic model of paracrine interactions between SCs and LSCs (FIG. 9). Therefore, to define the mechanisms and the roles of SCs in corneal epithelial renewal, the inventors will apply the following approaches.

The inventors will further validate the inventors' prior loss-of-function studies³ with gain-of-function studies. Following surgical corneal denervation, the inventors will topically apply recombinant glia-trophic factor Neuregulin 1 Type I (NRG1) to maintain the viability of corneal SCs and, in turn, maintain the wound healing capacity of denervated corneas, further defining the role of corneal SCs in regulating the epithelial renewal in vivo.

To define SCs as regulators of the LSC niche, the inventors will track the location and activity of LSCs, and assess the extent to which the LSC pool can be restored in denervated or genetically SC-ablated corneas. As above, the loss-of-function studies will be complemented with gain-of-function approaches by treating denervated corneas with NRG1 to rescue SCs and, in turn, determine the extent to which corneal SCs affect LSC survival and activity in vivo.

To determine the mechanisms of SC-LSC juxtaparacrine regulatory communication, leveraging the inventors' published scRNA-seq datasets, the inventors will determine the extent to which topical CNTF and PTN improves recovery of denervated or genetically SC-ablated de-epithelialized corneas in vivo.

To correlate the observations from animal models to humans, and to inform possible future human clinical studies, the inventors will assess how the selected trophic factors affect primary human (h) LSC clonogenicity and expression patterns in vitro, as well as will identify the LSC receptor(s) for PTN.

The inventors will assess the extent to which the topical application of selected trophic factors, either alone or combined with corneal neurotization, improves NK outcomes in the inventors' rodent NK model in vivo.

Rigor, Transparency & Reproducibility: Randomization will be performed while assigning treatment groups for rodent studies using computer-based algorithms. All analyses will be performed double-blinded i.e., data from control vs test samples (masked from laboratory study team) will be handed over to an independent biostatistician. Likewise, for in vitro studies, personnel will submit blinded data to the inventors' biostatistician. As needed, blinded assessment by a third-party evaluator will be used. Complimentary and redundant approaches will be used in experimental designs to ensure rigor.

Data Analysis: Generalized Linear Models (GLM) will be used to analyze the data for all aims using an ANOVA approach. The outcomes for each experiment will be continuous variables. GLM models will enable analysis of Normally distributed data across treatment groups, for any Exponential family distribution, including binary in the event the inventors need to dichotomize the outcomes. For repeated measures when the inventors assess the animals at multiple times, similar analyses using Generalized Estimating Equations (GEEs) will be used to account for the within-animal covariance structure. All analytic assumptions will be verified, and analyses will be performed using SAS v9.4 (SAS Institute, Cary, NC). Power Analysis: Preliminary studies indicate that six animals per treatment group is adequate to find significant associations. For comparisons with two groups, using a two-sided t-test, this indicates that an effect size of 1.80 is necessary to be powered at 80% at an alpha level of 0.05, similar to what has been found previously, and for a four level ANOVA test, the estimated overall effect size for the omnibus test is 0.56, giving an estimated effect size for Bonferroni-corrected pairwise comparisons of 1.82. All initial results presented in this proposal that are based on less than three biological samples (n<3) are addressed as indications and will be statistically validated per the above statistical criteria. Animals: This research proposal is mostly based on in vivo animal studies. Due to surgical procedures challenges, associated with a mouse body size, when no genetic aberrations are involved, the proposed in vivo experiments will be performed on rats. AGE: Rodent experiments will be conducted using adult animals between age 12-16 weeks. SEX: The inventors' previous studies indicate no differences between male and female rodents (rats or mice) in the proposed experiments. Therefore, animals of both sexes will be used for the experiments, based on their age and genetic background. With the inventors' biostatisticians, the inventors will address potential biological variability by disaggregating data into male and female groups and performing post-hoc analysis to look for sex differences. Biological variables: During the proposed experiments the inventors will assess and measure (i) size of corneal epithelial wound, defined by fluorescein-stained area, and/or (ii) corneal scarring, defined by the opaque area of the corneal surface, and/or (iii) the number and/or size of cells and/or cell colonies, based on either in vivo or in vitro fluorescent labeling studies.

AIM 1: Determine the role of Schwann cells as key mediators of corneal innervation dependent epithelial renewal. The inventors' loss-of-function studies showing that local genetic ablation of corneal SCs caused NK in rodents³, suggesting that SCs are a necessary trophic regulator of the epithelial renewal. Therefore, in Aim 1 the inventors hypothesize that limbal SCs govern innervation-dependent corneal epithelial renewal by supporting LSCs and the LSC niche's activity. The inventors will (a) define the role of SCs in corneal epithelial renewal in a novel gain-of-function experiment, (b) define the role of SCs in LSC maintenance and activity using both loss- and gain-of-function experiments, and (c) define the role of SCs in maintenance of the LSC niche in rodents. Clinical relevance: As a novel therapeutic target in NK, topical NRG1 may preserve the viability of SCs and, in turn, prevent corneal ulceration and its complications in newly acquired cases of human NK.

Sub-Aim 1a: To define the effect of NRG1-supported SC survival on recovery of denervated de-epithelialized corneas. 1a.1. Working Hypothesis: Corneal epithelial maintenance and healing after injury depend on SCs. 1a.1.1. Approach: To further define the role of SCs in corneal wound healing, in a novel gain-of-function experiment and correlate between SC survival and epithelial renewal capacity, the inventors will apply topical NRG1 to surgically-denervated rodent (rat or mouse) corneas in vivo. 1a.1.2. Experimental Design and Preliminary Results: In the peripheral nervous system, neuronal paracrine NRG1 supports survival of SCs. Following sensory denervation, the cornea gradually loses its regenerative capacity, and epithelial instability coincides with withdrawal of SCs. The inventors observed that denervated, de-epithelialized rodent corneas exhibit delayed wound closure, accompanied by opacification (FIG. 10). Previously, the inventors and others used NRG1 to grow primary rodent SCs in vitro. To test the extent to which exogenous NRG1 prevents SC loss following denervation, as before, the inventors will use a genetic mouse model of tamoxifen-inducible CreERT2 knocked into the terminally differentiated SC-specific promoter Sox10 and the TdTomato reporter gene Sox10-iCre^{ERT2/+};R26-LSL-TdT. This “Sox10-TdT” strain allows visualization of corneal nerve-ensheathing SCs when tamoxifen is applied to the cornea in vivo. Following tamoxifen administration, the left cornea will be denervated and, starting from day 0, topically treated with 100 ng/ml NRG1 or vehicle-only. Note: all in vivo experiments will be performed on the left cornea, while the right eye will serve as an internal positive control. Five days after denervation, the epithelium will be removed with an Amoils brush³. Corneal epithelial recovery will be assessed daily by fluorescein staining for four days. To prevent accidental self-wounding by scratching the anesthetic cornea, a tarsorrhaphy (eyelid suture) will be maintained. In the inventors' preliminary study, and consistent with reported results, corneal denervation caused SC withdrawal in 48 hours. The inventors' initial in vivo fluorescent microscopy observations demonstrate that NRG1 induced survival of SCs in limbus at levels comparable to those of healthy non-denervated corneas (FIG. 10B-D). In turn, this NRG1-mediated survival of SCs induced healing of deepithelialized denervated corneas within 36 hours, comparable to the healing observed in normally innervated corneas in rats (FIG. 10E,F) and mice (not shown). Consistent with the inventors' previous observations, vehicle-only treated corneas exhibited delayed wound healing and developed corneal opacifications (FIG. 10 5E,F). As a control, the inventors used Thy1-YFP transgenic mice, which express YFP in axons, to ensure that NRG1 treatment did not inhibit Wallerian degeneration following corneal denervation (FIG. 10A). These data correlate with immunostaining observations, demonstrating complete axonal loss in the Sox10-TdT denervated NRG1-treated mouse corneas (FIG. 10C). Note, to assess changes in SC volume, the inventors measured total TdT-positive limbal areas in fixed experimental corneas, including fluorescent cell debris in denervated vehicle-only treated conditions. This method probably overestimated SC survival in the control vehicle-only treatment conditions (FIG. 10D). To improve accuracy, the inventors will repeat this experiment on more biological samples, while complementing the analysis of cell survival using TUNEL staining. Although further quantitative and qualitative analysis is required to validate these preliminary results, this gain-of-function experiment supports the inventors' loss-of-function results³, correlating the presence of SCs and corneal epithelial healing capacity. Further quantitative analysis will be performed to correlate SC numbers with corneal healing capacity. Caveat: NRG1 treatment might not be effective in more prolonged post-denervation period(s). It might be associated with reduction in the number of survived SCs and/or their gradual de-differentiation to SC precursors (that change their secretome), as occurs in wounded skin or digit tip, thereby failing to provide a sufficient trophic support. This possibility will be addressed in Aim 3.

Sub-Aim 1b: To define the role of SCs in LSC activity using both loss- and gain-of-function approaches. 1b.1. Working Hypothesis: SCs directly regulate LSC maintenance and activity. 1b.1.1. Approach: The inventors will induce either local genetic SC ablation or surgical corneal denervation, with or without topical NRG1, to determine the extent to which SCs regulate the number and activity of LSC independent of corneal sensory neurons. 1b.1.2. Experimental Design and Expected Results: Corneal denervation causes a 75% reduction in cells expressing LSC markers such as ABCG2, p63 and Krt15. The extent to which LSC maintenance and activity depend on corneal nerve-associated SCs, as well as the mechanism of this relationship, is unknown. Therefore, in a loss-of-function experiment, the inventors will use transgenic Sox10-DTA;K15-EGFP mice derived from crossing Sox10-DTA mice with keratin 15 (Krt or K15)-EGFP mice (which constitutively express enhanced green fluorescent protein (EGFP) under the LSC-specific complex 1 acidic gene 15 promoter). An image of a cornea containing K15-EGFP-expressing LSCs is presented in FIG. 14. EGFP-positive LSCs in the Sox10-DTA;K15-EGFP mouse enable us to count LSCs after SC ablation. The inventors will ablate SCs by weekly application of topical tamoxifen for one month. During this time, the inventors will track in vivo the location and morphology of EGFP-positive LSCs in intact and de-epithelialized corneas. Sox10-DTA;K15-EGFP non-tamoxifen treated mice will serve as positive controls. Next, as in the gain-of-function experiments proposed in Aim 1a.1, the inventors will denervate corneas in K15-EGFP mice, then track the location and morphology of EGFP-positive LSCs, while comparing between NRG1 and vehicle-only treated corneas. Following the in vivo fluorescent microscopic assessments, the inventors will immunostain the corneas for LSC-specific markers, including ABCG2, p63 and Krt15, as well as the cell proliferation marker Ki67 in harvested corneas at days 10, 20 and 30 post-tamoxifen administration and the inventors will localize Ki67/EGFP cells that are triple-positive. Also, bIII tubulin immunostaining will be performed to visualize the corneal axons that are not affected by SC ablation (FIG. 13). The inventors anticipate that, as after corneal denervation, SC ablation will reduce LSC proliferation and or differentiation, and that this effect will be more pronounced in de-epithelialized corneas. Correlating with preserving epithelial recovery capacity, the inventors anticipate that NRG1 will prolong LSC survival in denervated corneas, due to trophic preservation of SCs. Caveat: Similarly to Aim 1a, in prolonged denervation NRG1-treated corneas, the LSC pool will probably progressively reduce, due to reduction in the number of surviving SCs. The inventors will address this possibility in Aim 3.

Sub-Aim 1c: To define SCs as an integral regulatory component of the LSC niche. 1c.1. Working Hypothesis: SCs regulate the activity of the LSC niche. 1c.1.1. Approach: The inventors will induce local genetic SC ablation or surgical corneal denervation, applied alone or together with topical trophic treatment, to observe the role of corneal innervation, particularly SCs, in the activity of the LSC niche. 1c.1.2. Preliminary Observations and Experimental Design: Previously the inventors used a lineage tracing approach to quantitatively trace epithelial renewal. This was achieved by crossing R26R-Confetti mice with Krt14-Cre^{ERT/+} mice (K14-Confetti) that allowed us to induce, with topical tamoxifen, an irreversible roadblock removal and excision or inversion of a fluorescent dimer, thereby allowing the random expression of one out of four reporter genes in each K14 positive basal epithelial cells, including LSCs. The inventors crossed this mouse with the K15-EGFP mouse to generate the triple transgenic K14-Confetti;K15-EGFP. The inventors used this mouse to demonstrate that physical removal of EGFP-positive LSCs caused terminally-differentiated K14-positive cells to back-migrate into the limbal niche and de-differentiate into EGFP-expressing LSCs, restoring the stem cell pool (FIG. 14). The inventors' scRNA-seq-based observations also suggested possible juxtaparacrine interactions between SCs and MSCs and, therefore, that SCs are involved in regulation of the LSC niche activity (FIG. 9). To determine whether de-differentiation of epithelial cells into LSCs depends on corneal innervation and, if so, whether SCs are involved in this process, the inventors will repeat this LSC pool recovery experiment, comparing (1) normally-innervated K15-EGFP corneas, (2) denervated K15-EGFP corneas, (3) denervated K15-EGFP corneas topically treated with NRG1, and (4) normally-innervated SC-ablated Sox10-DTA;K15-EGFP corneas in mice. Seven days after corneal denervation or ten days after first tamoxifen administration in experimental groups (2, 3) and (4), respectively, the inventors will remove a ring-shaped EGFP-positive LSC-bearing zone, the marginal conjunctiva, and the marginal cornea under a fluorescent microscope using an ophthalmic rotating burr (Algerbrush), as the inventors previously described. As before, the inventors will then track re-establishment of the EGFP-positive pool of LSCs in vivo¹. The inventors anticipate that, following physical removal of LSCs, either corneal denervation or SC ablation will interfere with re-establishing the LSC pool, which will recover in normally innervated or denervated NRG1 treated corneas.

AIM 2: To validate pleiotrophin as a novel regulator of LSCs. Multiple cytokines and growth factors are upregulated during corneal epithelial renewal that may mediate corneal epithelial maintenance and wound healing via regulating either epithelial cell migration or LSC proliferation/self-renewal. Based on the factor library yielded by the inventors' recent scRNA-seq computational screening predicting paracrine SC-LSC interactions (FIG. 9), the inventors chose to narrow the inventors' focus to three known factors: platelet-derived growth factor a (PDGF-a), transforming growth factor-β (TGF-b) and ciliary neurotrophic factor (CNTF), plus the novel candidate factor, pleiotrophin (PTN), which has not been previously implicated in corneal epithelial renewal. By applying these factors, the inventors will measure, in gain-of-function experiments, their effects on (a) epithelial healing in denervated or SC-ablated deepithelialized corneas, and (b) maintenance and activity of LSCs in vivo in mice and (c) primary hLSC in vitro. Clinical relevance: As a novel therapeutic target, combinations of factors may supplement or supplant the role of SCs in treating human NK.

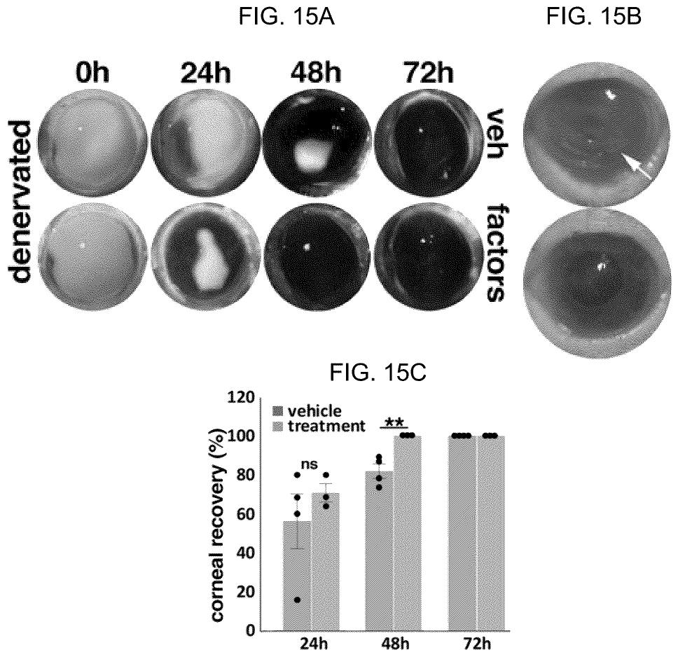

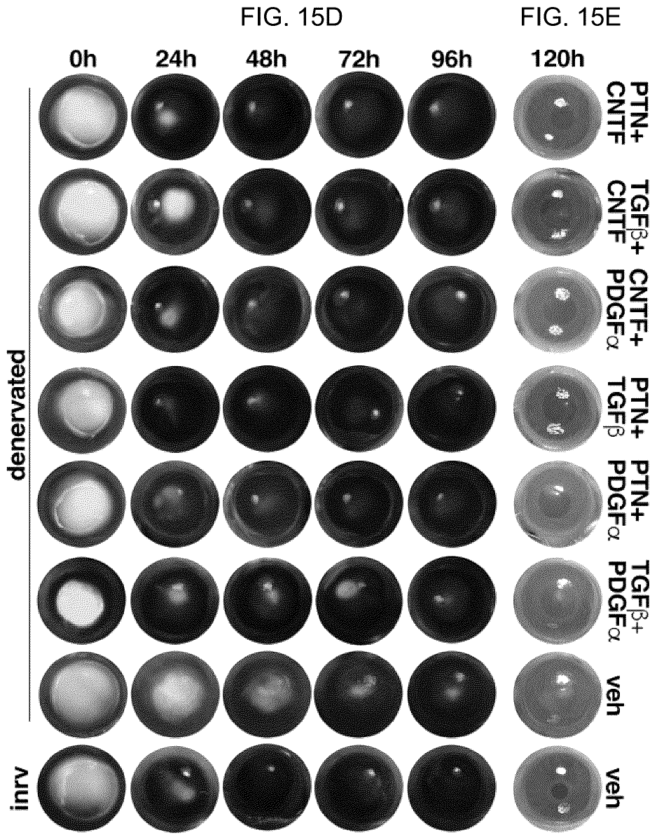

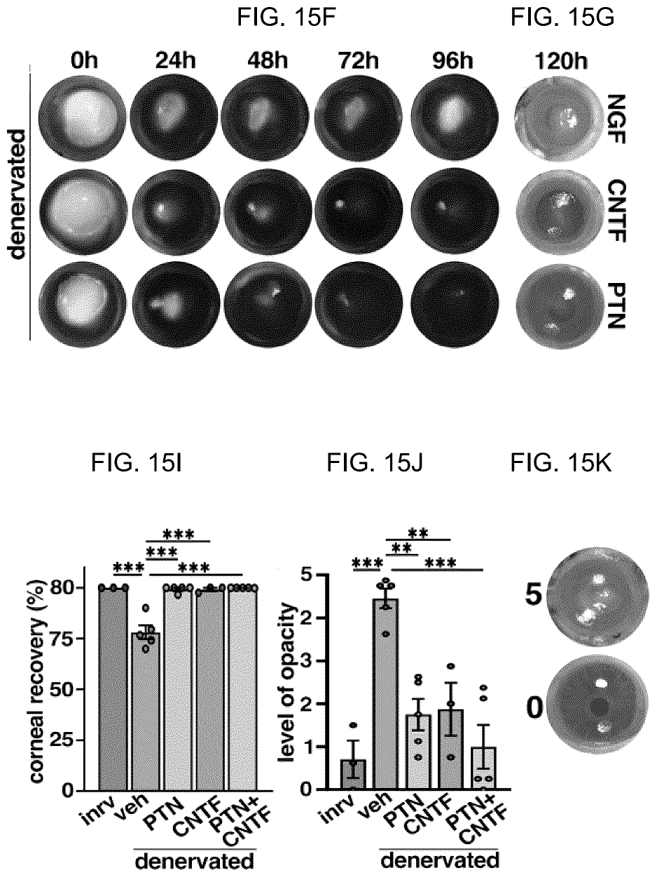

Sub-Aim 2a: To define the effect of selected SC-derived trophic factors on corneal epithelial renewal. 2a.1. Working Hypothesis: Trophic factors expressed by SCs mediate corneal innervation-dependent epithelial recovery. 2a.1.1. Approach: The inventors will determine the effect of treatment with either single factors or different combinations of the factors on recovery of experimentally denervated deepithelialized rat corneas. 2a.1.2. Preliminary Observations and Experimental Plan: The inventors performed a series of preliminary screening experiments, using the factor library yielded by the previous scRNA-Seq studies (FIG. 15). The inventors tested the effects of daily doses of the selected factors on epithelial recovery five days post-denervation in rat corneas and identified those that improved wound healing. In combinatorial in vivo assays, corneas were treated immediately after denervation and every 24 hours with 5 ml of (a) a mixture of all four factors, containing PTN (0.2 ng/ml), CNTF (10 ng/ml), TGF-b1 (0.4 ng/ml), and PDGF-a (40 ng/ml), (b) the six possible combinations of two combined factors, and (c) single factors applied alone. Treatment with one daily dose of NGF (50 mg/ml) was used as a control. On day five after denervation, the corneal epithelium was removed with a brush. Corneal wound healing was assessed daily with fluorescein staining. The inventors' preliminary results demonstrate that combined topical administration of the four factors together caused complete epithelial wound healing in 48 hours. The treated corneas remained completely transparent and evenly shaped (FIG. 15A-C), indistinguishable from the healing of normally innervated control corneas (FIG. 15D,E). The vehicle control group's corneas became opaque (probably due to epithelial keratinization, like the human NK phenotype) (FIG. 15B). The inventors' preliminary results indicate that every combination of two factors that contained CNTF or PTN induced complete corneal recovery in 48 hours (FIG. 15D,I). Likewise, each of these two factors alone induced complete healing of denervated deepithelialized cornea within 48 hours (FIG. 15F,I). However, this healing yielded traces of scar tissue by 120 hours after deepithelialization (FIG. 15E,G,J). Notably, similar to the four combined factors, only the combination of PTN with CNTF induced complete recovery of clear epithelium (FIG. 15D,E,I,J). Interestingly, in denervated rat corneas treated with PDGF-a and TGF-b1 (FIG. 15D), or PDGF-a alone (not shown), a marked wound healing response occurred in the first 24 hours. However, like in human NK, healing progressed slowly and resulted in corneal opacification (FIG. 15E). Surprisingly, treatment with daily NGF had no effect on epithelial recovery, indistinguishable from vehicle-treated controls (FIG. 15F,G). Similarly to the treatment with NRG1 (FIG. 10A), neither of combinations of the topically applied factors supported spontaneous re-innervation of the denervated corneas, as indicated by immunostaining for bIII tubulin of harvested fixed corneas (FIG. 10H). Overall, although further qualitative and quantitative analysis is required, these preliminary results demonstrate rapid wound healing in completely denervated corneas using topical trophic factors. Moreover, these observations corroborate the scRNA-seq-based findings, suggesting that PTN is a novel regulator of innervation-dependent corneal epithelial renewal. Note: although the inventors used one daily dose (instead of six daily doses of NGF as used clinically), the failure of NGF to heal denervated corneas (i) suggests that PTN and CNTF alone or combined are more potent in supporting corneal healing, (ii) may explain cenegermin's failure to treat 30% of NK cases and (iii) suggests that NGF mostly acts on improving corneal innervation in partially anesthetic NK corneas.

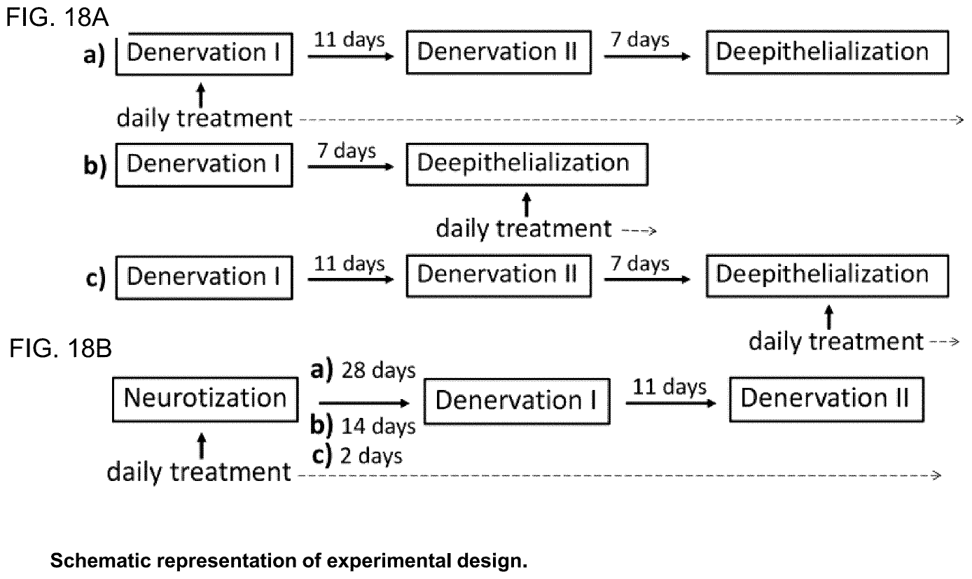

Notably in the inventors' NK model, the inventors observed both corneal ulceration and also stromal opacification (not shown), analogous to the stromal melting observed in NK patients. This observation suggests that treatment with the factors (in FIG. 10D-K) also prevents stromal degradation. To address possible changes to the stroma, the inventors will supplement the above experimental conditions' epithelial healing analysis by morphological and immunohistochemical analysis of the stroma. 2a.2. Working Hypothesis: SCs trophically support corneal epithelial renewal. 2a.2.1. Approach: To directly examine the correlation between corneal denervation-generated trophic deficits (FIG. 10) and absence of SCs, and thereby, implicate SCs as the trophic regulators of corneal epithelial renewal, in a gain-of-function experiment, the inventors will examine healing of genetically SC-ablated mouse corneas, treated with PTN and CNTF alone or together. 2a.2.2. Experimental Plan and Initial Results: Like in Aim 1b, the inventors will induce apoptosis of corneal SCs that also express TdT in Sox10-DTA;TdT transgenic mice (FIGS. 10A and 18A). Starting from the second day after the first tamoxifen administration and during the duration of the experiment, the inventors will treat the corneas with combinations of four or two trophic factors as in Aim 2a.1.2. Ten days after the first tamoxifen administration, the inventors will de-epithelialize the SC-ablated corneas. As reported, the induced expression of Diphtheria toxin significantly reduces the number of corneal SCs (FIG. 16A), thereby interfering with corneal wound healing. The inventors' initial results (n=2) indicate that, similarly to denervated corneas, treatment with the four factors induced complete wound healing in 48 hours (FIG. 16B). Likewise, and comparably to SC-intact vehicle-only treated wt deepithelialized corneas, those treated with PTN and CNTF healed in 24 hours (FIG. 16C). As a negative control, the inventors use SC-ablated corneas treated with NRG1. These corneas are not expected to heal, due to absence of SCs. Accordingly, and unlike in experimentally denervated wt corneas (FIG. 10E,F), NRG1 failed to rescue the healing of SC-ablated corneas, which remained wounded after 96 hours and developed opaque scar tissue (FIG. 16C). Although more biological replicates are required for statistical validation, these initial observations provide evidence supporting the key role of SCs in regulating corneal epithelial renewal. These observations also support the hypothesis suggesting that SCs trophically regulate the activity of LSCs. To define the individual contribution of each factor, SC-ablated corneas will be deepithelialized and treated with single factors.

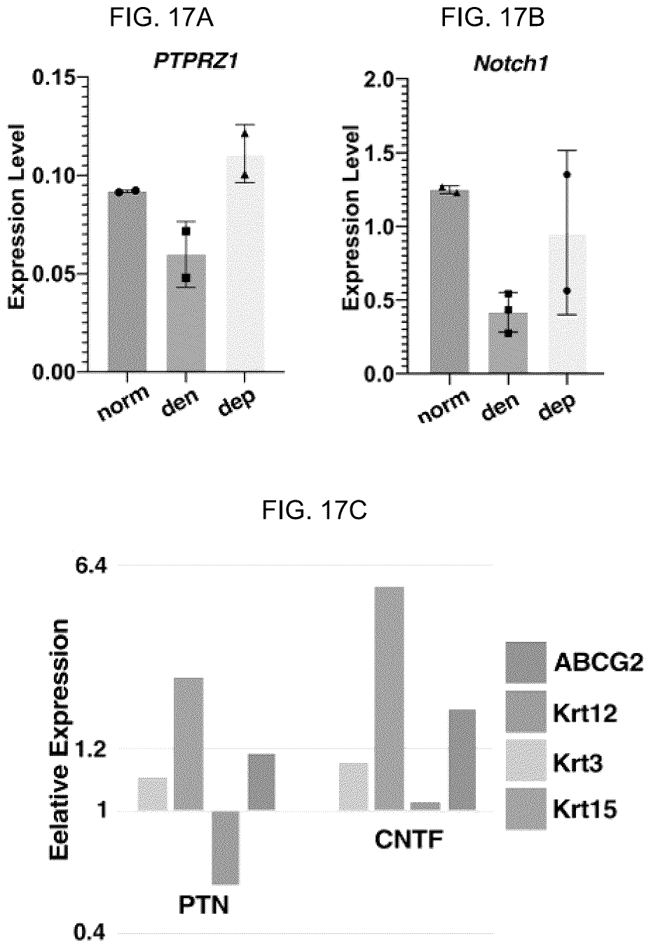

Sub-Aim 2b: To identify the role of PTN in LSC activity. How might these trophic factors affect LSC?CNTF, which induces LSCs self-renewal and the epithelial cell migration, and PTN, which induces epithelial cell differentiation and stem cell self-renewal in other tissues might work synergistically to regulate LSC activity. In the inventors' scRNA-seq analysis, the inventors observed a 100-fold increase in PTN expression in SCs following corneal de-epithelialization, unlike CNTF, whose expression remained stable in all experimental conditions. These initial results indicate different mechanisms of activity for these two factors during homeostatic or injury-induced epithelial renewal. The inventors' initial analysis of the scRNA-seq data also indicate expression of the most common PTN receptors, protein tyrosine phosphatase zeta 1 (PTPRZ1) (FIG. 17A) and syndecan-3 (SND3) (not shown) by the cluster of LSCs, which are involved in multiple developmental and homeostatic processes, including proliferation, migration and progenitor cell self-renewal. Moreover, syndican-1 (SND1) that also interacts interact with PTN, was directly shown to be involved in regulation of corneal epithelial cells proliferation and migration, critical for the epithelial wound healing. 2b.1. Working Hypothesis: PTN expressed by SCs acts synergistically with CNTF to regulate LSC maintenance and activity. 2b.1.1. Approach: To validate expression of PTN by SCs and validate upregulation of its transcription following de-epithelialization, as well as the expression of PTN receptors by LSCs, in 2b.1.2. the inventors will perform in situ hybridization, real time quantitative PCR (RT-qPCR) and immunostaining of whole-mount corneas with a focus on the limbal area. To define the role of PTN in regulation of LSC activity, in 2b.1.3. the inventors will treat genetically LSC-labeled denervated mouse corneas with PTN or CNTF alone or combined, while measuring LSC proliferation and differentiation, and migration of LSC progeny. To correlate rodents-based in vivo results with those of human cells and to demonstrate a direct effect of PTN on LSCs, in 2b.1.4., cultured hLSC will be treated with PTN and CNTF either alone or together, and the effect of the treatment on hLSC expression pattern and clonogenicity will be assessed. Finally, to confirm PTN signaling in LSCs in a loss-of-function experiment, the inventors will examine the effect of knock-down of PTPRZ1, SND1 and SND3 expression on survival and clonogenicity of hLSC in presence or absence of PTN. 2b.1.2. Experimental Plan and Expected Results: Previously, the inventors performed fluorescent in situ hybridization (FISH) and immunohistochemistry to label Sox10 mRNA- and protein-expressing SCs, respectively, in whole-mount corneas. To observe PTN expression in corneal SCs and its receptors by LSCs, the inventors will repeat the fluorescent labeling analyses, double labeling for the two cells' specific markers Sox10, S100b, together with PTN or Krt15, ABCG2, together with PTPRZ1 or SND1 or SND3, respectively. To detect changes in PTN expression in transcriptional and translational levels, RT-qPCR and Western blot analyses will be performed on limbi, isolated from healthy, deepithelialized, and denervated (negative control) mouse corneas, as the inventors did in the scRNA-seq studies³. The inventors anticipate that the expression of PTN and PTPRZ1 will be observed at both the transcriptional and translational levels in SCs and in LSCs, respectively, and that corneal deepithelialization will cause upregulation of PTN expression. Note, as demonstrated by scRNA-seq analysis, expression of PTN was also detected in MSCs, so the inventors are open to the possibility that FISH visualization of PTN might not be restricted to SCs alone. However, since no upregulation in the transcriptional expression of PTN was detected following de-epithelialization and, since, based on the inventors' initial results, exogenous PTN induced epithelial healing in denervated and SC ablated rodent corneas (FIGS. 17 and 18), SCs may be the exclusive source of PTN protein. 2b.1.3. Experimental Plan and Expected Results: As the inventors reported and described in Aim 1c.1.2., the inventors will use K14-Confetti;K15-EGFP triple transgenic mice (FIG. 14) to measure the extent to which PTN and CNTF, applied alone or together, are involved in innervation-regulated LSC proliferation and differentiation. In normally innervated deepithelialized K14-Confetti corneas, every randomly colored LSC gives rise to epithelial cell progeny that gradually differentiate and migrate towards the corneal center in the form of an unequally colored single radial stripe. The inventors will denervate, and, five days later, de-epithelialize the corneas while treating them with either PTN only, CNTF only, PTN+CNTF, or vehicle alone during the entire duration of the experiment. One day before and on the day of denervation, the corneas will be treated with tamoxifen to induce Cre activation. Normally innervated deepithelialized tamoxifen-treated K14-Confetti;K15-EGFP corneas will serve as controls. Following de-epithelialization, the experimental corneas will be observed microscopically in vivo for the appearance of new EGFP-positive LSCs, as well as new progeny migrating towards the corneal center as manifested by fluorescent cell colonies in the form of stripes. The extent of LSC clones' survival will be defined by measuring (i) the size of every single clone and (ii) the number of differently colored clones. The rate of corneal epithelial renewal will be defined by the pace of appearance of radial monochrome stripes (FIG. 14). On the 5^{th} day after de-epithelialization, the corneas will be harvested and co-immunolabeled for LSC markers (ABCG2, Krt-15, p63) and Ki67 to define LSC proliferation. Based on previous reports and the presented denervated cornea healing preliminary results (FIG. 15), the inventors anticipate CNTF and PTN will complement each other in inducing LSC self-renewal and differentiation. To more specifically identify the role of every of the two factors on LSCs activity, the inventors will expand the above analysis on isolated LSCs, as proposed in the next section.

Notch signaling induces differentiation of LSCs. The inventors' scRNA-seq analysis indicates a significant downregulation in Notch expression by LSCs following denervation (FIG. 17). The inventors will validate the changes in limbal Notch expression following corneal denervation by RT-qPCR performed on mRNA extracted from the limbal are of the experimental corneas. The inventors will then use RT-qPCR approach to examine changes in Notch expression in denervated or genetically-induced SC ablated untreated, or different combinations of the factor-treated corneas. The inventors anticipate that limbal Notch expression will drop following corneal denervation or SC-ablation and will be rescued by either or every of the trophic treatments. 2b.1.4. Experimental Plan, Initial and Expected Results: The in vivo experiment in 2b.1.3. will be complemented by examining the effect of the trophic factor(s) on cultured hLSCs. LSCs characteristically form long-term proliferative large clones, known as holoclones. Previously, the inventors used hLSC cultures to validate the inventors' mouse study derived results addressing stemness and differentiation of LSCs. The inventors will harvest hLSCs as described. The inventors will then assess the hLSCs' potential to survive and form colonies, comparing between treatment with PTN and CNTF. Media lacking either of the factors will be used as a negative control. The inventors will perform RT-qPCR to examine the effect of the experimental conditions on expression of LSC maintenance and differentiation markers, including Notch by hLSCs. Finally, as reported, the inventors will apply endoribonuclease-prepared silencing RNA (esiRNA) approach, asking how knock-down of either of the three mentioned above PTN receptors expression will affect the identity and clonogenicity of hLSCs, comparing between PTN-present or absent conditions. The inventors' initial RT-qPCR results demonstrate that both CNTF and PTN induce transcriptional upregulation in two LSC-specific markers Krt15 and ABCG2, as well as corneal epithelial cells-specific marker Krt3. At the same time, PTN induced a slight downregulation of another corneal epithelial cells-specific marker Krt12. While yet to be statistically validated and supported by expression and immunohistochemical analysis, these initial results (i) support the inventors' in vivo observations, suggesting PTN as a novel regulator of LSC activity and (ii) indicate that PTN acts synergistically with CNTF to both support survival and controlled differentiation of LSCs. The inventors anticipate that treatment with both factors together will induce upregulation in Notch expression. Based on these initial results together with corneal healing mediated by the treatment with PTN alone or together with CNTF, the inventors anticipate that treatment with either of factors, and even more when combined, will support survival and clonogenicity of the cultured hLSCs. Caveat. Previous report so and the inventors' preliminary data (FIG. 9), suggest involvement of multiple trophic factors in regulation of LSCs. Therefore, the inventors cannot exclude the possibility that PTN and CNTF's actions in preventing NK and supporting LSCs is, although critical, not the only existing pathway to support corneal epithelial renewal. Since it is beyond the focus of this research proposal, this possibility will be addressed in the inventors' further research.

AIM 3. Develop a comprehensive therapeutic approach to treat NK. The inventors' preliminary and initial results demonstrate that, either preservation of nerve-associated SCs with topical NRG1 (FIG. 10) or, instead, compensation for SC withdrawal with topical PTN and CNTF (FIG. 15), prevent development of NK within ten days after experimental corneal denervation. However, to be relevant to most clinical cases, usually presenting with longstanding NK, chronic conditions must be considered. For example, NRG1 might not be sufficient to maintain limbal SCs for the more prolonged post-denervation periods. Or, alternatively, neuron-dissociated de-differentiated SCs might change their secretome during prolonged dissociation from the axons, and, thereby, fail to prevent an NK phenotype at later time points. Additionally, although topical pharmacological treatment might be effective in NK patients with partial corneal denervation, surgical corneal neurotization may, as is currently often the case, still be indicated for patients with completely denervated corneas. These patients suffer from complete lack of corneal innervation and, due to recurrent corneal injuries and opacification, combined with major intrinsic deficits in wound healing ability, inevitably progress to vision loss. However, in these patients, corneal neurotization may become even more challenging due to (i) limited axonal growth capacity, as seen in many congenital cases and/or (ii) the time delay between the neurotization procedure and the arrival of innervation into the denervated cornea, due to the long distance that the donor nerve axons need to pass through the nerve graft. In these situations, a prolonged deficit in corneal nerve-derived trophic support causes substantial loss of LSCs, leading to inevitable damage to the corneal epithelium, necessitating corneal transplantation after neurotization. An interesting observation is provided by those cases in which neurotization is followed by corneal transplantation when the original cornea has become opaque. In these cases, the host keeps the original limbus, while the donor cornea does not include the limbus. This observation suggests that in cases of successful neurotization, donor corneal clarity is maintained in tandem with reactivation of the limbal niche activity. This conclusion is complemented by the inventors' recent discovery that following physical removal of LSCs, a functional limbal niche is capable of restoring the LSC pool by inducing de-differentiation of corneal-committed cells back to functional LSCs (also see the experimental plan of Sub-Aim 1c).

Sub-Aim 3a. To develop a therapeutic approach to prevent chronic NK. 3a.1. Working Hypothesis. Topical treatment with the selected trophic factors will prevent development of NK in chronically denervated cornea. 3a.1.1. Approach. In the inventors' NK rat model, there is a spontaneous corneal re-innervation occurs in about three weeks following a single electrocautery of the ophthalmomaxillary branch of the trigeminal nerve. To model chronic NK in human patients, the inventors will induce prolonged corneal denervation by two constitutive electrocauteries in intervals of 11 days in rats, as the inventors described (further addresses as “chronic” denervation). Similarly as in Aim 2, the inventors will treat chronically denervated deepithelialized cornea with PTN, CNTF or both factors together either during the whole course of the experiment or one week after the first denervation, once the denervated cornea have lost its regenerative capacity. 3a.1.2. Experimental Design and Expected Results. One week after the second nerve electrocautery, the inventors will induce corneal de-epithelialization and track healing. As in 2a.1.2., corneas will (a) be treated with either of the factor combinations during the whole course of the experiment, starting with the first (of two constitutive) nerve electrocautery (FIG. 18A). This experiment will define whether NK can be prevented in chronically anesthetic corneas by exogenously provided factors that, otherwise, absent in denervated corneas. To define the treatment of existing NK in either freshly or chronically denervated anesthetic corneas, the inventors will repeat the experiment as above, but instead, will start the treatment on the day of deepithelialization either (b) seven days after single or (c) one week after the second nerve electrocautery, i.e. in chronically denervated corneas (FIG. 18A). The experimental groups will be compared to those of rats with denervated or normally innervated vehicle-only treated deepithelialized corneas. The inventors anticipate that combining factors will rescue the NK phenotype more effectively in chronic NK.

Sub-Aim 3b. To improve the efficacy of corneal neurotization-based treatment of NK by topical treatment. One of the corneal neurotization's limitations in humans, is the time it takes the donor axons to reach the cornea. The inventors have observed that the longer corneas are denervated, the worse the outcomes following neurotization, possibly in part due to prolonged lack of LSC maintenance in the absence of SC. 3b.1. Working Hypothesis. The inventors hypothesize that topical treatment with trophic factors will improve the efficacy of corneal neurotization by maintaining the functionality of the limbal niche until corneal re-innervation occurs. 3b.1.1. Approach. To better model clinical NK conditions, in the proposed study the inventors will increase the time elapsed between denervation and re-innervation of a neurotized cornea. The inventors will perform corneal neurotization in two groups of animals with a different time delay between corneal denervation and neurotization-induced re-innervation, comparing between untreated and topical trophic factor-treated corneas. 3b.1.2 Experimental Plan and Expected Results. To model the clinical situation, when corneal neurotization is performed in either chronically denervated corneas or shortly after corneal denervation (e.g., in cases of recent brain tumor resection), the inventors will perform corneal neurotization procedures in two experimental groups of rats: (b) two weeks or (c) two days prior to corneal denervation (FIG. 18B). This will allow a gap of nearly one or three weeks, respectively, for the donor nerves to innervate the limbal area and nearly two or four weeks to innervate the central cornea. This time gap will be sufficient for LSC loss or/and development of an NK phenotype. Starting from the day of corneal denervation, the corneas will be topically treated daily with (i) PTN+CNTF; to support LSC survival and activity directly, (ii) NRG1; that, via supporting SCs will both induce LSC survival and activity, as well as support re-innervation of the necrotized cornea and (iii) PTN+CNTF+NRG1; to both support SC survival and provide sufficient trophic support to LSCs that might be required after the longer time gap between denervation and neurotization. To prevent corneal physical damage, a tarsorrhaphy will be maintained during the duration of the experiment. Three weeks after neurotization, the experimental corneas will be deepithelialized, and their healing will be assessed during the ensuing four days. Then the corneas will be harvested and immunostained for bIII tubulin (axons) and LSC markers to determine the extent of corneal neurotization and numbers of LSCs, respectively. The inventors anticipate that (i) treatment with PTN+CNTF will induce corneal healing and maintain clarity in proportion to numbers of LSCs in both experimental groups, and (ii) treatment with NRG1 will induce corneal healing and LSC survival in group a, but may fail to do so in group b, following SC gradual withdrawal and/or dedifferentiation⁷. Treatment with all three factors together will be especially beneficial in group b, supporting both LSC survival and activity, as well as SC-mediated support of corneal re-innervation. If treatment with the factors indeed induces epithelial recovery in corneas exposed to three weeks of complete denervation, the inventors will repeat this experiment in corneas exposed to a time gap of 1.5 months between denervation and neurotization. Caveat. Although angiogenic activity of PTN is controversial, it may induce angiogenesis in pathological conditions. In the inventors' experimental system, by topically applying PTN to experimentally denervated deepithelialized corneas, the inventors are re-establishing a natural trophic milieu. In fact, no neovascularization was observed during the inventors' initial study, following 10 days of daily treatment with PTN. Moreover, in the inventors' system, treatment with PTN prevented neovascularization that usually occurs during NK in human and rodents (not shown). However, despite the listed indications arguing against PTN angiogenetic activity in cornea, this option needs to be addressed and excluded when developing topical treatment to NK. Therefore, in this Aim, the inventors will assess for neovascularization the corneas of the experimental NK animals treated with PTN, using morphological observation and immunohistochemistry approaches.

SUMMARY: The mechanistic role of SCs and their interaction with LSC in NK is not well understood. A research program will therefore be undertaken in which the mechanistic crosstalk between SCs and LSCs will be defined. Building on recent breakthroughs that allowed us to screen and identify a new library of intercellular communication pathways, as well as new surgical and genetic models of NK, the inventors will integrate chemical, biochemical, biophysical and genetic tools to obtain deep understanding into the molecular mechanisms underlying NK. The program will move forward in three directions, namely 1) using gain- and loss-of-function experiments to alter SC numbers and activity to determine their impact on corneal wound healing, 2) determining the impact of topical application of known and novel (in cornea, e.g., PTN) predicted SC gene products on LSC activity, and 3) validating novel therapeutics as new treatments for early intervention for NK patients with either new onset or chronic NK. These studies will define the mechanisms of corneal epithelial maintenance and will inform development of novel rational therapeutics for patients with NK.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### REFERENCES

1 Hanna, C., Bicknell, D. S. & O'Brien, J. E. Cell turnover in the adult human eye. Arch Ophthalmol 65, 695-698 (1961).
2 Cenedella, R. J. & Fleschner, C. R. Kinetics of corneal epithelium turnover in vivo. Studies of lovastatin. Invest Ophthalmol Vis Sci 31, 1957-1962 (1990).
3 Bentley, A. J. et al. Characterization of human corneal stem cells by synchrotron infrared micro-spectroscopy. Mol Vis 13, 237-242 (2007).
4 Chen, Z. et al. Characterization of putative stem cell phenotype in human limbal epithelia. Stem Cells 22, 355-366, doi:10.1634/stemcells.22-3-355 (2004).
5 Schlotzer-Schrehardt, U. & Kruse, F. E. Identification and characterization of limbal stem cells. Exp Eye Res 81, 247-264, doi:10.1016/j.exer.2005.02.016 (2005).
6 Romano, A. C. et al. Different cell sizes in human limbal and central corneal basal epithelia measured by confocal microscopy and flow cytometry. Invest Ophthalmol Vis Sci 44, 5125-5129 (2003).
7 Catapano, J., Antonyshyn, K., Zhang, J. J., Gordon, T. & Borschel, G. H. Corneal Neurotization Improves Ocular Surface Health in a Novel Rat Model of Neurotrophic Keratopathy and Corneal Neurotization. Invest Ophthalmol Vis Sci 59, 4345-4354, doi:10.1167/iovs.18-24843 (2018).
8 Pellegrini, G. et al. Location and clonal analysis of stem cells and their differentiated progeny in the human ocular surface. J Cell Biol 145, 769-782 (1999).
9 Yazdanpanah, G. et al. Strategies for reconstructing the limbal stem cell niche. Ocul Surf 17, 230-240, doi:10.1016/j.jtos.2019.01.002 (2019).
10 Mann, I. A Study of Epithelial Regeneration in the Living Eye. Br J Ophthalmol 28, 26-40 (1944).
11 Davanger, M. & Evensen, A. Role of the pericorneal papillary structure in renewal of corneal epithelium. Nature 229, 560-561 (1971).
12 Pfister, R. R. & Burstein, N. The alkali burned cornea I. Epithelial and stromal repair. Exp Eye Res 23, 519-535 (1976).
13 Buck, R. C. Cell migration in repair of mouse corneal epithelium. Invest Ophthalmol Vis Sci 18, 767-784 (1979).
14 Buck, R. C. Measurement of centripetal migration of normal corneal epithelial cells in the mouse. Invest Ophthalmol Vis Sci 26, 1296-1299 (1985).
15 Collinson, J. M., Chanas, S. A., Hill, R. E. & West, J. D. Corneal development, limbal stem cell function, and corneal epithelial cell migration in the Pax6(+/−) mouse. Invest Ophthalmol Vis Sci 45, 1101-1108 (2004).
16 Nagasaki, T. & Zhao, J. Centripetal movement of corneal epithelial cells in the normal adult mouse. Invest Ophthalmol Vis Sci 44, 558-566 (2003).
17 Di Girolamo, N. et al. Tracing the fate of limbal epithelial progenitor cells in the murine cornea. Stem Cells 33, 157-169, doi:10.1002/stem.1769 (2015).
18 Cotsarelis, G., Cheng, S. Z., Dong, G., Sun, T. T. & Lavker, R. M. Existence of slow-cycling limbal epithelial basal cells that can be preferentially stimulated to proliferate: implications on epithelial stem cells. Cell 57, 201-209 (1989).
19 Zhao, J., Mo, V. & Nagasaki, T. Distribution of label-retaining cells in the limbal epithelium of a mouse eye. J Histochem Cytochem 57, 177-185, doi:10.1369/jhc.2008.952390 (2009).
20 Pajoohesh-Ganji, A., Pal-Ghosh, S., Simmens, S. J. & Stepp, M. A. Integrins in slow-cycling corneal epithelial cells at the limbus in the mouse. Stem Cells 24, 1075-1086, doi:10.1634/stemcells.2005-0382 (2006).
21 Figueira, E. C., Di Girolamo, N., Coroneo, M. T. & Wakefield, D. The phenotype of limbal epithelial stem cells. Invest Ophthalmol Vis Sci 48, 144-156, doi:10.1167/iovs.06-0346 (2007).
22 Raji, B. et al. The RNA-binding protein Musashi-1 is produced in the developing and adult mouse eye. Mol Vis 13, 1412-1427 (2007).
23 Thomas, P. B. et al. Identification of Notch-1 expression in the limbal basal epithelium. Mol Vis 13, 337-344 (2007).
24 Di Girolamo, N. et al. Localization of the low-affinity nerve growth factor receptor p75 in human limbal epithelial cells. J Cell Mol Med 12, 2799-2811, doi:10.1111/j.1582-4934.2008.00290.x (2008).
Barbaro, V. et al. C/EBPdelta regulates cell cycle and self-renewal of human limbal stem cells. J Cell Biol 177, 1037-1049, doi:10.1083/jcb.200703003 (2007).
26 Di Iorio, E. et al. Isoforms of DeltaNp63 and the migration of ocular limbal cells in human corneal regeneration. Proc Natl Acad Sci USA 102, 9523-9528, doi:10.1073/pnas.0503437102 (2005).
27 Peterson, D. C. & Hamel, R. N. in StatPearls (2022).
28 Muller, L. J., Marfurt, C. F., Kruse, F. & Tervo, T. M. Corneal nerves: structure, contents and function. Exp Eye Res 76, 521-542 (2003).
29 Cavanagh, H. D. & Colley, A. M. The molecular basis of neurotrophic keratitis. Acta Ophthalmol Suppl 192, 115-134 (1989).
Bonini, S., Rama, P., Olzi, D. & Lambiase, A. Neurotrophic keratitis. Eye (Lond) 17, 989-995, doi:10.1038/sj.eye.6700616 (2003).
31 Sigelman, S. & Friedenwald, J. S. Mitotic and wound-healing activities of the corneal epithelium; effect of sensory denervation. AMA Arch Ophthalmol 52, 46-57 (1954).
32 Ueno, H. et al. Dependence of corneal stem/progenitor cells on ocular surface innervation. Invest Ophthalmol Vis Sci 53, 867-872, doi:10.1167/iovs.11-8438 (2012).
33 Alper, M. G. The anesthetic eye: an investigation of changes in the anterior ocular segment of the monkey caused by interrupting the trigeminal nerve at various levels along its course. Trans Am Ophthalmol Soc 73, 323-365 (1975).
34 Beuerman, R. W. & Schimmelpfennig, B. Sensory denervation of the rabbit cornea affects epithelial properties. Exp Neurol 69, 196-201 (1980).
35 Sacchetti, M. & Lambiase, A. Diagnosis and management of neurotrophic keratitis. Clin Ophthalmol 8, 571-579, doi:10.2147/OPTH.S45921 (2014).
36 Dana, R. et al. Expert consensus on the identification, diagnosis, and treatment of neurotrophic keratopathy. BMC Ophthalmol 21, 327, doi:10.1186/si2886-021-02092-1 (2021).
37 Catapano, J. et al. Treatment of neurotrophic keratopathy with minimally invasive corneal neurotisation: long-term clinical outcomes and evidence of corneal reinnervation. Br J Ophthalmol, doi:10.1136/bjophthalmol-2018-313042 (2019).
38 Adachi, W. et al. Serial analysis of gene expression (SAGE) in the rat limbal and central corneal epithelium. Invest Ophthalmol Vis Sci 47, 3801-3810, doi:10.1167/iovs.06-0216 (2006).
39 Altshuler, A. et al. Discrete limbal epithelial stem cell populations mediate corneal homeostasis and wound healing. Cell Stem Cell 28, 1248-1261 e1248, doi:10.1016/j.stem.2021.04.003 (2021).
40 Amitai-Lange, A. et al. Lineage tracing of stem and progenitor cells of the murine corneal epithelium. Stem Cells 33, 230-239, doi: 10.1002/stem.1840 (2015).
41 Nasser, W. et al. Corneal-Committed Cells Restore the Stem Cell Pool and Tissue Boundary following Injury. Cell Rep 22, 323-331, doi:10.1016/j.celrep.2017.12.040 (2018).
42 Farrelly, O. et al. Two-photon live imaging of single corneal stem cells reveals compartmentalized organization of the limbal niche. Cell Stem Cell 28, 1233-1247 e1234, doi:10.1016/j.stem.2021.02.022 (2021).
43 Li, W., Hayashida, Y., Chen, Y. T. & Tseng, S. C. Niche regulation of corneal epithelial stem cells at the limbus. Cell Res 17, 26-36, doi:10.1038/sj.cr.7310137 (2007).
44 Johnston, A. P. et al. Sox2-mediated regulation of adult neural crest precursors and skin repair. Stem Cell Reports 1, 38-45, doi:10.1016/j.stemcr.2013.04.004 (2013).
45 Johnston, A. P. et al. Dedifferentiated Schwann Cell Precursors Secreting Paracrine Factors Are Required for Regeneration of the Mammalian Digit Tip. Cell Stem Cell 19, 433-448, doi:10.1016/j.stem.2016.06.002 (2016).
46 Nakamura, M. et al. Synergistic effect of substance P with epidermal growth factor on epithelial migration in rabbit cornea. Exp Eye Res 65, 321-329, doi:10.1006/exer.1997.0345 (1997).
47 Yamada, N., Yanai, R., Inui, M. & Nishida, T. Sensitizing effect of substance P on corneal epithelial migration induced by IGF-1, fibronectin, or interleukin-6. Invest Ophthalmol Vis Sci 46, 833-839, doi:10.1167/iovs.04-0775 (2005).
48 Murphy, C. J. et al. Spontaneous chronic corneal epithelial defects (SCCED) in dogs: clinical features, innervation, and effect of topical SP, with or without IGF-1. Invest Ophthalmol Vis Sci 42, 2252-2261 (2001).
49 Marfurt, C. F. & Echtenkamp, S. F. The effect of diabetes on neuropeptide content in the rat cornea and iris. Invest Ophthalmol Vis Sci 36, 1100-1106 (1995).
50 Elbadri, A. A., Shaw, C., Johnston, C. F., Archer, D. B. & Buchanan, K. D. The distribution of neuropeptides in the ocular tissues of several mammals: a comparative study. Comp Biochem Physiol C 100, 625-627 (1991).
51 Yang, L. et al. Substance P promotes diabetic corneal epithelial wound healing through molecular mechanisms mediated via the neurokinin-1 receptor. Diabetes 63, 4262-4274, doi:10.2337/dbl4-0163 (2014).
52 Marfurt, C. F., Kingsley, R. E. & Echtenkamp, S. E. Sensory and sympathetic innervation of the mammalian cornea. A retrograde tracing study. Invest Ophthalmol Vis Sci 30, 461-472 (1989).
53 Jones, M. A. & Marfurt, C. F. Calcitonin gene-related peptide and corneal innervation: a developmental study in the rat. J Comp Neurol 313, 132-150, doi:10.1002/cne.903130110 (1991).
54 Garcia-Hirschfeld, J., Lopez-Briones, L. G. & Belmonte, C. Neurotrophic influences on corneal epithelial cells. Exp Eye Res 59, 597-605, doi:10.1006/exer.1994.1145 (1994).
55 Lambiase, A., Rama, P., Bonini, S., Caprioglio, G. & Aloe, L. Topical treatment with nerve growth factor for corneal neurotrophic ulcers. N Engl J Med 338, 1174-1180, doi:10.1056/NEJM199804233381702 (1998).
56 Lambiase, A. et al. Nerve growth factor promotes corneal healing: structural, biochemical, and molecular analyses of rat and human corneas. Invest Ophthalmol Vis Sci 41, 1063-1069 (2000).
57 Tan, M. H., Bryars, J. & Moore, J. Use of nerve growth factor to treat congenital neurotrophic corneal ulceration. Cornea 25, 352-355, doi:10.1097/01.ico.0000176609.42838.df (2006).
58 Ivanusic, J. J., Wood, R. J. & Brock, J. A. Sensory and sympathetic innervation of the mouse and guinea pig corneal epithelium. J Comp Neurol 521, 877-893, doi:10.1002/cne.23207 (2013).
59 Pflugfelder, S. C. et al. Topical Recombinant Human Nerve Growth Factor (Cenegermin) for Neurotrophic Keratopathy: A Multicenter Randomized Vehicle-Controlled Pivotal Trial. Ophthalmology 127, 14-26, doi:10.1016/j.ophtha.2019.08.020 (2020).
60 Kaplan, D. R. & Miller, F. D. Neurotrophin signal transduction in the nervous system. Curr Opin Neurobiol 10, 381-391 (2000).
61 Touhami, A., Grueterich, M. & Tseng, S. C. The role of NGF signaling in human limbal epithelium expanded by amniotic membrane culture. Invest Ophthalmol Vis Sci 43, 987-994 (2002).
62 Bandtlow, C. E., Heumann, R., Schwab, M. E. & Thoenen, H. Cellular localization of nerve growth factor synthesis by in situ hybridization. EMBO J 6, 891-899 (1987).
63 Elbaz, U., Bains, R., Zuker, R. M., Borschel, G. H. & Ali, A. Restoration of corneal sensation with regional nerve transfers and nerve grafts: a new approach to a difficult problem. JAMA Ophthalmol 132, 1289-1295, doi:10.1001/jamaophthalmol.2014.2316 (2014).
64 Bains, R. D., Elbaz, U., Zuker, R. M., Ali, A. & Borschel, G. H. Corneal neurotization from the supratrochlear nerve with sural nerve grafts: a minimally invasive approach. Plast Reconstr Surg 135, 397e-400e, doi:10.1097/PRS.0000000000000994 (2015).
65 Yuzwa, S. A. et al. Developmental Emergence of Adult Neural Stem Cells as Revealed by Single-Cell Transcriptional Profiling. Cell Rep 21, 3970-3986, doi:10.1016/j.celrep.2017.12.017 (2017).
66 Chen, S. Y. et al. Pax 6 Controls Neural Crest Potential of Limbal Niche Cells to Support Self-Renewal of Limbal Epithelial Stem Cells. Sci Rep 9, 9763, doi:10.1038/s41598-019-45100-7 (2019).
67 Guo, P. et al. Limbal niche cells are a potent resource of adult mesenchymal progenitors. J Cell Mol Med 22, 3315-3322, doi:10.1111/jcmm.13635 (2018).
68 Li, D. Q. et al. Single-cell transcriptomics identifies limbal stem cell population and cell types mapping its differentiation trajectory in limbal basal epithelium of human cornea. Ocul Surf 20, 20-32, doi:10.1016/j.jtos.2020.12.004 (2021).
69 Bargagna-Mohan, P. et al. Corneal nonmyelinating Schwann cells illuminated by single-cell transcriptomics and visualized by protein biomarkers. J Neurosci Res 99, 731-749, doi:10.1002/jnr.24757 (2021).
70 Yamazaki, S. et al. Nonmyelinating Schwann cells maintain hematopoietic stem cell hibernation in the bone marrow niche. Cell 147, 1146-1158, doi:10.1016/j.cell.2011.09.053 (2011).
71 Boyd, J. G. & Gordon, T. Glial cell line-derived neurotrophic factor and brain-derived neurotrophic factor sustain the axonal regeneration of chronically axotomized motoneurons in vivo. Exp Neurol 183, 610-619 (2003).
72 Boyd, J. G. & Gordon, T. Neurotrophic factors and their receptors in axonal regeneration and functional recovery after peripheral nerve injury. Mol Neurobiol 27, 277-324, doi:10.1385/MN:27:3:277 (2003).
73 Ferrari, G. et al. A novel mouse model for neurotrophic keratopathy: trigeminal nerve stereotactic electrolysis through the brain. Invest Ophthalmol Vis Sci 52, 2532-2539, doi:10.1167/iovs.10-5688 (2011).
74 Morris, R. J. et al. Capturing and profiling adult hair follicle stem cells. Nat Biotechnol 22, 411-417, doi:10.1038/nbt950 (2004).
75 Feinberg, K. et al. A neuroprotective agent that inactivates prodegenerative TrkA and preserves mitochondria. J Cell Biol 216, 3655-3675, doi:10.1083/jcb.201705085 (2017).
76 Sheha, H., Tighe, S., Hashem, O. & Hayashida, Y. Update On Cenegermin Eye Drops In The Treatment Of Neurotrophic Keratitis. Clin Ophthalmol 13, 1973-1980, doi:10.2147/OPTH.S185184 (2019).
77 Cattin, A. L. & Lloyd, A. C. The multicellular complexity of peripheral nerve regeneration. Curr Opin Neurobiol 39, 38-46, doi:10.1016/j.conb.2016.04.005 (2016).
78 Carr, M. J. et al. Mesenchymal Precursor Cells in Adult Nerves Contribute to Mammalian Tissue Repair and Regeneration. Cell Stem Cell 24, 240-256 e249, doi:10.1016/j.stem.2018.10.024 (2019).
79 Yuzwa, S. A. et al. Proneurogenic Ligands Defined by Modeling Developing Cortex Growth Factor Communication Networks. Neuron 91, 988-1004, doi:10.1016/j.neuron.2016.07.037 (2016).
80 Ali, N. et al. Regulatory T Cells in Skin Facilitate Epithelial Stem Cell Differentiation. Cell 169, 1119-1129.e1111, doi:10.1016/j.cell.2017.05.002 (2017).
81 Tang, C. et al. Neural Stem Cells Behave as a Functional Niche for the Maturation of Newborn Neurons through the Secretion of PTN. Neuron 101, 32-44.e36, doi:10.1016/j.neuron.2018.10.051 (2019).
82 Bertram, S. et al. Pleiotrophin increases neurite length and number of spiral ganglion neurons in vitro. Exp Brain Res 237, 2983-2993, doi:10.1007/s00221-019-05644-6 (2019).
83 Haber, M. et al. Effects of growth factors (EGF, PDGF-BB and TGF-beta 1) on cultured equine epithelial cells and keratocytes: implications for wound healing. Vet Ophthalmol 6, 211-217, doi:10.1046/j.1463-5224.2003.00296.x (2003).
84 Zhou, Q. et al. Ciliary neurotrophic factor promotes the activation of corneal epithelial stem/progenitor cells and accelerates corneal epithelial wound healing. Stem Cells 33, 1566-1576, doi: 10. 1002/stem. 1942 (2015).
85 Pellegrii, G. et al. p63 identifies keratinocyte stem cells. Proc Natl Acad Sci USA 98, 3156-3161, doi:10.1073/pnas.061032098 (2001).
86 Barrandon, Y. & Green, H. Three clonal types of keratinocyte with different capacities for multiplication. Proc Natl Acad Sci USA 84, 2302-2306, doi: 10. 1073/pnas. 84.8.2302 (1987).
87 Bhattacharya, S. et al. SOX2 Regulates P63 and Stem/Progenitor Cell State in the Corneal Epithelium. Stem Cells 37, 417-429, doi: 10. 1002/stem.2959 (2019).

SEQUENCES

| SEQ ID | | |
|---|---|---|
| NO | Description | Sequence |
| 1 | Human CNTF | MAFTEHSPLTPHRRDLCSRSIWLARKIRSDLTALTESYVK |
| | | HQGLNKNINLDSADGMPVASTDQWSELTEAERLQENLQ |
| | | AYRTFHVLLARLLEDQQVHFTPTEGDFHQAIHTLLLQVA |
| | | AFAYQIEELMILLEYKIPRNEADGMPINVGDGGLFEKKL |
| | | WGLKVLQELSQWTVRSIHDLRFISSHQTGIPARGSHYIAN |
| | | NKKM |
| 2 | Human PTN | MQAQQYQQQRRKFAAAFLAFIFILAAVDTAEAGKKEKP |
| | | EKKVKKSDCGEWQWSVCVPTSGDCGLGTREGTRTGAE |
| | | CKQTMKTQRCKIPCNWKKQFGAECKYQFQAWGECDLN |
| | | TALKTRTGSLKRALHNAECQKTVTISKPCGKLTKPKPQA |
| | | ESKKKKKEGKKQEKMLD |
| 3 | Human | MPPSGLRLLLLLLPLLWLLVLTPGRPAAGLSTCKTIDMEL |
| | TGFB1 | VKRKRIEAIRGQILSKLRLASPPSQGEVPPGPLPEAVLALY |
| | | NSTRDRVAGESAEPEPEPEADYYAKEVTRVLMVETHNEI |
| | | YDKFKQSTHSIYMFFNTSELREAVPEPVLLSRAELRLLRL |
| | | KLKVEQHVELYQKYSNNSWRYLSNRLLAPSDSPEWLSF |
| | | DVTGVVRQWLSRGGEIEGFRLSAHCSCDSRDNTLQVDIN |
| | | GFTTGRRGDLATIHGMNRPFLLLMATPLERAQHLQSSRH |
| | | RRALDTNYCFSSTEKNCCVRQLYIDFRKDLGWKWIHEP |
| | | KGYHANFCLGPCPYIWSLDTQYSKVLALYNQHNPGASA |
| | | APCCVPQALEPLPIVYYVGRKPKVEQLSNMIVRSCKCS |
| 4 | Human | MRTLACLLLLGCGYLAHVLAEEAEIPREVIERLARSQIHS |
| | PDGFA | IRDLQRLLEIDSVGSEDSLDTSLRAHGVHATKHVPEKRPL |
| | | PIRRKRSIEEAVPAVCKTRTVIYEIPRSQVDPTSANFLIWP |
| | | PCVEVKRCTGCCNTSSVKCQPSRVHHRSVKVAKVEYVR |
| | | KKPKLKEVQVRLEEHLECACATTSLNPDYREEDTGRPRE |
| | | SGKKRKRKRLKPT |

We claim:

1. A pharmaceutical composition comprising ciliary neurotrophic factor (CNTF) and at least one additional trophic factor selected from the group consisting of: pleiotrophin (PTN), transforming growth factor beta 1 (TGFb1), and platelet-derived growth factor alpha (PDGFa).

2. A pharmaceutical composition comprising pleiotrophin (PTN) formulated for topical ophthalmic administration.

3. The pharmaceutical composition of claim 1, wherein the at least one trophic factor comprises PTN.

4. The pharmaceutical composition of claim 3, wherein the at least one trophic factor consists of PTN.

5. (canceled)

6. The pharmaceutical composition of claim 1, wherein the pharmaceutical composition comprises:
pleiotrophin (PTN);
ciliary neurotrophic factor (CNTF);
transforming growth factor beta 1 (TGFb1); and
platelet-derived growth factor alpha (PDGFa).

7. (canceled)

8. The pharmaceutical composition of claim 1, wherein the composition is formulated for topical ophthalmic administration.

9. (canceled)

10. A method of treating neurotrophic keratopathy in a subject in need thereof, the method comprising administering a therapeutically effective amount of the pharmaceutical composition of claim 1 to the subject to treat the neurotrophic keratopathy.

11. The method of claim 10, wherein the pharmaceutical composition comprises PTN.

12. A method of improving corneal healing in a subject in need thereof, the method comprising administering a therapeutically effective amount of the pharmaceutical composition of claim 1 to the subject to improve corneal healing in the subject.

13. A method of improving corneal transparency after corneal injury in a subject in need thereof, the method comprising administering a therapeutically effective amount of the pharmaceutical composition of claim 1 to the subject to restore corneal transparency after corneal injury in the subject.

14. A method of improving corneal epithelial renewal in a subject in need thereof, the method comprising administering a therapeutically effective amount of the pharmaceutical composition of claim 1 to the subject to improve corneal epithelial renewal in the subject.

15. The method of claim 10, wherein the subject is suffering from diabetes.

16. A method of treating diabetic keratopathy in a subject in need thereof, the method comprising administering a therapeutically effective amount of the pharmaceutical composition of claim 1 to the subject to treat the diabetic keratopathy.

17. A method of treating corneal complications of diabetes in a subject in need thereof, the method comprising administering a therapeutically effective amount of the pharmaceutical composition of claim 1 to the subject to treat corneal complications of diabetes in the subject.

18. A method of delaying the onset of diabetic keratopathy in a subject in need thereof, the method comprising administering a therapeutically effective amount of the pharmaceutical composition of claim 1 to the subject to delay the onset of diabetic keratopathy in the subject.

19. The method of claim 10, wherein administration comprises topical ophthalmic administration.

20.-23. (canceled)

24. The method of claim 10, wherein the method improves corneal transparency in the subject.

25. The method of claim 10, wherein the subject has about 50% or less corneal innervation, about 40% or less corneal innervation, about 30% or less corneal innervation, about 20% or less corneal innervation, about 10% or less corneal innervation, or wherein the subject has no corneal innervation.

26. The method of claim 10, wherein the subject suffers from corneal anesthesia.

27.-28. (canceled)

29. The method of claim 10, wherein the method results in complete recovery of the corneal epithelium.


SEQUENCE LISTING (4 sequences)

SEQ ID NO: 1 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 2 - protein, 168 aa, Homo sapiens
MQAQQYQQQRRKFAAAFLAFIFILAAVDTAEAGKKEKPEKKVKKSDCGEWQWSVCVPTSGDCGLGTREGTRTGAECKQTMKTQRCKIPCNWKKQFGAECKYQFQAWGECDLNTALKTRTGSLKRALHNAECQKTVTISKPCGKLTKPKPQAESKKKKKEGKKQEKMLD

SEQ ID NO: 3 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 4 - protein, 211 aa, Homo sapiens [sequence omitted]